(19) United States
(12) Patent Application Publication
(10) Pub. No.: US 2011/0053166 A1
(43) Pub. Date: Mar. 3, 2011

(54) STEM CELL EXPRESSION CASSETTES

(75) Inventors: James Ellis, Toronto (CA); Akitsu Hotta, Kyoto (JP)

(73) Assignee: The Hospital for Sick Children, Toronto, ON (CA)

(21) Appl. No.: 12/920,059

(22) PCT Filed: Feb. 27, 2009
(86) PCT No.: PCT/CA2009/000230

§ 371 Date: Oct. 22, 2010

Related U.S. Application Data
(60) Provisional application No. 61/064,366, filed on Feb. 29, 2008.

(30) Foreign Application Priority Data
Mar. 6, 2008 (CA) 2,621,155

(51) Int. Cl. C12Q 1/68; C07H 21/00; C12N 15/63; C12N 5/10; C12N 15/85; C12N 15/06; C12Q 1/04

(52) U.S. Cl. 435/6; 536/23.5; 435/320.1; 435/325; 435/455; 435/449; 435/34

(57) ABSTRACT

A stem cell expression cassette, comprising a nucleic acid comprising a pluripotent stem cell specific promoter, and a tag sequence, wherein the pluripotent stem cell specific promoter and tag sequences are operatively linked, is provided. Also provided are methods of identifying and methods of selecting a pluripotent cell, using the stem cell expression cassette.

### FIELD OF INVENTION

The present invention relates to nucleic acid sequences comprising regulatory sequences that direct expression of tag sequences in stem cells. The invention furthermore provides nucleic acid sequences that direct expression in embryonic or induced pluripotent stem cells.

### BACKGROUND OF THE INVENTION

Embryonic stem cell-like pluripotent stem cells, called induced Pluripotent Stem (iPS) cells, can be induced by introducing 1 to 4 genes into somatic cells using retroviral vectors in vitro (see, for example Okita et al., 2007). This ‘reprogramming’ of iPS cells is inefficient, and optimization may be desirable. The unique morphology of mouse and human iPS cells can be used to isolate reprogrammed cells, however distinguishing of iPS morphology requires substantial experience of stem cell culture to allow for ease of identification (Takahashi et al 2007). More consistent methods of identification of iPS cells are needed.

In order to facilitate identification of reprogrammed cells, they may be screened for the expression of markers expressed at the embryonic stage, for example SSEA-1 (stage-specific embryonic antigen-1) for mouse iPS cells; SSEA-3 (stage-specific embryonic antigen-3), SSEA-4 (stage-specific embryonic antigen-4), TAR-1-60 or TAR-1-81 for human iPS. Those surface markers may not directly reflect the reprogrammed nuclear state, since there is no functional link between surface markers and pluripotency. Use of an antibody to interact with the surface marker may adversely affect the cell, and, depending on the details of the method used, the tested cells may not be viable. Since the efficiency of reprogramming is very low, a method to enrich a population of cells for reprogrammed cells when sorting may be useful.

Viral vectors, such as retroviral vectors, represent efficient vehicles for introduction of foreign nucleic acid into iPS cells. Retroviral transgene expression after integration, however, tends to be silenced or attenuated in pluripotent stem cells, such as embryonic stem cells (ES), embryonic carcinoma cells (EC) and iPS cells (see, for example, Yao et al., 2004; Okita, supra; Wernig et al., 2007; Meissner et al., 2007), thus conveying a marker that is intended to be expressed only in ES or iPS cells in such a vehicle may be counterproductive.

Stem cells are cells that retain the ability to self-renew (undergo multiple cycles of cell division while maintaining an undifferentiated state), and are capable of differentiation into other cell lineages or specialized cell types (potency). Embryonic stem cells (ES cells, or ES) are stem cells found at the blastocyst stage of embryonic development. ES cells generally have the potential to differentiate into any or all of the specialized embryonic tissues in any of the three primary germ layers—endoderm, ectoderm, and mesoderm.

A pluripotent stem cell is capable of giving rise to any or all of the various cell types that make up the body, but cannot normally differentiate into extraembryonic tissues.

Both human ES cells (hES cells, or hES) and mouse or murine ES cells (mES cells, or mES) are the subject of research—both have key stem cell characteristics of pluripotency and self-renewal. The growth conditions and markers required for each differ however—for example, mES may be grown on a layer of gelatin, and require the presence of LIF (leukemia inhibitory factor) in the culture medium, while hES generally require a feeder layer of mouse embryonic fibroblasts (MEF), and FGF-2 (fibroblast growth factor-2) in the culture medium. Thus, experimental manipulations that are demonstrated to work in mES do not always transfer to a human system—the outcome may be unpredictable.

Human ES or mES, when injected directly into a subject, will differentiate into a variety of cell types, and form a generally disorganized mass referred to as a teratoma. In order for hES or mES to be used in therapeutic applications, or even as a consistent source of experimental material, differentiation must be controlled to provide for useable cells. Residual undifferentiated cells must be killed or otherwise removed to prevent teratoma formation after transplantation into a subject.

A variety of protocols for differentiating ES into specific cell types are known, and the selection of a suitable protocol may depend on the source of the ES (e.g. human or mouse, or other species), the desired tissue, cell type or developmental stage that the ES is to be differentiated into, or the desired end use of the differentiated cell. See for example, Current Protocols in Stem Cell Biology (Wiley Interscience)

An iPS is a pluripotent stem cell artificially derived from an adult somatic cell, through introduction of specific transcription factors. Methods of inducing pluripotent stem cells from mouse and human fibroblasts are described in, for example Takahashi, supra; and Takahashi and Yamanaka, 2006, both of which are herein incorporated by reference. These methods involve introduction of pluripotency factors into human or murine fibroblasts. Pluripotency factors include transcription factors that, when expressed in a somatic cell, result in the reprogramming of the cell and induce it to develop into a pluripotent state.

A vehicle for introducing nucleic acid sequences to be expressed specifically in ES or iPS cells is desired.

### SUMMARY OF THE INVENTION

The present invention relates to nucleic acid sequences comprising regulatory sequences that direct expression of tag sequences in stem cells. The invention furthermore provides nucleic acid sequences that direct expression in embryonic or induced pluripotent stem cells.

In accordance with one aspect of the invention, there is provided a nucleic acid comprising a pluripotent stem cell specific promoter, operatively linked to a tag sequence. The pluripotent stem cell-specific promoter may be an ETn promoter sequence (SEQ ID NO: 1), an ETn poly A mutated (pAMu) promoter sequence (SEQ ID NO: 2), or other pluripotent stem-cell specific promoter.

In accordance with another aspect of the invention, the nucleic acid may further comprise one or more than one pluripotent stem cell specific enhancer sequence. A pluripotent stem cell specific enhancer sequence is an enhancer sequence active in a pluripotent stem cell. Each of the pluripotent stem cell specific enhancer sequences is operatively linked to the pluripotent stem cell specific promoter and may be in a forward (positive or “+”) or reverse (negative or “−”) orientation. The one or more than one pluripotent stem cell specific enhancer sequence may be operatively linked 5′ or 3′ relative to the promoter, or the tag sequence, or both the promoter and tag sequence. The pluripotent stem cell specific enhancer sequence may be CR4, SRR2, a combination of CR4 and SRR2, or may be selected from the group comprising SEQ ID NO: 3, SEQ ID NO: 4, SEQ ID NO: 5, and SEQ ID NO: 6.

In accordance with another aspect of the invention, there is provided a nucleic acid comprising an ETn poly A mutated (pAMu) promoter sequence (SEQ ID NO: 2) operatively linked to a tag sequence and a first sequence, the first sequence comprising an enhancer motif active in a pluripotent stem cell. The enhancer motif may be selected from the group consisting of two or more than two SRR2 enhancer sequences, and one or more than one CR4 enhancer sequence. The enhancer motif may be located upstream of the pAMu promoter sequence.

In accordance with another aspect of the invention, the one or more than one CR4 enhancer sequence is selected from the group consisting of SEQ ID NOS: 3 and 5.

In accordance with another aspect of the invention, the two or more than two SRR2 enhancer sequence is selected from the group consisting of SEQ ID NOS: 4 and 6.

In accordance with another aspect of the invention, the tag sequence may encode an amino acid sequence permitting antibiotic selection, drug selection, color selection, negative selection, cell-surface selection or fluorescence selection. The tag sequence may alternately encode a pluripotency factor, or a differentiation factor to drive differentiation into specific cell lineages (for example MyoD directs differentiation into muscle).

In accordance with another aspect of the invention, there is provided a cell comprising a nucleic acid, the nucleic acid comprising a pluripotent stem cell specific promoter, operatively linked to a tag sequence. The pluripotent stem cell-specific promoter may be an ETn promoter sequence (SEQ ID NO: 1), an ETn poly A mutated (pAMu) promoter sequence (SEQ ID NO: 2), or other pluripotent stem-cell specific promoter.

In accordance with another aspect of the invention, the cell may be an adult somatic cell, such as, but not limited to a fibroblast, a keratinocyte, a cynoviocyte, a mesenchymal stem cell, a neural stem/progenitor cell, a skin progenitor cell, a hepatocyte, a gastric epithelial cell, a pluripotent stem cell, or an induced pluripotent stem cell.

In accordance with another aspect of the invention, there is provided a vector comprising a nucleic acid, the nucleic acid comprising a pluripotent stem cell specific promoter, operatively linked to a tag sequence. The pluripotent stem cell-specific promoter may be an ETn promoter sequence (SEQ ID NO: 1), an ETn poly A mutated (pAMu) promoter sequence (SEQ ID NO: 2), or other pluripotent stem-cell specific promoter.

In accordance with another aspect of the invention, the vector may be a viral vector, such as a retroviral or lentiviral vector. The vector may further be a self-inactivating vector.

In accordance with another aspect of the invention, there is provided a stem cell expression vector comprising, an ETn pAMu promoter sequence operatively linked to a tag sequence, and one or more enhancer sequences, the one or more than one enhancer sequence selected from the group comprising CR4, SRR2, and a combination of CR4 and SRR2.

In accordance with another aspect of the invention, there is provided a method of producing an induced pluripotent stem cell comprising, inducing pluripotency to a cell with one or more than one pluripotency factor to produce a pluripotent cell, transfecting the pluripotent cell with a nucleic acid comprising a pluripotent stem cell specific promoter operatively linked to a tag sequence, to produce a transfected cell, growing the transfected cell, and selecting for the induced pluripotent stem cell.

A cell may be induced to become pluripotent by transfection with one or more than one pluripotency factor, or by transfection with one or more than one vector encoding the one or more than one pluripotency factor. A cell may be induced to become pluripotent by exposure to one or more than one pluripotency factor in a culture medium.

In accordance with another aspect of the invention, there is provided a method of producing an induced pluripotent stem cell comprising, transfecting a cell with a nucleic acid, the nucleic acid comprising a pluripotent stem cell specific promoter operatively linked to a tag sequence to produce a transfected cell, inducing pluripotency to the transfected cell with one or more than one pluripotency factor to produce the induced pluripotent stem cell, and growing the induced pluripotent stem cell.

In accordance with another aspect of the invention, there is provided a method of producing an induced pluripotent stem cell comprising either: Ai) reprogramming a cell to induce pluripotency producing a pluripotent cell; Aii) transfecting the pluripotent cell with the nucleic acid of claim 1 to produce a transfected pluripotent cell; or Bi) transfecting a cell with the nucleic acid of claim 1 to produce a transfected cell; Bii) reprogramming a cell to induce pluripotency to produce a transfected pluripotent cell; iii) growing the transfected pluripotent cell; and iv) selecting for an induced pluripotent stem cell.

According to some embodiments of the invention, the step of reprogramming may comprise: transfecting a cell with one or more than one pluripotency factors; adding one or more than one chemical, cytokine, or hormone into culture medium of a cell; transfecting a cell with one or more than one pluripotency factors and adding one or more than one chemical, cytokine, or hormone into culture medium of a cell; nuclear transfer of a cell into a pluripotent stem cell or an oocyte; or cell-cell fusion of a cell with a pluripotent stem cell.

In accordance with another aspect of the invention, there is provided a method of identifying a pluripotent stem cell comprising, providing a population of pluripotent stem cells, transfecting the population of pluripotent stem cells with a nucleic acid comprising a pluripotent stem cell specific promoter, operatively linked to a tag sequence, expressing the nucleic acid and selecting for an amino acid sequence of interest encoded by a tag sequence.

In accordance with another aspect of the invention, there is provided a method of overcoming silencing of one or more than one gene or nucleotide sequence following retroviral transfection, the method comprising, transfecting an adult fibroblast or an embryonic stem cell with a vector comprising a nucleic acid, the nucleic acid comprising a pluripotent stem cell specific promoter operatively linked to a tag sequence, and expressing the nucleic acid thereby overcoming silencing of the one or more gene or nucleotide sequence.

In accordance with another aspect of the invention, there is provided a stem cell expression cassette comprising, a nucleic acid, the nucleic acid comprising a pluripotent stem cell specific promoter, operatively linked to a tag sequence.

In accordance with another aspect of the invention, there is provided a method of maintaining a pluripotent stem cell in a pluripotent state comprising, providing a population of pluripotent stem cells comprising a nucleic acid, the nucleic acid comprising a pluripotent stem cell specific promoter operatively linked to a tag sequence, and expressing the nucleic acid thereby maintaining the pluripotent stem cell in the pluripotent state.

In accordance with another aspect of the invention, there is provided a method of purging one or more than one undifferentiated pluripotent stem cell from a population of differentiated stem cells during directed differentiation comprising, providing a population of pluripotent stem cells each comprising a nucleic acid, the nucleic acid comprising a pluripotent stem cell specific promoter operatively linked to a tag sequence, expressing the nucleic acid and differentiating the population of pluripotent stem cells, and killing any pluripotent stem cells that continue to express an amino acid sequence of interest encoded by the tag sequence of the nucleic acid thereby purging the one or more than one undifferentiated pluripotent stem cell from the population of differentiated stem cells.

In accordance with another aspect of the invention, there is provided a method for identifying a potential pluripotency factor comprising, providing a population of cells comprising a nucleic acid, the nucleic acid comprising a pluripotent stem cell specific promoter operatively linked to a tag sequence, exposing the population of cells to media comprising the potential pluripotency factor, expressing the nucleic acid and selecting for a pluripotent cell expressing an amino acid sequence of interest encoded by the tag sequence of the nucleic acid, whereby selection of the pluripotent cell expressing the tag sequence is indicative of the occurrence of the potential pluripotency factor in the media.

In accordance with another aspect of the invention, there is provided a kit for identification, production, or both identification and production, of a pluripotent stem cell or an embryonic stem cell, the kit comprising a nucleic acid comprising an ETn pAMu promoter sequence operatively linked to a tag sequence and instructions for its use. The kit may further comprise one or more than one pluripotency factor, media, other agents useful in selecting a pluripotent stem cell, or a combination thereof. The kit may further provide one or more than one nucleic acid comprising a sequence encoding one or more than one pluripotency factor. The kit may further comprise one or more than one transfection reagent for transfecting a cell.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows that a retroviral vector with ETn (Early Transposon) promoter has higher viral titer and expression level than that with Nanog or Oct-4 promoter in ES cells. FIG. 1A, shows a schematic illustration of HSC-1 retroviral vectors. The nucleic acid sequence of the start codon of EGFP is altered into a Kozak consensus sequence. Oct-4, Sox2, Sp1 binding sites and hormone responsive element (HRE) are indicated. Ψ (psi): packaging signal, Δ (delta)gag: extended packaging signal, CR1-3: conserved region 1-3 of Oct-4 promoter. FIG. 1B, shows that a retroviral vector with ETn pAMu promoter has the highest titer in mouse ES (Embryonic Stem) cells among tested ES-specific promoters. Each retroviral vector was infected into J1 mouse ES cells and NIH3T3 fibroblasts simultaneously, and 2 or 3 days after infection, the percentage of EGFP (Enhanced Green Fluorescent Protein) positive cells were analyzed by flow cytometry. Viral titers were calculated as described in the method section, and normalized by the titer of HSC1-PGK-EGFP control virus in NIH3T3 cells. Graphs are an average from three independent viral preps and error bars indicate standard deviation. FIG. 1C, shows that a ETn pAMu promoter has the highest EGFP expression level in mouse ES cells among tested ES-specific promoters. Each retroviral vector was infected into J1 ES cells and NIH3T3 fibroblasts simultaneously, and 2 or 3 days after infection, mean fluorescence intensity (MFI) of GFP positive population was analyzed by flow cytometry. Graphs are an average from three independent viral stocks and error bars indicate standard deviation.

FIG. 2 shows that Nanog and Oct-4 promoters suppress expression of viral vectors in virus producer cells. FIG. 2A, shows EGFP expression of retroviral producer (Plat-E) cells 2 days after plasmid transfection detected by flow cytometry are shown. FIG. 2B, shows Lentiviral producer (293T) cells transfected with the indicated plasmid, and EGFP fluorescence detected by flow cytometry, 2 days after plasmid transfection.

FIG. 3 shows that Oct-4 and Sox2 core enhancer elements increase ES-specific expression of ETn promoter. FIG. 3A, shows the sequence of Oct-4 core enhancer element (CR4) and Sox2 core enhancer element (SRR2). Solid bar indicates Oct-4 or Sox2 transcript, and open box indicates exons. Known Sp1, Oct-4 and Sox2 binding site are indicated in the sequences. DE: distal enhancer, PE: proximal enhancer FIG. 3B, shows a schematic drawing of retroviral vectors, which have Oct-4 or Sox2 core enhancer elements. Oct-4 core enhancer element (CR4) or Sox2 core enhancer element (SRR2) were inserted as a concatamer into HSC1-pAMu-EGFP vector. Plus “(+)” indicates a forward orientation of the enhancer sequence; minus “(−)” indicates a reverse orientation of the enhancer sequence. FIG. 3C, CR4 and SRR2 enhancers increased the ES-specific expression of ETn promoter in mouse ES cells. Each retroviral vector was infected into J1 ES cells and NIH3T3 fibroblasts simultaneously, and 2 or 3 days after infection mean fluorescence of GFP positive populations were analyzed by flow cytometry. Graphs are an average from three different infections and error bars indicate standard deviation.

FIG. 4 shows that CR4 works as an ES-specific enhancer when inserted between EGFP and 3′LTR. FIG. 4A shows retroviral vector constructs which have enhancer elements 3′ of EGFP. FIG. 4B shows flow cytometry analysis 2 days after infection. Graphs are an average from three different infections; error bars indicate standard deviation. The CR4 enhancer (but not SRR2) increased the mean fluorescence in mES cells slightly when inserted between EGFP and 3′LTR.

FIG. 5 shows that Lentivirus-based EOS (ETn promoter with poly A site mutation, plus Oct-4/Sox2 binding sites.) vectors specifically express in mouse ES cells but not in mouse fibroblasts and extinguish upon in vitro differentiation. FIG. 5A, shows a schematic drawing of lentiviral vectors with several different internal promoters. The lentiviral vector backbone has a self-inactivating deletion in U3 of the 3′LTR so that EGFP is only expressed from an internal promoter. The nucleic acid of the start codon of EGFP is altered into a Kozak consensus sequence. cPPT: central polypurine tract, CTS: central termination signal, RRE: Rev responsible element. C(3+): trimer of CR4 enhancer element, S(4+), tetramer of SRR2 enhancer element. FIG. 5B, shows an EGFP fluorescence (right) and phase-contrast (left) microscopy shows specific expression of EOS lentivirus vectors in mouse ES cell colonies but not in surrounding MEFs (Mouse Embryonic Fibroblasts), whereas control PGK and EF1a lentivirus vectors express in both cell types. 1 or 10 microliters of concentrated lentiviruses were infected into mixed cultures of J1 mouse ES cells on MEFs that were not treated with mitomycin C. Images were taken at 2 days post-infection. FIG. 5C, shows flow cytometry demonstrates that EOS lentiviral vectors specifically express in mouse ES cells, and their expression levels and viral titers are higher than Oct-4 and Nanog promoters. Mixed cultures of mouse ES and MEF (no mitomycin C treatment) were infected with the indicated lentiviral vector and 2 days later EGFP expression was analyzed by flow cytometry. MEFs and mouse ES cells were separated by side-scatter (SSC), which correlates with complexity of cytoplasmic structure. MEFs with bigger cytoplasm have higher SSC value compared with mouse ES cells. “MEF only”—MEF without vector infection; “ES only”—mouse ES cells without vector infection. FIG. 5D shows that Lentiviral vectors comprising EOS-C(3+) and EOS-S(4+) constructs are active in mouse ES cells but not in MEFs. Lentiviral vectors were infected into mouse ES cells or MEFs separately and simultaneously and analyzed by flow cytometry 3 days after infection. Left panel: 200 μl of unconcentrated virus was infected into mES cells, middle panel: 10 μl of concentrated virus was infected into mES cells, right panel: 1 μl of concentrated virus was infected into MEF cells.

FIG. 6A, shows flow cytometry and fluorescent microscopy show that EOS lentiviral vectors (shown in FIG. 5A) expression in mouse ES cells is extinguished upon in vitro differentiation, whereas control PGK vector (high and low MOI) retains its expression. J1 mouse ES cells were infected with the indicated lentiviral vector and separated into two sets. One set was maintained as an ES culture (Mouse ES) and another set was differentiated by formation of embryoid bodies and dissociation as described in Methods section (Differentiated). Morphology of differentiated cells and their EGFP fluorescence are shown in right panels. FIG. 6B shows flow cytometry and fluorescent microscopy analysis show that EOS Eetroviral (HSC1 vector, shown in FIG. 3B) expression in mouse ES cells is extinguished upon in vitro differentiation, whereas control PGK vector retains its expression. FIG. 6C shows EOS-EGFP positive cells after differentiation showed continued ES-like colony morphology indicating that the vectors mark residual pluripotent cells that fail to differentiate. FIG. 6D shows EOS-EGFP expression was correlated with SSEA-1 expression during mouse ES cell differentiation. Mouse ES cells infected with lentiviral vectors were differentiated as described in Methods section and stained for the undifferentiated marker SSEA-1. PL-PGK-EGFP infected cells without primary (anti-SSEA1) antibody were used for EGFP single color compensation control (FL1, x-axis) and mock infected cells stained by SSEA-1 were used for PE-Cy5.5 single color compensation control (FL3, y-axis).

FIG. 7: shows that Lentivirus EOS vectors (described in FIG. 5A) are specifically expressed in human ES cells but not in human fibroblasts and are extinguished upon in vitro differentiation of human ES cells. FIG. 7A shows images of EGFP fluorescence specifically express EGFP from Lentivirus EOS vectors in human ES cell colonies but not in surrounding feeder cells, whereas control PGK and EF1a lentivirus vectors express in both cell types. CA-1 human ES cells cultured on feeders were infected with concentrated lentiviral vector. Images were taken 3 days postinfection and edges of human ES cell colonies are outlined. FIG. 7B shows flow cytometry and fluorescent microscopy analysis show that expression in human ES cells is extinguished upon in vitro differentiation, whereas control PGK and EF1a vectors retain their expression. CA-1 cells were differentiated with retinoic acid for 9 days and dissociated onto tissue culture plates. Three days after dissociation, differentiated cells were analyzed by fluorescence microscopy and flow cytometry. FIG. 7C, Lentivirus EOS vectors were not expressed in primary human dermal fibroblasts (HDFs); confirmed by flow cytometry and fluorescence microscopy. Primary HDFs were isolated and infected with 1 μl (flow cytometry) or 10 μl (microscope image) of concentrated lentiviral vectors. FIG. 7D shows flow cytometry results of 293 T (human embryonic kidney cell line), HeLa (human epithelial carcinoma cell line), and K562 (human erythromyeloblastoid leukemia cell line) were infected with 1 microliter of concentrated lentiviral vectors. Heavy line indicates experimental cell results; light line indicates mock infected negative control for each cell type.

FIG. 8 shows that expression of antibiotic resistance gene by EOS vector allows selective growth of undifferentiated ES cells. FIG. 8A shows retroviral vector based constructs which express neomycin resistance gene and EGFP under the control of PGK or EOS-C(3+) promoter. IRES: internal ribosome entry site. FIG. 8B shows infected mES cells and NIH3T3 cells that were mixed and selected by G418 at several concentrations. Six days after selection, the cells were fixed and stained for alkaline phosphatase activity.

FIG. 9 shows that the Lentivirus EOS vector “turns on” during reprogramming of mouse iPS cells and facilitates establishment of iPS cell colonies using puromycin (“puro”) selection. FIG. 9A shows an experimental outline of iPS cell induction. MEFs were divided into two groups, where one group was infected with EOS lenti vector encoding the EGFP and puromycin resistance genes. Twenty four hours later, each group was divided into 3 dishes (6×10⁵ cells/10 cm dish) for inducing iPS cells with either 4 factors (4F; Oct-4, Sox2, Klf4 and c-Myc) or 3 factors (3F; without c-Myc) or no factors (0F; mock infection). Next day, induced cells were transferred onto feeder cell plates with ES media. For the EOS infected group, puromycin selection was applied 7 days after induction. ES medium was changed daily and ES-like colonies were picked from 17 to 22 days after induction. FIG. 9B shows fluorescence microscopy of emerging iPS cell colony structure and activated EGFP expression from the EOS cassette (EOS-4F, EOS-3F) at 6 days post induction, whereas the mock infected plate (EOSOF) did not have any puromycin resistant colonies nor detectable EGFP expression. Most colonies were alkaline phosphatase positive coincident with EOS EGFP expression at day 14 after induction. FIG. 9C shows that EOS puromycin selection facilitates the formation of ES-like alkaline phosphatase positive colony numbers with 4 factor and 3 factor inductions, whereas the non-selected dish was covered with non-ES like cells. In addition, EOS puromycin selection facilitates the establishment efficiency of the 3 factor iPS cell lines from 3% (1 lines established our of 33 colonies picked) to 23.8% (5 lines extablished out of 21 colonies picked). WT-0F, no pluripotency factor control; WT-3F, 3-factor pluripotency factor treatment, no EOS vector; WT-4F, 4-factor pluripotency factor treatment, no EOS vector; EOS-4, 4-factor pluripotency factor treatment, infection with EOS vector; EOS-3, 3-factor pluripotency factor treatment, infection with EOS vector. FIG. 9D shows in vivo differentiation of mouse iPS cell lines into teratomas. Mouse iPS cell lines were injected into the testes of NOD/SCID mice and pathology performed after 5 weeks. Note that a single injection of each line gave rise to a teratoma indistinguishable from those derived from the mouse ES cell control. The teratoma contains a variety of typical structures from all three germ layers, such as neural tissue (ectoderm), cartilage (mesoderm), and ciliated epithelium (endoderm), showing in vivo pluripotency of the established mouse iPS cell lines.

FIGS. 10A-X show SEQ ID NO: 1-24, as described in Table 1.

FIG. 11 Flow cytometry and fluorescent microscopy show that EOS-EGFP expression in mouse iPS cells is extinguished upon in vitro differentiation, whereas some EOS3 #24 cells retain their EGFP expression and undifferentiated ES cell-like morphology. Simultaneously differentiated WT4 #1 iPS clone was used as a negative control for flow cytometry (thin line). Three independent differentiation experiments were performed and a representative result is shown. FIG. 11B EOS-EGFP positive EOS3 #24 cells formed significantly larger tumors than EOS-EGFP negative EOS3 #28 and #29 cells. Differentiated cells were injected into testes of NOD/SCID mice and the weight of testis with teratoma was measured 5 weeks after injection. Error bars indicate s.d. and asterisks (*) indicate P<0.05.

FIG. 12A shows a schematic illustration of an EGFP probe to detect EOS vectors integrated into mouse iPS cell lines; FIG. 12B shows a Southern blot analysis of three iPS cell lines. Isolated genomic DNAs were digested with BamHI, and integrated EOS vectors were detected by the EGFP probe. EOS3#24 and EOS3#28 cell lines have four copies of the vector integrated; EOS3#29 has two copies of the vector integrated in the genome.

FIG. 13A shows an experimental outline of human iPS cell induction. BJ human fibroblasts were infected with lentivirus encoding the ecotropic gammaretrovirus receptor and the bicistronic EOS lentiviral vector encoding EGFP and puromycin resistance genes. The cells were then infected with gammaretroviral vectors encoding the four human Yamanaka factors (4F; OCT4, SOX2, KLF4, c-MYC) and a pMXs-mRFP1 reporter to indicate the infection frequency. The cells were maintained for 1 week, then cultured on puro-resistant feeders in hES cell media. FIG. 13B, EOS-EGFP expression was detected by day 17 after induction, at which time puro selection (1 μg/ml) was applied to enrich for reprogrammed iPS cell colonies. FIG. 13C shows puromycin selection increases the percentage of TRA-1-60 and TRA-1-81 positive cells after 5 weeks of induction. SSEA-1 was used as a differentiation marker. The percentages were mean±s.d. from 3 independent induction plates. FIG. 13D, established human iPS cell lines (clone 4YA is shown as an example) co-express EOS-EGFP, NANOG, TRA-1-81 and SSEA-4 but have silenced the pMXs-mRFP1 gammaretroviral vector. FIG. 13E shows flow cytometry analysis of EOS-EGFP expression and hESC marker expression in established human iPA cell lines. CA-1 human ES cells were used as a positive control for TRA-1-60 and TRA-1-81 staining. FIG. 13F, Established human iPS cell line (clone 4YA) was differentiated by EB formation for 8 days and dissociated onto tissue-culture plates for 2 weeks. EOS-EGFP was extinguished (bottom panels) and became positive for lineage-committed cell markers, betaIII-tubulin (ectoderm, top-left panel), alpha-actinin (mesoderm, top-middle panel), and alpha-fetoprotein (endoderm, top-right panel). FIG. 13G, Human iPS cell line BJ-EOS 4YA formed mature teratomas that contain a variety of typical structures from all three germ layers, such as retinal and neural epithelia tissue (ectoderm, left panel), cartilage (mesoderm, middle panel), and ciliated gut-like epithelium (endoderm, right panel), confirming the in vivo pluripotency of the line established.

FIG. 14 shows EOS selection establishes Rett Syndrome-specific mouse and human iPS cell lines. FIG. 14A, PCR genotyping of MEFs from the Mecp2³⁰⁸ mouse model that expresses a truncated allele of Mecp2 identifies a heterozygous Mecp2^{+/308} (HET) embryo for reprogramming in comparison to Mecp2^{308/y} (MUT), control (WT) genotypes. FIG. 14B, phase-contrast (left) and EGFP fluorescence images (right) of Mecp2³⁰⁸ HET iPS cell line (HET-3F #1) shows ES-like colony morphology and activated EOS-EGFP expression. HET fibroblasts were infected with retroviruses expressing the three factors (3F; Oct-4, Sox2, Klf4) and EOS lentivirus. FIG. 14C, EOS-EGFP-expressing HET-3F #1 mouse iPS cell line stains positive for pluripotency markers Nanog and SSEA-1. FIG. 14D, Functional pluripotency of HET-3F #1 as revealed by positive staining for lineage-committed cell markers, betaIII-tubulin (ectoderm), alpha-actinin (mesoderm), and alpha-fetoprotein (endoderm) following EB-mediated in vitro differentiation. FIG. 14E, Sequencing of genomic DNA derived from a Rett Syndrome patient shows a heterozygous point mutation (from C to T) causing an R306C amino acid change in the transcriptional repression domain of MECP2. FIG. 14F, EOS-EGFP was activated in embryonic stem cell-like colonies during reprogramming (15 days post-induction). Scale bar: 50 μM. FIG. 14G, R306C human iPS cell line stains positive for pluripotency markers. Scale bar: 50 μm. FIG. 14H, Functional pluripotency of R306C human iPS cell line shown by differentiation into the three germ layers in vitro via embryoid body formation.

FIG. 15 shows the nucleotide sequence of HygroTK (SEQ ID NO: 40) as indicated in Table 2.

FIG. 16 shows the amino acid sequence of HygroTK (SEQ ID NO: 41) as indicated in Table 2.

FIG. 17 shows the nucleotide sequence of an EGFP-IRES-HygroTK construct (SEQ ID NO: 42).

### DETAILED DESCRIPTION

The present invention relates to nucleic acid sequences comprising regulatory sequences that direct expression of tag sequences in stem cells. The invention furthermore provides nucleic acid sequences that direct expression in embryonic or induced pluripotent stem cells.

The following description is of a preferred embodiment.

The present invention provides a nucleic acid construct comprising an pluripotent stem cell specific promoter sequence operatively linked to a tag sequence. Non-limiting examples of a pluripotent stem cell specific promoter sequence include a murine ETn (early transposon) element (SEQ ID NO: 1), or an ETn pAMu promoter (SEQ ID NO:2). The Etn pAMu promoter comprises a point mutation at position 183 of the ETn promoter, where an A is substituted for a T (this mutation is designated as: A183T). Other examples of pluripotent stem cell specific promoter sequences include promoter sequences from genes expressed in pluripotent stem cells, examples of such genes including, but not limited to, Oct-4 (Okumura-Nakanishi et al 2005), Nanog (Kuroda et al., 2005; Rodda et al., 2005), Sox2 (Tomioka et al., 2002), FGF-4 (Ambrosetti et al., 2000), Fbx15 (Tokuzawa et al., 2003), Utf1 (Nishimoto et al, 1999), Lefty1 (Nakatake et al, 2006), and Zfp206 (Wang et al., 2007), and Lin28.

An advantage of using a nucleic acid construct comprising, for example but not limited to, an ETn promoter sequence (SEQ ID NO: 1), or an ETn pAMu promoter sequence (SEQ ID NO: 2), operatively linked to a tag sequence to mark or select for iPS, is that standard protocols may be used, and similarly, the protocol is cell type neutral. Transfection of the construct may be carried out by any convenient or suitable method, and is the procedure is not dependent upon the transformation vector.

The nucleic acid construct according to some embodiments of the present invention may further comprise one or more than one enhancer sequence.

A tag sequence comprises one or more than one nucleic acid sequence encoding an amino acid sequence of interest. The amino acid sequence of interest may be a marker for color or fluorescence selection, for example, but not limited to GFP (Green fluorescent protein), EGFP (enhanced green fluorescent protein), Beta-galactosidase, luciferase, GUS, or the like (see, for example GUS Protocols: Using the GUS Gene as a Reporter of Gene Expression, S. R. Gallagher, Ed., Academic Press, Inc. (1992); Bronstein, I., et al. 1994. Anal. Biochem. 219:169-181; Alam, J. and Cook, J. L. 1990. Anal. Biochem. 188:245-254; WO 1997/042320; Nordeen, S. K. 1988. BioTechniques 6:454-457). The amino acid sequence of interest may be an antibiotic, drug or toxin resistance gene product, for example but not limited to puromycin-N-acetyl-transferase (confers resistance to puromycin; Vara et al. Nucl. Acids Res. 1986; 14: 4617-4624), aminoglycoside 3′ phosphotransferase (produced by the neo gene of Tn5, and confers resistance to G418 and neomycin; U.S. Pat. No. 4,784,949, or other antibiotic or toxin-resistance gene product, or the like, that allows cells expressing the amino acid sequence to survive and grow in the presence of the antibiotic, drug or toxin. SEQ ID NO's: 23 and 24 provide non-limiting examples of tag sequences. The amino acid sequence of interest may be an enzyme which catalyses the conversion of a non-toxic prodrug into a toxic form (so called suicide gene), for example but not limited to thymidine kinase gene, cytosine deaminase, cytochrome P450, nitroreductase, carboxypeptidase G2, purine nucleoside phosphorylase or the like (see, for example “Suicide genes for cancer therapy.” Portsmouth D. et al., Mol Aspects Med. 2007; 28:4-41), that allows cells expressing the amino acid sequence to kill or stop growing in the presence of the drug (“negative selection”). The amino acid sequence of interest may be a cell-surface antigen which can be recognized by a specific antibody (“cell-surface selection”)

A tag sequence may be assayed, for example, using colorimetric assays, drug selection assay, FACS analysis (Shapiro, H. M. 1988. Practical Flow Cytometry, 2nd ed Wiley-Liss, New York), CELISA, ELISA (Lequin R, 2005. Clin. Chem. 51: 2415-8), western blot (Burnette, W. N. 1981. Anal. Biochem. 112:195-203), Northern blot, Southern blot, PCR (Saiki, R. K. et al. 1988. Science 239:487-491), RT-PCR (Frohman, M. A., Dush, M. K., and Martin, G. R. 1988. Proc. Natl. Acad. Sci. U.S.A. 85:8998-9002), or the like. The nucleic acid transcript of the tag sequence may also be assayed by RT-PCR, northern blotting (Alwine et al 1977. Proc. Natl. Acad. Sci 74:5350), Southern blotting (Southern, E M. 1975. J. Mol Biol. 98:503), 5′ RACE, 3′RACE, sequencing (Sanger F, et al. Proc Natl Acad Sci USA. 1977. 74:5463-7; Maxam A M, Gilbert W., Proc Natl Acad Sci USA. 1977. 74:560-4), or other sequence-based assays.

Tag sequences comprising more than one nucleic acid sequence encoding an amino acid sequence of interest may further comprise a nucleotide sequence to facilitate expression of more than one nucleic acid sequence, for example, a nucleotide sequence comprising an internal ribosome entry site (IRES). Examples of IRES sequences are taught in, for example, Baird S D et al., RNA. 12:1755-85 2006). Another example of such a tag sequence may comprise a sequence encoding enhanced green fluorescent protein (EGFP) operatively linked to an IRES and a sequence encoding an amino acid sequence that confers resistance to antibiotics, such as puromycin (PuroR). When the nucleic acid is transcribed, all three sequences are produced as a single RNA transcript. When the RNA transcript is translated, ribosomes recognize both the 5′ cap (a translation initiation signal) and the IRES, and translation proceeds in a normal manner. A cell that comprises and expresses such a construct is therefore identifiable both by fluorescence, and by growth on culture medium comprising puromycin (“puro”). Other examples of such nucleic acids may comprise neomycin phosphotransferase (confers neomycin resistance) or hygromycin phosphotransferase (confers hygromycin resistance) in place of puromycin phosphotransferase (confers puromycin resistance). Other examples of such nucleic acids may comprise beta-galactosidase in place of the EGFP

Another example of a tag sequence according to some embodiments of the invention includes a nucleic acid comprising a nucleotide sequence encoding a fusion protein, such as beta-geo, HygroTK or NeoEGFP. A nucleotide sequence encoding beta-geo comprises a sequence encoding beta-galactosidase fused in-frame with a sequence encoding a protein for neomycin phosphotransferase. A nucleotide sequence encoding HygroTK comprises a sequence encoding a protein for hygromycin phosphotransferase fused in-frame with a sequence encoding thymidine kinase. A nucleotide sequence encoding NeoEGFP comprises a sequence encoding a protein for neomycin phosphotransferase fused in-frame with a sequence encoding enhanced green fluorescent protein. Another example of a tag sequence may include a nucleic acid comprising two or more nucleotide sequences encoding amino acid sequences of interest and connected by a nucleotide sequence encoding a cleavage peptide, such as a Picornavirus 2A peptide, or a 2A-like peptide (Szymczak A L et al., Nat Biotechnol. 2004). Such tag sequences may be expressed from a single promoter of a vector comprising the nucleic acid, and the translated polypeptide self-cleaved into the individual amino acid sequences of interest. Examples of such amino acid sequences include, but are not limited to, pluripotency factors, beta galactosidase, hygromycin phosphotransferase, neomycin phosphotransferase, thymidine kinase, green fluorescent proteins and

A tag sequence may also express other factors in a cell, for example one or more than one pluripotency factor, or one or more than one factor for expression in a developmental-stage specific manner. Examples of such pluripotency factors include, but are not limited to Oct-4, Nanog, Sox2, FGF-4, Fbx15, Utf1, Lefty1, Klf-4, c-Myc, Lin28 or Zfp206 (see references supra.). Pluripotency factors may also include various compounds, agents, proteins, peptides or other molecules that may be transfected into, or added to the culture medium of a cell to be induced to pluripotency, or to maintain pluripotency in a cell as would be known to one of skill in the art.

By “operatively linked” it is meant that the particular sequences, for example a promoter or enhancer, and a coding region of interest, interact either directly or indirectly to carry out an intended function, such as mediation or modulation of gene expression. The interaction of operatively linked sequences may, for example, be mediated by proteins that interact with the operatively linked sequences. Additionally, an IRES may be operatively linked to a nucleic acid sequence facilitating translation of the nucleic acid.

The relative position of any two or more operatively linked elements may be described as “in cis” or “in trans”. Elements that are in cis are found on the same molecule, for example encoded by the same nucleic acid, e.g. a vector. Elements that are in trans are found on two or more separate molecules. The relative position of two or more elements in cis may also be described as being upstream (5′) or downstream (3′) from an element. Elements in cis may be adjacent, or may be separated by one or more other elements, for example a tag sequence, or a regulatory element such as a promoter, another enhancer, a termination signal or the like.

An enhancer sequence (may also be referred to as a motif) is a nucleic acid sequence or region of DNA that aids in the transcription of a gene or nucleotide sequence. An enhancer sequence may be located at a distance from the nucleotide sequence being transcribed, for example, it may be located on a separate chromosome, or on a separate nucleic acid molecule. Enhancer sequences may be located 5′ or 3′ to the nucleotide sequence being transcribed, and may function in either ‘orientation’ (either positive “+”, or negative “−” orientation). Enhancer sequences may also be 5′ or 3′ relative to the promoter directing transcription of a coding sequence of the nucleotide sequence being transcribed. These variants may be combined in a single construct to modify transcription of the coding sequence.

An “enhancer unit” may comprise one or more than one enhancer sequences, or motifs. Each of the one or more than one enhancer sequence may function constitutively, function in a developmental stage or tissue specific manner, or function constitutively and in a developmental stage or tissue-specific manner. An enhancer sequence, or an enhancer unit, selective for, for example, an embryonic stem cell stage, may be combined with the ETn promoter to obtain an increase in expression of a tag sequence. An increase in expression of the tag sequence can be determined by comparing the level of expression of the tag sequence (or coding sequence product) that is modified by an enhancer sequence or an enhancer unit, to the level of expression of the tag sequence (or coding sequence product) obtained in the absence of the enhancer sequence or enhancer unit. Enhancer sequences may be obtained from genes expressing in ES cells. Examples of such genes include, but are not limited to, Oct-4 (Okumura-Nakanishi et al 2005), Nanog (Kuroda et al., 2005; Rodda et al., 2005), Sox2 (Tomioka et al., 2002), FGF-4 (Ambrosetti et al., 2000), Fbx15 (Tokuzawa et al., 2003), Utf1(Nishimoto et al, 1999), Lefty1 (Nakatake et al, 2006), and Zfp206 (Wang et al., 2007).

An enhancer sequence may include a CR4 element from Oct-4 (SEQ ID NO: 3), an SRR2 element from Sox2 (SEQ ID NO: 4), a CR4 element in reverse orientation (SEQ ID NO: 5), a SRR2 element in reverse orientation (SEQ ID NO: 6), or a combination thereof.

An enhancer unit may comprise one or more than one enhancer sequence. An enhancer sequence or motif may be 5′ to a promoter, or 3′ to a promoter. For example, a nucleic acid may comprise a promoter operatively linked to a tag sequence, and have an enhancer motif located 5′ relative to the promoter, 3′ relative to the promoter and 5′ relative to the tag sequence, 3′ relative to both the promoter and tag sequence, or a combination thereof. Sequences comprising an enhancer unit may be all in a positive orientation, all in a negative orientation, or a combination of both positive and negative orientation. If the enhancer unit comprises two or more enhancer sequences of both positive and negative orientation, the most-upstream sequence may have a positive or negative orientation. Orientation of the enhancer sequence does not need to relate or correspond to the order or position of the enhancer sequence within the enhancer motif.

SEQ ID NOS: 7, 8, 11 and 12 are non-limiting examples of nucleic acid constructs comprising one enhancer sequence (SRR2 or CR4) located 5′ to a promoter sequence (ETn pAMu) in a forward (SEQ ID NOS: 7 and 11) or reverse (SEQ ID NOS: 8, 12) orientation.

SEQ ID NOS: 9, 10, 13 and 14 are non-limiting examples of nucleic acid constructs comprising more than one enhancer sequence (SRR or CR4) located 5′ to a promoter sequence (ETn pAMu) in a forward (SEQ ID NOS: 9, 13 and 14) or reverse (SEQ ID NO: 10) orientation.

SEQ ID NOS: 15-18 are non-limiting examples of nucleic acid constructs comprising one enhancer sequence (CR4 or SRR2) located 3′ to a nucleic acid sequence encoding a tag sequence (EGFP), in the forward (SEQ ID NOS: 15, 17) or reverse (SEQ ID NOS: 16, 18) orientation.

SEQ ID NOS: 19-22 are non-limiting examples of nucleic acid constructs comprising one or more than one enhancer sequence located 5′ to a promoter sequence (ETn pAMu) and located 3′ to a tag sequence (EGFP). The enhancer sequences may be in the forward or reverse orientation.

Therefore, the present invention provides for a nucleic acid comprising an ETn poly A mutated (pAMu) promoter sequence (SEQ ID NO: 2) operatively linked to a tag sequence and one or more than one enhancer sequence, the one or more than one enhancer sequence active in a pluripotent stem cell. The one or more than one enhancer sequence may be selected from one or more than one CR4 enhancer sequences, two or more than two SRR2 enhancer sequences, or a combination thereof. The enhancer motif may be located upstream of the pAMu promoter sequence.

In some embodiments of the invention, the one or more than one CR4 enhancer sequence may be selected from SEQ ID NOS: 3 and 5. In some embodiments of the invention, the one or more than one SRR2 enhancer sequence may be selected from SEQ ID NOS: 4 and 6.

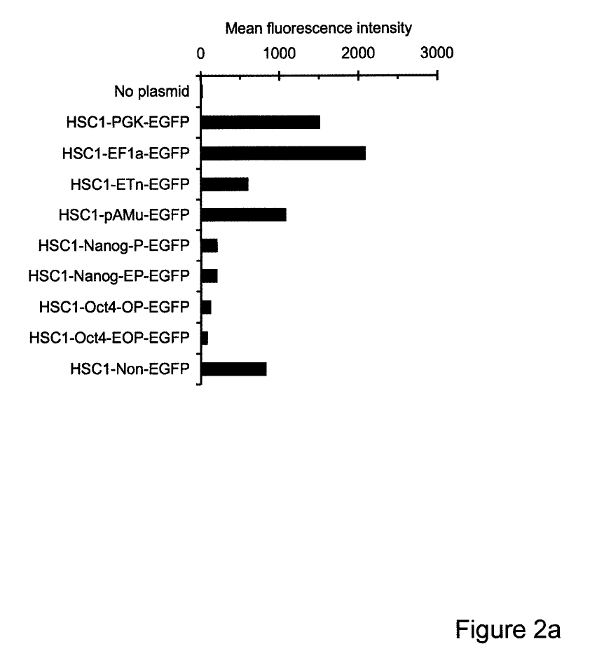

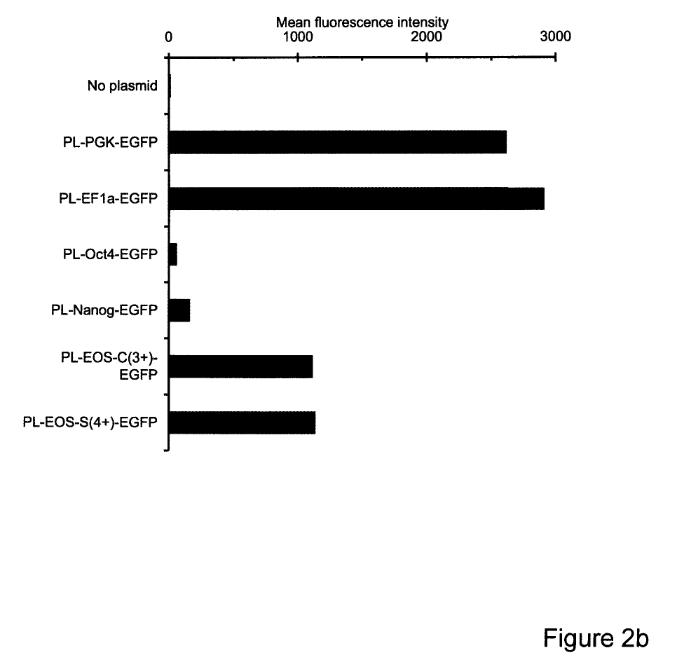

Therefore, the invention provides a nucleic acid construct comprising an ETn pAMu promoter sequence and one or more than one enhancer sequence. The one or more than one enhancer sequence may be in a forward or reverse orientation, located 5′ to the promoter, 3′ to the promoter, 5′ and, 3′ to the promoter, or a combination thereof. The one or more than one enhancer sequence in the nucleic acid construct may be the same, or may be different. Non-limiting examples of such constructs may be found with reference to FIGS. 2-4.

The present invention also provides for a nucleic acid comprising an ETn poly A mutated (pAMu) promoter sequence (SEQ ID NO: 2) operatively linked to a tag sequence and operatively linked to one, two, three, four or more SRR2 enhancer sequences, the enhancer sequences may be located 5′ to the ETn pAMu promoter.

The present invention also provides for a nucleic acid comprising an ETn poly A mutated (pAMu) promoter sequence (SEQ ID NO: 2) operatively linked to a tag sequence, and operatively linked to one, two, three, four or more CR4 enhancer sequences in a positive orientation and may be located 5′ to the pAMu promoter.

The present invention also provides for a nucleic acid comprising an ETn poly A mutated (pAMu) promoter sequence (SEQ ID NO: 2) operatively linked to a tag sequence, and operatively linked to one, two, three, four or more CR4 enhancer sequences in a negative orientation, and located 5′ to the pAMu promoter.

The present invention also provides a method of identifying an embryonic stem cell (ES). To identify an ES cell, the nucleic acid comprising an ETn sequence operatively linked to a tag sequence is transfected into a cell or population of cells. The cells are grown in suitable medium and assayed for expression of the tag sequence, or the presence or expression of the amino acid sequence encoded by the tag sequence, where the detected of the tag sequence or amino acid sequence encoded by the tag protein identifies the ES cell.

The present invention also provides a method of identifying an induced pluripotent stem cell (iPS). To identify an iPS cell, a population of cells comprising the iPS is transfected with a nucleic acid comprising an ETn or ETn pAMu promoter or an ETn pAMu promoter operatively linked to one or more than one enhancer sequence, and directing expression of a tag sequence. The transfected cells are grown under suitable conditions and the cells expressing the tag sequence are selected.

The method of selection of a tag sequence in the above methods, will be dependent on the tag sequence used. For example, if the tag sequence provides for expression of EGFP, the iPS cells may be selected by FACS analysis (see, for example, Shapiro, H. M. 1988. Practical Flow Cytometry, 2nd ed Wiley-Liss, New York). Use of a fluorescent marker such as EGFP (thus enabling use of FACS) provides an additional advantage of separating out the iPS from the remainder of the population of cells, enriching for iPS. Positive selection may also be used. As an example, the tag sequence may encode an amino acid sequence of interest that provides resistance (for example a puromycin resistance enzyme) to an agent in the culture medium (for example puromycin). Cells that express the enzyme will continue to grow in the presence of puromycin, while those that do not express the enzyme (those that are not at a developmental stage where the tag sequence is expressed) will die.

The present invention also provides a method of identifying or killing a residual undifferentiated cell following induced differentiation. To identify a residual undifferentiated cell, a nucleic acid comprising an ETn or ETn pAMu sequence operatively linked to a tag sequence is transfected into a cell or population of cells. The cells are differentiated into suitable cell types and assayed for expression of the tag sequence to identify or to negatively select an undifferentiated cell.

The tag sequence as described herein may be one or more negative selection markers, for example a suicide genes which catalyse the conversion of a non-toxic prodrug into a toxic form, for example but not limited to thymidine kinase (or a nucleic acid comprising a nucleotide sequence encoding thymidine kinase), cytosine deaminase, or cytochrome P450, or the like (see for example Portsmouth D. et al., Mol Aspects Med. 2007; 28:4-41; herein incorporated by reference), that allows cells expressing the amino acid sequence to kill or to stop growing in the presence of a pharmaceutical agent drug (such as ganciclovir, or acyclovir). An advantage to performing such a negative selection may include the reduction or removal of potential teratoma-forming cells in the population.

Examples of such directed differentiation methods or procedures include using a defined set of growth factors added to the cell media to induce differentiation into specific cell lineages such as neural, cardiac, or pancreatic cells. Chemicals, cytokines or hormones such as Retinoic Acid, or the introduction of lineage-specifying master genes (for example MyoD to induce muscle) that direct differentiation into a specific lineage or cell type may also be used.

Negative selection may also be used in combination with other selection criteria to identify selected cells. For example, tag sequences encoding both a drug resistance enzyme (e.g. a sequence encoding a puromycin resistance enzyme) and a negative selection element (e.g. a sequence encoding thymidine kinase) may be transfected into the cells. Reprogrammed cells may be selected for by growing in puromycin. During a subsequent directed differentiation method or procedure, gancyclovir may be added to the growth medium to select against cells expressing thymidine kinase (e.g. those that did not undergo subsequent directed differentiation). An advantage to performing such a multi-step selection may include reduction or removal of potential teratoma-forming cells in the population, and be of particular interest if the iPS cells are to be used in a subject.

Alternately, negative selection may be employed in vivo post-transplantation. A subject may be administered a population of cells, or tissue comprising such cells, the cells having previously been transfected with a nucleic acid comprising a negative selection element, directed to differentiate to the desired cell or tissue type. Once the cells or tissue are transplanted or administered to the subject, the subject may be administered a pharmaceutical agent or drug to select against any undifferentiated cells. For example, if the negative selection element is a nucleic acid encoding thymidine kinase operatively linked to a promoter sequence that is selectively expressed in an iPS cell, any undifferentiated iPS cells will be killed following administration of a course of, for example, gancyclovir, or acyclovir to the subject.

Transfection refers generally to the introduction of foreign material, frequently nucleic acid, into a cell, such as a mammalian cell. Transfection of a cell frequently results in a change in one or more properties of the cell, for example, expression of a foreign transcript or protein, alteration in growth pattern, or the like. Cells may be transfected by any of several methods known in the art, for example use of calcium phosphate (Graham F L, van der Eb A J, Virology. 1973 52(2):456-467); use of dendrimers to bind the nucleic acid and enhance uptake; liposomal transfection (Sells, M. A., Li., J., and Chernoff, J. 1995. BioTechniques 19:72-78); transfection using cationic polymers such as DEAE-dextran, polyethylenimine or poly-L-ornithine (Scangus G and Ruddle F H. 1981 Gene 14:1-10); ‘gene gun’ or biolistic particle delivery (U.S. Pat. No. 4,956,050, U.S. Pat. No. 5,204,253, U.S. Pat. No. 6,194,389); nucleofection (Aluigi M et al 2006. Stem cells 24:454-461); electroporation; heat shock; magnetofection (Plank C et al 2003. Biol. Chem 384:737-47; U.S. Pat. No. 5,547,932); or transfection using viral vectors, such as retroviral or lentiviral vectors (Wilson et al., 1990. PNAS 87:439-443, Kasid et al., 1990). Protocols for such methods and techniques may be found in, for example, Current Protocols in Molecular Biology (Ausubel et al., Editors. Wiley Interscience 2008).

Cells may be stably or transiently transfected. If the transfected nucleic acid is to persist in daughter cells following mitosis or meiosis, stable transfection is preferable. The transfected nucleic acid may be co-transfected with another gene that provides a selective advantage, such as resistance to a drug or agent (where the drug or agent is added to the cell culture medium following transfection), or ability to survive in the absence of a particular metabolite. The transfected nucleic acid may also be co-transfected with another gene that increase the transfection efficiency (such as a DNA binding protein which has a cell-permeabilization signal), increase the chromosomal integration efficiency (such as an integrase or a transposase), increase the targeting into a specific locus of chromosome (such as a zinc-finger protein which bind to a specific DNA sequence, or a site-specific endonuclease), facilitate homologous recombination, or maintain nucleic acid as an episome (such as SV40 large T antigen or Epstein-Barr Virus nuclear antigen 1). Examples for such methods and techniques may be found in, for example, Palazzoli F. et al., Current Gene Therapy, 2008. “Transduction”, “infection” (in reference to transfection using a viral vector, such as a retroviral vector), “infection by transformation” are other terms that may be used interchangeably with transfection, in reference to the introduction of foreign material such as nucleic acid into a cell, and the systems that facilitate such introduction.

Viral vectors, such as retroviral vectors, for example but not limited to gammaretroviral or lentiviral vectors, are one option available for introducing foreign nucleic acid into cells, in particular primary fibroblasts, ES or iPS cells. Other examples of viral vectors include, but are not limited to adenovirus vectors, parvovirus vectors, herpesvirus vectors, adeno-associated virus vector, poxivirus vectors, or the like. Nucleic acids according to some embodiments of the invention may be incorporated in a retroviral vector for delivery to the cells. The silencing or attenuation of nucleotide sequences may be observed following retroviral vector transfection. This silencing may include tag sequences. As shown in the examples, silencing may be overcome through the use of an ETn or ETn pAMu promoter, or an ETn pAMu promoter operatively linked to one or more than one enhancer sequence, in the vector to direct transcription of the tag sequence. Thus, a method to overcome silencing of genes or nucleotide sequences following retroviral transfection is provided. The method comprises transfecting an adult fibroblast or embryonic stem cell with a vector comprising an ETn or an ETn pAMu promoter operatively linked to a tag sequence, and expressing the tag sequence.

A retroviral or lentiviral vector that is self-inactivating (SIN) may also be suitable for introducing foreign nucleic acid into cells. An example of a self-inactivating retroviral vector is HSC-1 (Osborne et al., 1999). HSC1 retroviral vector has a self-inactivating deletion in 3′LTR U3 and do not contain any known ES-specific silencer binding sites. After reverse transcription and integration, the self-inactivating (SIN) deletion is copied into 5′LTR so that EGFP is only expressed from an internal promoter.

Transposon vectors may also be used to introduce foreign nucleic acid into cells, including primary fibroblasts, ES or iPS cells. Examples of transposon vectors include piggyBac (Cary, L. C. et al., 1989. Virology 172: 156-169; Fraser, M. J., L. et al., 1995. Virology 211:397-407) or the Sleeping Beauty Transposon™ System (SBTS) (U.S. Pat. No. 6,489,458).

The present invention, further provides for a method of identifying an iPS comprising transfecting a cell with a viral vector comprising one or more pluripotency factors, transfecting the cell with a nucleic acid comprising an ETn promoter sequence (SEQ ID NO: 1) or an ETn pAMu promoter sequence (SEQ ID NO: 2), growing the cell, and selecting for the iPS.

Also provided by the present invention is a cell comprising a nucleic acid comprising an ETn promoter sequence (SEQ ID NO: 1) or an ETn pAMu promoter sequence (SEQ ID NO: 2) operatively linked to a tag sequence. The cell may be an iPS, or may be an ES from a subject.

Also provided by the present invention is a method of producing an induced pluripotent stem cell. This method comprises transfecting a cell with one or more than one pluripotency factors to produce a pluripotent cell, transfecting the pluripotent cell with a nucleic acid comprising one or more than one regulatory sequence that direct expression of tag sequences in a stem cell to produce a transfected cell, growing the transfected cell, and selecting for an induced pluripotent stem cell.

The present invention also provides another method of producing an induced pluripotent stem cell. This method comprises transfecting a cell with a nucleic acid comprising regulatory sequences that direct expression of tag sequences in a stem cell to produce a transfected cell; transfecting the transfected cell with one or more than one pluripotency factor to produce a pluripotent cell, growing the pluripotent cell, and selecting for an induced pluripotent stem cell.

The methods of Takahashi et al., 2006, Okita et al., 2007 or Takahashi et al., 2007 may be employed, for example, to induce pluripotent stem cells from fibroblast cultures. Alternately, the methods of Okita et al., 2008 or Stadtfeld et al., 2008 may be employed to induce pluripotent stem cells from differentiated, or partially differentiated cell cultures.

Other methods of introducing pluripotency factors to a cell to induce reprogramming to an iPS state may also be used. For example, pluripotency factors may be introduced using vectors other than viral vectors (e.g. episomal nucleic acid), or by transfection of the pluripotency factors themselves directly into the cell. The pluripotency factors may further comprise amino acid motifs or domains that facilitate entry of a protein into a cell, for example, protein transduction domains. Protein transduction domains may be fused, bound or coupled to a pluripotency factor. Examples of protein transduction domains include HIV TAT, cell-penetrating peptides, antennapedia protein transduction domain, polyarginine oligomers, polylysine oligomers, KALA, MAP, transportan, PTD-5 or the like (see, for example, Kabouridis 2003. Trends in Biotechnology 21:498-503). Other methods may further include chemical induction of pluripotency (e.g. “chemical reprogramming”) by addition of small molecules that mimic pluripotency factors, activate pluripotency factors, or enhance reprogramming efficiency to the culture medium.

For example, a cell reprogrammed to an iPS state may further have nucleic acids according to some embodiments of the invention, or vectors comprising such nucleic acids delivered concurrently with, a vector or vectors for reprogramming, or subsequent to the reprogramming. Alternately, reprogramming may be facilitated by any method, including chemical reprogramming (e.g. addition of small molecules that mimic pluripotency factors or enhance reprogramming efficiency directly to the culture medium) or transient methods of delivering pluripotency factors to the cells (e.g. using adenovirus vectors, plasmid vectors, episomes, tat-fusion proteins or the like). The resulting iPS cells may be used, for example, to generate new lung, heart or bone tissue for patient-specific personalized regenerative medicine. For example, US Patent Publications 2004/0072343, 2007/00207759, 2007/0025973, 2003/0211603, 2007/0196918 disclose various methods of reprogramming differentiated, or partially differentiated cells.

Other methods of reprogramming to convert a cell into a pluripotent state may also be used. For example, nuclear transfer from a somatic cell into a pluripotent cell or into an oocyte (Yang X. et al., nature Genetics, 2007), or cell-cell fusion of a somatic cell and a pluripotent stem cell (Cowan et al., Science, 2005), where the somatic cell comprising one or more than one regulatory sequence that direct expression of tag sequences in a stem cell to produce a tranfected cell.

The nucleic acid may furthermore comprise sequences that direct expression in embryonic or induced pluripotent stem cells, examples of such sequences include ETn (SEQ ID NO: 1) and ETn pAMu (SEQ ID NO: 2). The nucleic acid may further comprise enhancer sequences, such as those selected from the group comprising SEQ ID NO: 3, 4, 5 or 6; the tag sequence may encode an amino acid sequence of interest.

These methods may be applied to any adult cell that may be reprogrammed to an ES-like stage, for example, an iPS. Examples of adult cells include, but are not limited to, fibroblasts, cynoviocytes (Takahashi et al., Cell, 2007), mesenchymal stem cells (Park et al., Nature, 2007), hepatocytes, keratinocytes, neural stem cell or neural progenitor cell, skin progenitor cell, epiblast derived stem cell, or gastric epithelial cells (Aoi et al., Science, 2008).

By the term “subject”, it is meant an organism, from whom cells may be isolated, or to whom cells according to some embodiments of the invention, may be administered. Examples of a subject include, but are not limited to, humans, primates, birds, swine, sheep, horse, dogs, cats, livestock, rabbits, mice, rats, guinea pigs or other rodents, and the like. Such target organisms are exemplary, and are not to be considered limiting to the applications and uses of the present invention.

Cells that have been selected on the basis of the tag sequence may be further characterized to determine the insertion point of the transgenes, or other characteristics as may be suitable for the desired application of the cells, including modeling human disease states or for therapeutic applications. For example, iPS cells may be generated from cells obtained from a subject or animal model of a disease or disorder having one or more genetic components, and these iPS cells used to generate a renewable source of cells or tissue demonstrating a particular characteristic or phenotype found in cell or tissue of the affected subjects or animal models. Such a renewable source of cells or tissue may be used to study the defects that underlie the particular disease or disorder and for evaluating the role of various genes in this process, for example, via rescue experiments or drug screenings. Examples of such diseases or disorders include any genetic disorder or congenital defect, such as, but not limited to, various neural defects having a genetic component (e.g. autism, Rett syndrome, schizophrenia), cystic fibrosis, various cardiac defects having a genetic component (e.g. Hypertrophic Cardiomyopathy (HCM), Marfan Syndrome, Long QT Syndrome, DiGeorge Syndrome), various musculoskeletal disorders having a genetic component (e.g. Muscular Dystrophy, Marfan Syndrome), Progeria, various cancers, or the like.

In addition cells according to some embodiments of the invention may be used to repair, regenerate or replace damaged tissue, such as lung, heart, or bone tissue for subject-specific regenerative medicine.

The invention also provides for a kit for identifying a pluripotent stem cell or an embryonic stem cell, comprising a nucleic acid comprising an ETn pAMu promoter sequence operatively linked to a tag sequence, and instructions for its use. The kit may further comprise one or more than one pluripotency factor, media, one or more than one other agents useful in selecting a pluripotent stem cell, or a combination thereof. The kit may further provide nucleic acids comprising a sequence encoding one or more than one pluripotency factor, such as Oct4, Sox2, Klf4, c-Myc or a combination thereof, and may further comprise transfection reagents for transfecting a cell. Instructions for use of the nucleic acids as described herein, transfection reagents or instructions for transfecting a cell, as well as instructions for screening for iPS cells as described herein may also be provided in such a kit.

The ability of EOS vectors to mark, enrich and maintain ES and iPS cell lines makes such vectors useful as reporters to aid in increasing the efficiency of isolating reprogrammed iPS cell lines from transgenic animals or from patient biopsies to model disease in vitro. Further, EOS vectors may be used to optimize reprogramming technologies by aiding in quantification and/or isolation of induced cells at the appropriate developmental stage.

The nucleic acid constructs and vectors provided by the present invention further allow for constant selection to maintain and expand iPS cells in a pluripotent state. It has been previously demonstrated that, in the context of in vitro iPS cell applications, retroviral or lentiviral integrations do not hinder disease-specific iPS cell line generation, nor do they influence phenotyping of affected cell types (Park et al., 2008; Dimos et al., 2008). The ability of EOS vectors to be imaged for EGFP expression or selected for puromycin resistance may be useful and valuable attributes for optimizing novel reprogramming technologies employing transient factor delivery methods or using high-throughput screens of small molecules. Directed differentiation procedures may need to be optimized for each disease-specific iPS cell line generated, and EOS vector expression may be used to monitor the numbers of responding, or non-responding pluripotent stem cells in this context.

Sequences according to various embodiments of the invention are described in Tables 1 and 2 and in the figures and accompanying text of the specification.

TABLE 1
Sequence table

| | | Figure |
|---|---|---|
| SEQ ID NO: | Description | reference |
| 1 | WT ETn type II #6 LTR promoter region (ETn) | 10A |
| 2 | Poly A mutated ETn type II #6 LTR promoter region (ETn | 10B |
| | pAMu) | |
| 3 | CR4 - Conserved region 4 in positive orientation. | 10C |
| 4 | SRR2 - Sox Regulatory Region 2 in positive orientation | 10D |
| 5 | CR4 in negative orientation | 10E |
| 6 | SRR2 in negative orientation) | 10F |
| 7 | (EOS-C(+); (HSC1-CR4(+)-pAMu-EGFP) has one CR4 | 10G |
| | enhancer sequence in the forward orientation, 5′ to the ETn | |
| | pAMu promoter. | |
| 8 | HSC1-C(−)-pAMu-EGFP) has one CR4 enhancer sequence in | 10H |
| | the reverse orientation, 5′ to the ETn pAMu promoter. | |
| 9 | (EOS-C(3+); PL-EOS-C(3+)A-EiP) has three CR4 enhancer | 10I |
| | sequences in the forward orientation, 5′ to the ETn pAMu | |
| | promoter. | |
| 10 | EOS-C(3−); HSC1-C(3−)-pAMu-EGFP) has three CR4 | 10J |
| | enhancer sequences in the reverse orientation, 5′ to the ETn | |
| | pAMu promoter. | |
| 11 | (EOS-S(+); HSC1-S(+)-pAMu-EGFP) has one SRR2 enhancer | 10K |
| | sequence in the forward orientation, 5′ to the ETn pAMu | |
| | promoter. | |
| 12 | HSC1-S(−)-pAMu-EGFP) has one SRR2 enhancer sequence in | 10L |
| | the reverse orientation, 5′ to the ETn pAMu promoter | |
| 13 | (EOS-S(2+); HSC1-SRR2(2+)-pAMu-EGFP) has two SRR2 | 10M |
| | enhancer sequences in the forward orientation, 5′ to the ETn | |
| | pAMu promoter. | |
| 14 | (EOS-S(4+); PL-EOS-S(4+)A-EiP) has four SRR2 enhancer | 10N |
| | sequences in the forward orientation, 5′ to the ETn pAMu | |
| | promoter. | |
| 15 | (HSC1-pAMu-EGFP-CR4(+); pAMu-EGFP-CR4(+)) has one | 100 |
| | CR4 enhancer sequence in the forward orientation, 3′ to the tag | |
| | sequence EGFP (underlined). | |
| 16 | (HSC1-pAMu-EGFP-CR4(−); pAMu-EGFP-CR4(−)) has one | 10P |
| | CR4 enhancer sequence in the reverse orientation, 3′ to the tag | |
| | sequence EGFP (underlined). | |
| 17 | (HSC1-pAMu-EGFP-SRR2(+); pAMu-EGFP-SRR2(+)) has | 10Q |
| | one SRR2 enhancer sequence in the forward orientation, 3′ to | |
| | the tag sequence EGFP (underlined). | |
| 18 | (HSC1-pAMu-EGFP-SRR2(−); pAMu-EGFP-SRR2(−)) has | 10R |
| | one SRR2 enhancer sequence in the reverse orientation, 3′ to | |
| | the tag sequence EGFP (underlined). | |
| 19 | (HSC1-C(3+)-pAMu-EGFP-S(−); C(3+)-pAMu-EGFP-S(−)) | 10S |
| | has three CR4 enhancer sequences in the forward orientation, | |
| | 5′ to the pAMu promoter and one SRR2 enhancer sequence in | |
| | the reverse orientation, 3′ to the tag sequence EGFP | |
| | (underlined). | |
| 20 | (HSC1-C(3+)-pAMu-EGFP-S(2+); | 10T |
| | C(3+)-pAMu-EGFP-S(2+)) has three CR4 enhancer sequences | |
| | in the forward orientation, 5′ to the pAMu promoter and two | |
| | SRR2 enhancer sequences in the forward orientation, 3′ to the | |
| | tag sequence EGFP (underlined). | |
| 21 | (HSC1-S(2+)-pAMu-EGFP-C(+); | 10U |
| | HSC1-S(2+)-pAMu-EGFP-C(+)) has two SRR2 enhancer | |
| | sequences in the forward orientation, 5′ to the pAMu promoter | |
| | and one CR4 enhancer sequence in the forward orientation, 3′ | |
| | to the tag sequence EGFP (underlined). | |
| 22 | (HSC1-S(2+)-pAMu-EGFP-C(2−); S(2+)-pAMu-EGFP-C(2−)) | 10V |
| | has two SRR2 enhancer sequences in the forward orientation, | |
| | 5′ to the pAMu promoter and two CR4 enhancer sequences in | |
| | the reverse orientation, 3′ to the tag sequence EGFP | |
| | (underlined). | |
| 23 | a tag sequence encoding EGFP operatively linked to an IRES | 10W |
| | element and a sequence encoding puromycin resistance | |
| | (“PuroR”). The tag sequence EGFP and PuroR are underlined | |
| | and the IRES element is indicated as bold. | |
| 24 | a tag sequence encoding a neomycin resistance gene product | 10X |
| | (“NeoR”) operatively linked to an IRES element and a | |
| | sequence encoding EGFP. The tag sequence EGFP and NeoR | |
| | are underlined and the IRES element is indicated as bold. | |

For all of SEQ ID NOS: 7-14 (Table 1), the ETn pAMu sequence may be operatively linked 5′ to a tag sequence (e.g. EGFP, NeoR-IRES-EGFP (“NIE”) or EGFP-IRES-PuroR (“EiP”), or another tag sequence as described herein). For SEQ ID NOS: 15-22, the EGFP tag sequence may be substituted by another tag sequence, e.g. NeoR-IRES-EGFP (“NIE”) or EGFP-IRES-PuroR (“EiP”) or another tag sequence as described herein.

SEQ ID NOS: 7-14 may be operatively linked 5′ to a tag sequence. Examples of tag sequences include a sequence encoding EGFP, sequences encoding a gene product for puromycin resistance, a sequence encoding a gene product for neomycin or G418 resistance, or a sequence encoding EGFP operatively linked to a sequence encoding a gene product for puromycin resistance and further comprising an operatively-linked IRES (SEQ ID NO: 23), or a sequence encoding a gene product for neomycin resistance operatively linked to a sequence encoding EGFP and further comprising an operatively linked IRES (SEQ ID NO: 24).

### Materials and Methods

Plasmid Vector Constructions

The HSC-1 retrovirus (Osborne et al., J. Virol., 1999) and PL (self-inactivating) lentivirus vector backbones (Buzina et al, 2008 PLOS Genetics in press) have been previously described. The mouse PGK promoter was derived from SM-2 vector, ETnII LTR#6 promoter is described previously (Maksakova et al., 2005) but introduced a single nucleotide mutation in poly A signal by 2 step PCR method using primers ETn-pA-Mu-s, ETn-pA-Mu-a, RVP3(Promega) and GLP2(Promega) (Table 2). Human Nanog promoter was PCR amplified from BAC RP11-277J24 (AC006517) containing human chromosome 12 using following primers: Nanog-NcoI and Nanog-BamHI. Mouse Oct-4 promoter was derived from the 2.7 kb HindIII fragment of GOF-18 GFP (Yeom et al., 1996). Mouse Oct-4 enhancer CR4 (Okumura-Nakanishi, supra) and Sox enhancer SRR2 (Tomioka, supra) were PCR amplified from genomic DNA of J1 ES cells (strain 129S4/Jae) using primers mOct4-CR4-s(EcoRI), mOct4-CR4-a(XhoI), mSox2-SRR2-s(EcoRI) and mSox2-SRR2-a(XhoI) (Table 2).

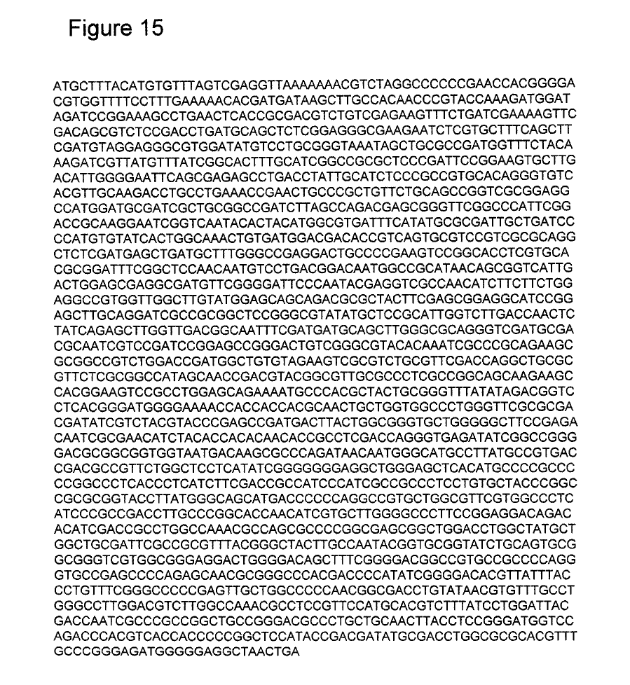

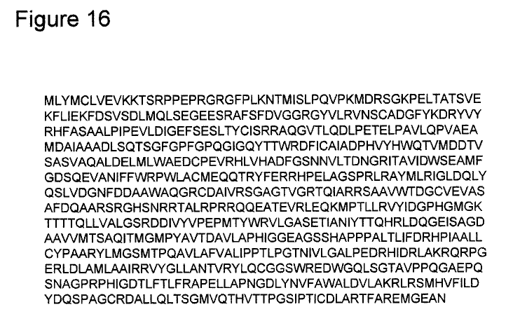

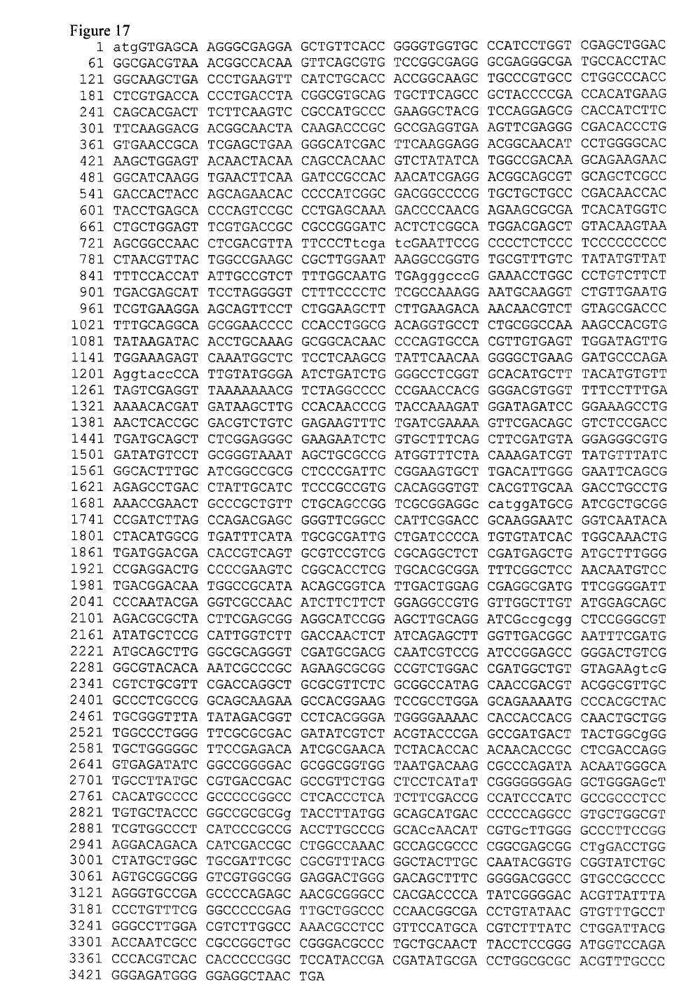

TABLE 2
Primers for amplification of promoters and enhancers,
and for genomic PCR.

| SEQ ID | | |
|---|---|---|
| NO: | Sequence | Name |
| 25 | TAGTGTCGCAACtATAAAATTTGAGC | ETn-pA-Mu-s |
| 26 | GCTCAAATTTTATaGTTGCGACACTA | ETn-pA-Mu-a |
| 27 | CTAGCAAATAGGCTGTCCC | RVP3(Promega) |
| 28 | CTTTATGTTTTTGGCGTCTTCC | GLP2(Promega) |
| 29 | gcCCATGGTGTTAGTATAGAGGAAGAGG | Nanog-Nco1 |
| 30 | taGGATCCAAAAGTCAGCTTGTGTGG | Nanog-BamH1 |
| 31 | ggaGAATTCGGGTGTGGGGAGGTTGTA | mOct4-CR4-s(EcoRI) |
| 32 | aagCTCGAGCTAGGACGAGAGGGACCCCT | mOct4-CR4-a(XhoI) |
| 33 | attGAATTCCCAGTCCAAGCTAGGCAGGT | mSox2-SRR2-s(EcoRI) |
| 34 | ctaCTCGAGAGCAAGAACTGTCGACTGTGCT | mSox2-SRR2-a(XhoI) |
| 35 | AACGGGGTAGAAAGCCTG | IMR3912 (common forward primer) |
| 36 | TGATGGGGTCCTCAGAGC | IMR3913 (WT allele specific reverse primer) |
| 37 | ATGCTCCAGACTGCCTTG | IMR3914 (MUT allele specific reverse primer) |
| 38 | CGCTCTGCCCTATCT CTGAC | RTT-Fwd |
| 39 | AGTCCTTTCCCGCTCTTCTC | RTT-Rev |
| 40 | (see FIG. 15) | HygroTK nucleic acid |
| 41 | (see FIG. 16) | HygroTK amino acid |
| 42 | (see FIG. 17) | EGFP-IRES-HygroTK construct |

All promoters and enhancers were confirmed by DNA sequencing.

Cell Culture

J1 mouse ES cells were cultured on gelatin-coated dishes using mouse ES medium (DMEM with 15% FBS supplement with 4 mM L-glutamin, 0.1 mM MEM non-essential amino acids, 1 mM sodium pyruvate, 0.55 mM 2-mercaptoethanol, and LIF), unless specified. Plat-E cells (Morita et al., 2000) were maintained in DMEM with 10% FBS containing blasticidin (10 μg/ml) and puromycin (1 μg/ml). 293T, NIH3T3 and MEF cells were cultured in DMEM with 10% FBS supplement with 4 mM L-glutamine. MEFs were isolated from E15.5-E17.5 CD-1 mouse embryos.

Human ES cell line CA1 was maintained on feeders in Knockout DMEM (Invitrogen) supplemented with 15% Serum Replacement (Invitrogen), 2 mM Glutamax (Invitrogen), penicillin/streptomycin, 0.1 mM non-essential amino acids, 0.5 mM mercaptoethanol, and 10 ng/mL recombinant FGF2 (Peprotech). Human embryonic stem cells were grown on matrigel in the presence of MEF-conditioned medium as previously reported (Bendall et al., 2007). Human dermal fibroblasts (HDFs) were isolated skin biopsy from 8-years old male by distal humerus osteotomy. Feeder cells for CA-1 and iPS cultures were isolated from E15.5 embryo of Tg(DR4)1Jae/J mice (Stock No. 003208, Jackson Laboratory) for puromycin resistance.

Virus Production and Infection

Production of retroviral and lentiviral vectors were as described previously (Buzina et al., 2008; Hotta et al., 2006). Briefly, Plat-E cells were plated at a density of 1×10⁵ cells/cm². Next day, the cells were transfected with the appropriate plasmids using 1 μl/1×10⁵ cells of Lipofectamine 2000 (Invitrogen).

For lentiviral EOS vector production, 293T cells were plated at a density of 8×10⁶ in T-75 flasks. The following day, the cells were transfected using Lipofectamine 2000 (Invitrogen) with 10 μg HPV275 (gag/pol expression plasmid), 10 μg P633 (rev expression plasmid), 10 μg HPV17 (tat expression plasmid), 5 μg pVSV-G (VSV-G expression plasmid) and 15 μg of EOS lentiviral plasmid which is derived from the PL.SIN.EF1a-EGFP backbone (Buzina et al, 2008). The lentiviruses were collected in 20 mL media after 48 hours, filtered through 0.45 μM filters to remove cell debris. If necessary, viruses were concentrated by ultracentrifugation at 4° C., 2 hours, 30,000 rpm with T-865 rotor (Sorvall). The viral pellet was resuspended in 40 μl Hanks' balanced salt solution (Invitrogen) overnight at 4° C. Titer for PL-EOS-C(3+)-EiP lentiviral vectors was approximately 1×10⁷ IU/ml assayed on J1 mouse ES cells, and the titer was used to estimate the MOI of fibroblast infections.

One day before infection, target cells were seeded at 5×10⁴ cells (for NIH3T3 and MEFs) or 1×10⁴ cells (for J1) per wells of a 24-well plate. For infection, virus was added to the target cells with several dilutions in the presence of 8 microgram/ml polybrene (hexadimethrine bromide, Sigma). Twenty four hours post infection, virus was removed and transgene expression was analyzed 2 to 3 days post infection.

Surface Marker Staining

Cells were trypsinized into single cell suspension and incubated with Mouse IgM anti SSEA-1 antibody (MC-480, Hybridoma Bank) for 30 min on ice. After washing with PBS, cells were incubated with PE-Cy5.5 conjugated anti mouse IgM antibody (35-5790, eBioscience) for 30 min on ice.

Flow Cytometry

Trypsinized cells were suspended in PBS with 5% FBS. Single cell suspensions were filtered through 70 μm pore nylon membrane and analyzed by a FACScan (Becton Dickinson) flow cytometry using CellQuest software. Before each experiment, the machine was calibrated using calibration beads (FL-2056-2, Spherotech). Cell debris was excluded from analysis by using forward- and side-scatter gating. In each cell type, mock-infected or non-infected cells were used as a negative control to adjust FL1 gain to detect EGFP fluorescence. Obtained data were analyzed by FlowJo software (Tree Star Inc.).

Immunocytochemistry

Cells were fixed with 4% formaldehyde in PBS for 20 min, permeabilized with 0.2% NP-40 for 5 min, blocked with 0.5% BSA and 6% normal goat serum for 1-2 hours, and incubated with primary antibodies with 0.25% BSA and 3% normal goat serum in PBS overnight. After washing 3 times with PBS, cells were incubated with secondary antibodies for 45 minutes. Immunostaining images were taken with a Zeiss Axiovert 200M microscope equipped with AxioCam HRm camera and AxioVision software. Antibodies used in this study are listed in Table 3.

Microscopy Imaging

Live cell images were captured using a Leica DM IL inverted contrasting microscope equipped with Leica DC500 digital color camera by OpenLab software. Acquired images are copied onto Microsoft PowerPoint software and phase-contrast images were converted to gray scale. For EGFP fluorescence, band-pass 450-490 nm filter was used for excitation and low-pass 520 nm filter was used for detection of fluorescence.

TABLE 3
antibodies used in cell staining, flow cytometry and imaging experiments

| Antibody | Catalog # | Supplier |
|---|---|---|
| Alexa 647 conjugated anti rat IgM | A21248 | Invitrogen |
| PE-Cy5.5 conjugated anti mouse | 35-5790 | eBioscience |
| IgM | | |
| Cy3 conjugated anti mouse IgG | 715-165-151 | Jackson ImmunoResearch |
| Cy3 conjugated anti mouse IgM | 115-165-020 | Jackson ImmunoResearch |
| Rhodamine conjugated anti rabbit | 111-0250144 | Jackson ImmunoResearch |
| Mouse IgM anti TRA-1-81 | 41-1100 | Invitrogen |
| Mouse IgM anti TRA-1-60 | 41-1000 | Invitrogen |
| Rat IgM anti SSEA-3 | MC631 | Developmental Studies Hybridoma |
| | | Bank |
| Mouse IgG anti SSEA-4 | MC813-70 | Developmental Studies Hybridoma |
| | | Bank |
| Mouse IgG anti anti-smooth muscle | M-7786 | Sigma |
| actin | | |
| Rabbit IgG anti GATA4 | SC-9053 | Santa Cruz |
| Rabbit IgG anti Nestin | MAB5922 | Chemicon |
| Mouse IgG anti alpha-actinin | sc-59953 | Santa Cruz |
| Mouse IgG anti alpha-Fetoprotein | MAB1368 | R&D Systems |
| Mouse IgG anti beta-III tubulin | MAB1637 | Chemicon |
| Rabbit polyclonal anti Nanog | RECRCAB0002PF | CosmoBio |
| Mouse IgM anti SSEA-1 | MC-480 | Developmental Studies Hybridoma |
| | | Bank |

Mouse ES Cell Differentiation

J1 ES cell colonies cultured on gelatin-coated dishes were loosely detached by trypsin-EDTA treatment and suspended in mouse ES medium without LIF. The J1 ES colonies were cultured as suspension in non-treated Petri dishes for 4 days to make embryoid bodies (EB). The cells were treated with 5 μM all trans retinoic acid (RA, Sigma) for 24 hours and cultured further as EBs for 3 days. The EBs were trypsinized to suspend into single cells and plated onto tissue culture grade dishes for an additional 3-5 days.

Alkaline Phosphatase Staining

Cells were fixed by 4% formaldehyde and stained by 1 mg/ml Fast Red TR hemi (zinc chloride) salt (F8764, Sigma) and 0.4 mg/ml Naphthol phosphate disodium salt (N7255, Sigma) in 0.1M Tris-HCl (pH=8.6) for 10 min at room temperature. Wild type J1 ES cells were used for staining control and NIH3T3 or MEF (mouse embryonic fibroblast) cells were used for negative control.

Mouse iPS Cell Induction

The induction of iPS cells was performed based on the Yamanaka protocol (Nakagawa et al., Nature Biotechnology, 2007; Takahashi et al., Nature Protocols, 2007). In brief, retrovirus vectors encoding Oct-4, Sox2, Klf4, and c-Myc were produced using Plat-E cells by plasmid transfection of either pMXs-Oct4, pMXs-Sox2, pMXs-K1f4, or pMXs-c-Myc (Addgene plasmid 13366, 13367, 13370, and 13375, respectively). One million cells per 10 cm dish of MEFs (isolated from wild type strain CD-1 or MeCP2 mutant mice [Stock No. 005439 Jackson Laboratory]) were infected with 2.5 ml each of unconcentrated retrovirus vector in the presence of 8 μg/ml polybrene. One day after infection, the cells were trypsinized and 6×10⁵ cells were transferred onto feeder cells in a 10 cm dish in mouse ES media. Colonies were picked and dissociated by trypsinization. All EOS infected iPS cell lines were maintained in mouse ES media containing 1 μg/ml puromycin on feeders.

Human iPS Cell Induction

Human BJ fibroblasts (ATCC, CRL-2522) or Rett Syndrome patient fibroblasts (Coriell, GM11270) were infected with pLenti6/UbC/mS1c7a1 lentiviral vector (Addgene, 17224) expressing the mouse S1c7a1 gene and selected with blasticidin prior to reprogramming experiments. Cells were seeded at 8×10⁵ cells per 10 cm dish and transduced twice with pMXs retroviral vectors encoding hOCT4, hSOX2, hKLF4, and hc-MYC (Addgene 17217, 17218, 17219, and 17220, respectively) (Lowry et al., 2008), together with pMXs-mRFP1 (monomeric Red Fluorescence Protein 1) retrovirus for monitoring infectivity and viral silencing. One week after transduction, cells were trypsinized and seeded onto 10 cm feeder dish in human ES cell media. Emerged colonies were picked and mechanically dissociated at initial passages up to the 6-well plate, then adapted to collagenase treatment. All EOS infected iPS cell lines are maintained in human ES media containing 1 μg/ml puromycin on feeders.

Teratoma Formation

Mouse iPS cells were suspended in PBS with 5% FBS and injected into the testes of NOD/SCID mice. Four to five weeks after injection, tumors were weighed. Human iPS cells were suspended in a mixture of KO-DMEM, Matrigel and collagen to inject intramuscularly into NOD/SCID mice, as previously described (Park et al., 2008). Tumors were harvested 9 weeks after injection. Fixed tumors were embedded in paraffin, sectioned and stained with hematoxylin and eosin for pathological analysis. Mouse and human ES cells were used as positive control for teratoma formation. Parental fibroblasts for iPS derivation did not form teratomas.

Genotyping of MeCP2 Mutation

For mouse RTT-iPS cells, PCR on genomic DNA yielded an amplicon of 396 by for wild-type Mecp2 and 318 by for the truncated Mecp2³⁰⁸ allele using the following primers: (see Table 2 for sequences) IMR3912 (common forward primer) (SEQ ID NO: 35), IMR3913 (WT allele specific reverse primer) (SEQ ID NO: 36), IMR3914 (MUT allele specific reverse primer) (SEQ ID NO: 37). For human RTT-iPS cells, genomic DNA was extracted from R306C hiPS cells and PCR was performed using the following primers: RTT-Fwd (SEQ ID NO: 38) and RTT-Rev (SEQ ID NO: 39). The PCR amplicon was isolated and DNA sequencing was performed using the RTT-Fwd Primer.

### Example 1

### Infectivity and Expression Level of Gammaretroviral Vectors in ES Cells

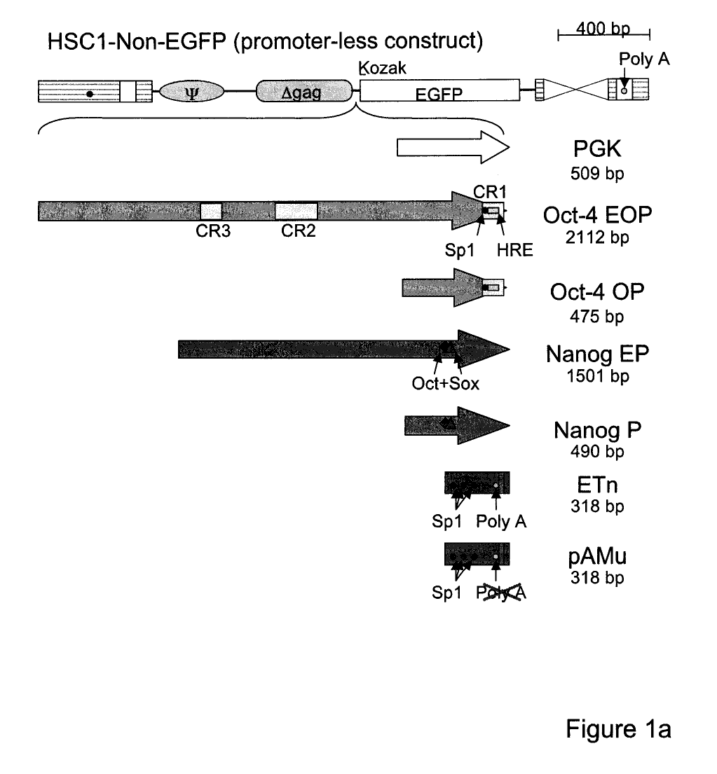

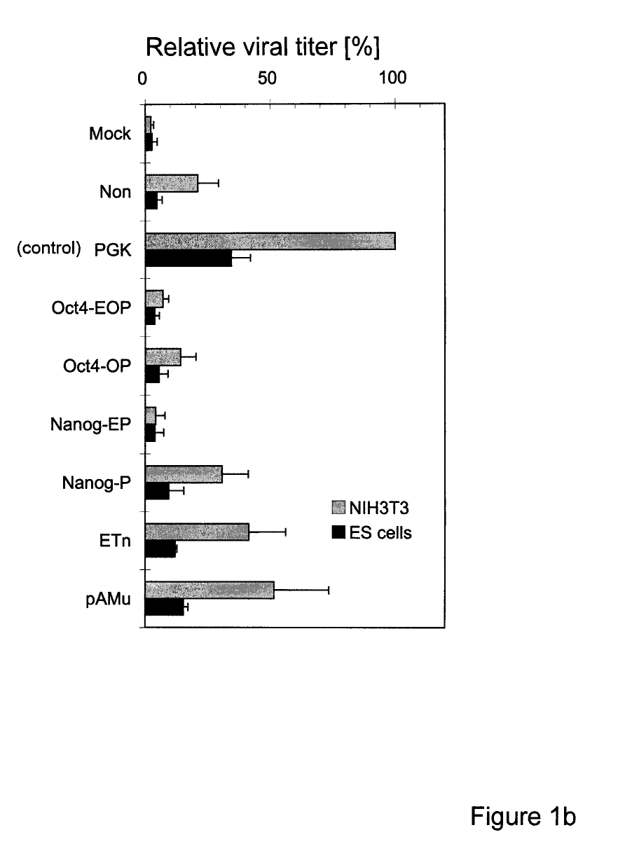

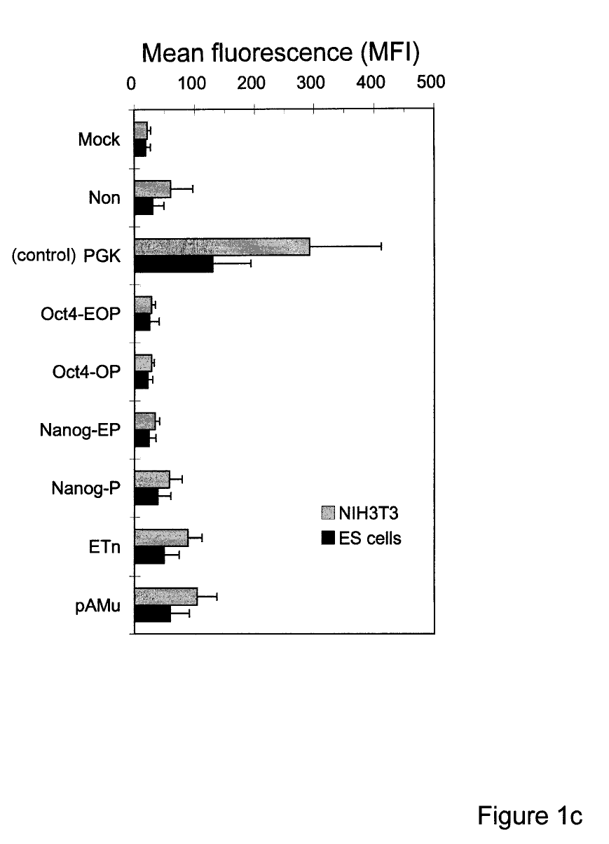

To characterize the transduction efficiency of ES cells by gammaretroviral vectors, we inserted several ES-specific or ubiquitous promoters into HSC1 vector backbone, as an internal promoter (FIG. 1a). HSC1 vector has a self-inactivating (SIN) deletion in 3′LTR U3 region and this deletion will be copied into 5′LTR upon reverse transcription. Therefore, any known silencer element binding sites are removed after integration. As SIN LTRs have negligible promoter activity, EGFP expression is solely driven from internal promoter. Produced viruses were infected simultaneously into J1 mouse ES cells and NIH3T3 mouse fibroblasts to analyze infectivity (percentage of GFP⁺ cells) and EGFP expression (mean fluorescence intensity) using flow cytometry.

### Example 2

### Nanog and Oct4 Promoters

For ES-specific expression, we tested Nanog (Nanog-EP, 1.5 kb; Nanog-P, 490 bp) and Oct4 (Oct4-EOP, 2.1 kb; Oct4-OP, 475 bp) promoters. FIGS. 1b and 1c show the relative viral liter (FIG. 1b) and mean fluorescence (FIG. 1c) of Oct4, Nanog, ETn and ETn-pAMu promoters in NIH3T3 (murine fibroblast) and murine ES (embryonic stem, or mES) cells.

Both Nanog and Oct4 promoters express to low levels in ES cells. Since Nanog and Oct4 are both transcriptional factors, ES cells may not need to express those proteins to such a high level as metabolic enzymes, like PGK. A vector without promoter (LTR promoter is self-inactivation and no internal promoter) was used as a negative control to estimate the background expression of EGFP (HSC1-Non-EGFP, referred as “Non” in FIGS. 1b, c). We observed higher background expression in NIH3T3 cells than J1 ES cells, probably due to higher infectivity of retroviruses.

Given the fact that Nanog and Oct4 are not expressed in viral producer cells (293T based Plat-E) (data not shown), those promoters may work as a transcriptional repressor of 5′LTR and may be preventing virus production. Interestingly, EGFP expression from the 5′ LTR promoter was suppressed by introduction of Nanog and Oct4 promoters in the retrovirus producer cells (FIG. 2a). A similar result was observed in lentivirus producer cells (FIG. 2b).

### Example 3

### ETn Promoter and Poly A Signal Disruption

As an alternative of Nanog and Oct4 promoter, the ETn LTR promoter was tested. The ETn is an LTR-type retrotransposon and highly transcribed in pluripotent stem cells, such as ES and EC cells. Among several subfamilies of ETn promoter, we used the type II #6 LTR promoter. Surprisingly, ETn promoter has higher titer and EGFP expression compared with Nanog and Oct4 promoters in ES cells (FIGS. 1b and c). Also surprisingly, a mutated (A183T) ETn (pAMu) demonstrated a higher titer and EGFP expression than the wild-type ETn promoter in both ES and NIH3T3 cells (FIGS. 1b and c).

### Example 4

### Core Enhancer Elements of Oct4 and Sox2

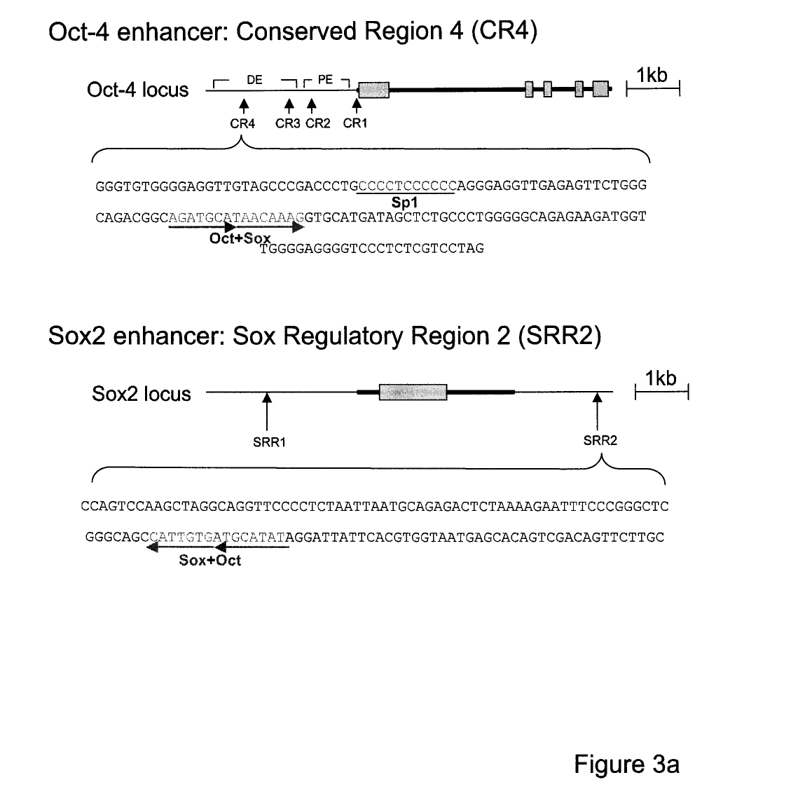

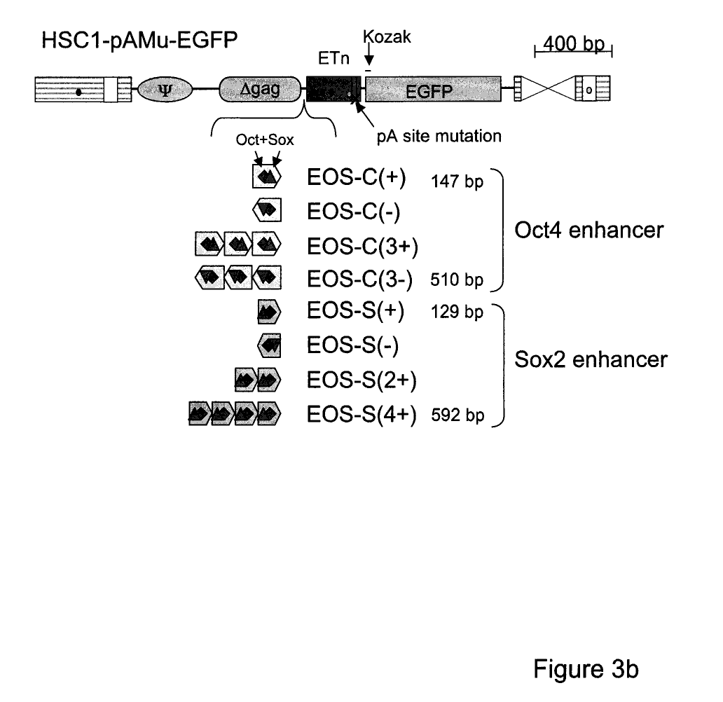

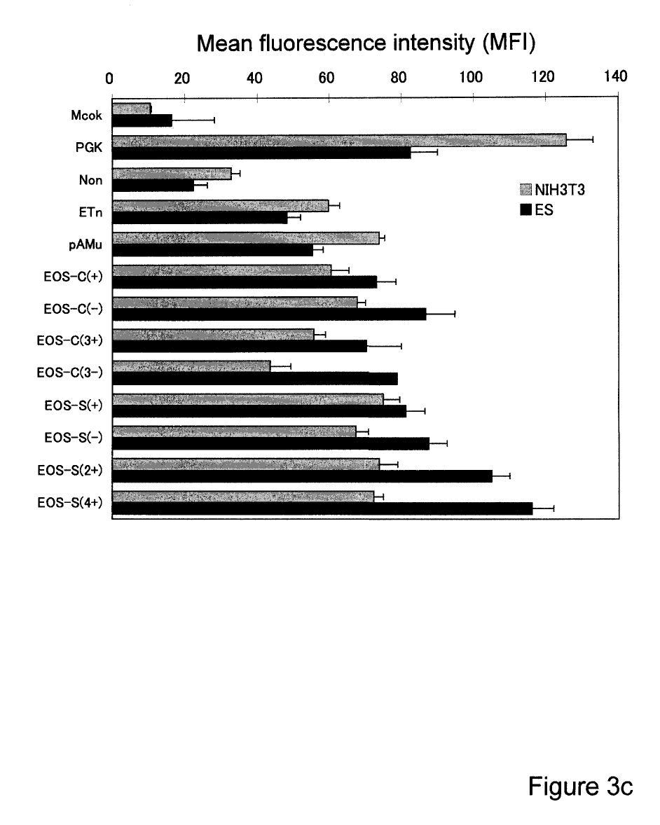

To investigate whether expression from the ETn promoter can be increased by one or more ES-specific enhancer elements, we cloned Oct4 core enhancer element (CR4) or Sox2 core enhancer element (SRR2), or a combination of CR4 and SRR2, into the HSC1-pAMu-EGFP vector (FIGS. 3a, b).

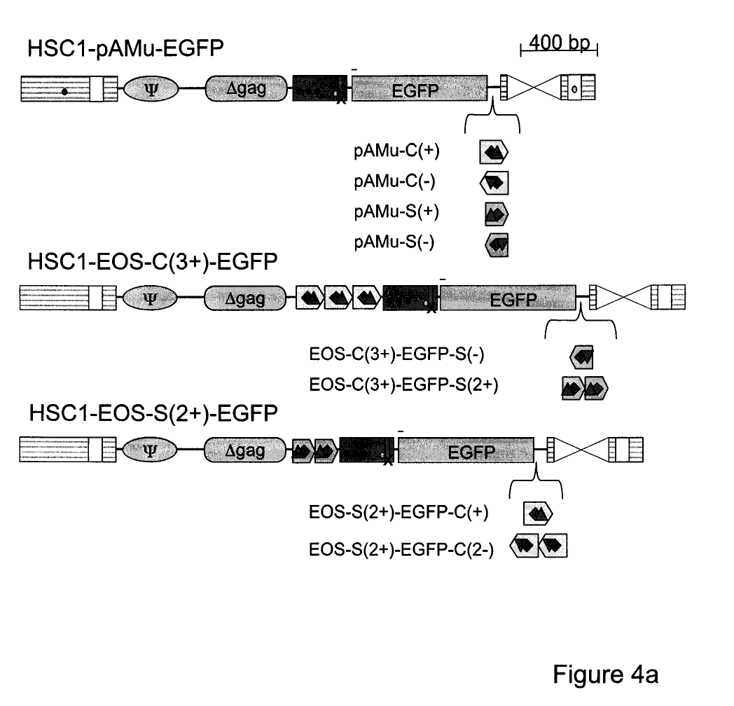

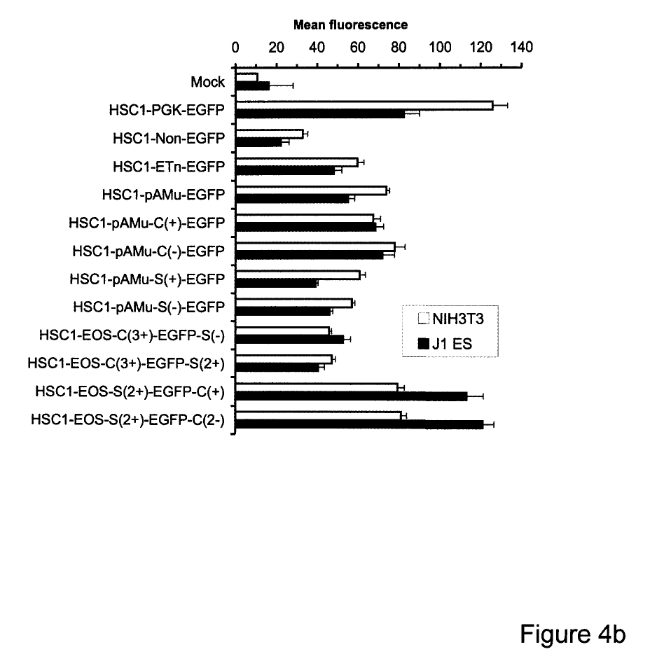

Introduction of one or more copies of CR4 (SEQ ID NO: 3 for forward orientation; SEQ ID NO: 5 for reverse orientation) or SRR2 (SEQ ID NO: 4 for forward orientation; SEQ ID NO: 6 for reverse orientation) enhancer sequences in forward or reverse orientation, or a combination thereof, upstream of the ETn pAMU promoter, EGFP expression was increased in ES cells (FIG. 3c). The resulting EOS (ETn, Oct-4, Sox2) expression cassette has ETn promoter with poly A site mutation and Oct4/Sox2 binding enhancer. The types of EOS cassette are indicated with the initial of the enhancer element (C for CR4 and S for SRR2) and copy number of the enhancer element (1 for monomer to 4 for tetramer) with direction of enhancer element(s) (“+” for forward or positive orientation, and “−” for reverse orientation). EGFP expression of those vectors in ES cells were compared to that from PGK promoter. We also introduced CR4 and SR2 enhancer elements between EGFP and the 3′LTR, in both forward and reverse orientation. The effects of enhancement were variable, depending on the construct (FIG. 4a, b).

### Example 5

### EOS Lentiviral Vectors Construction

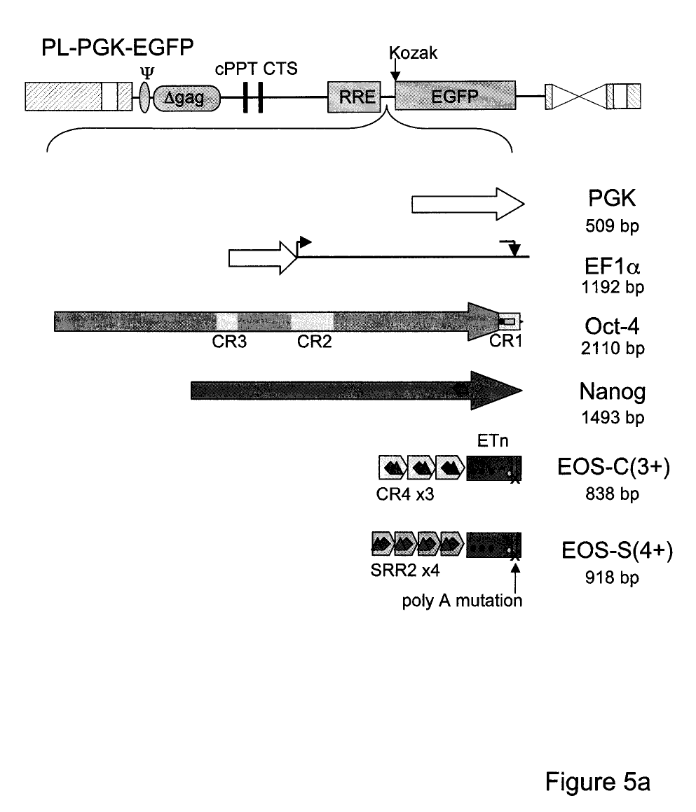

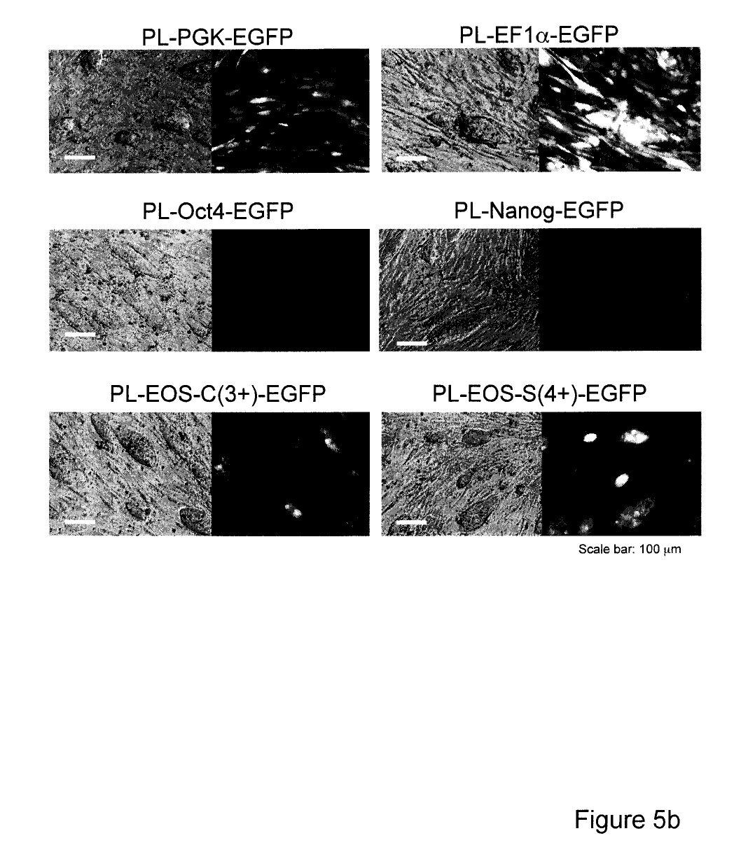

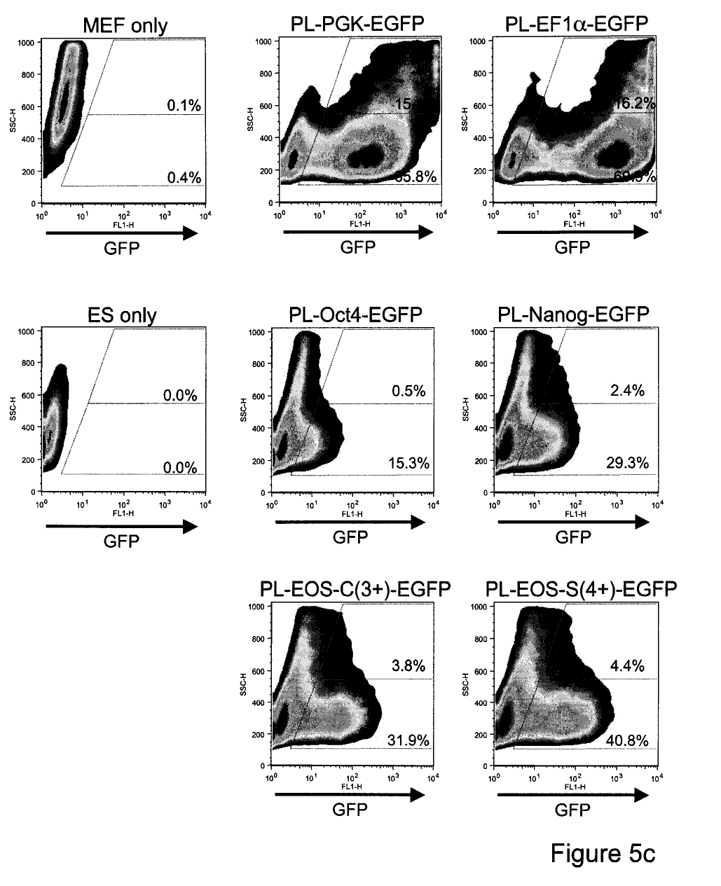

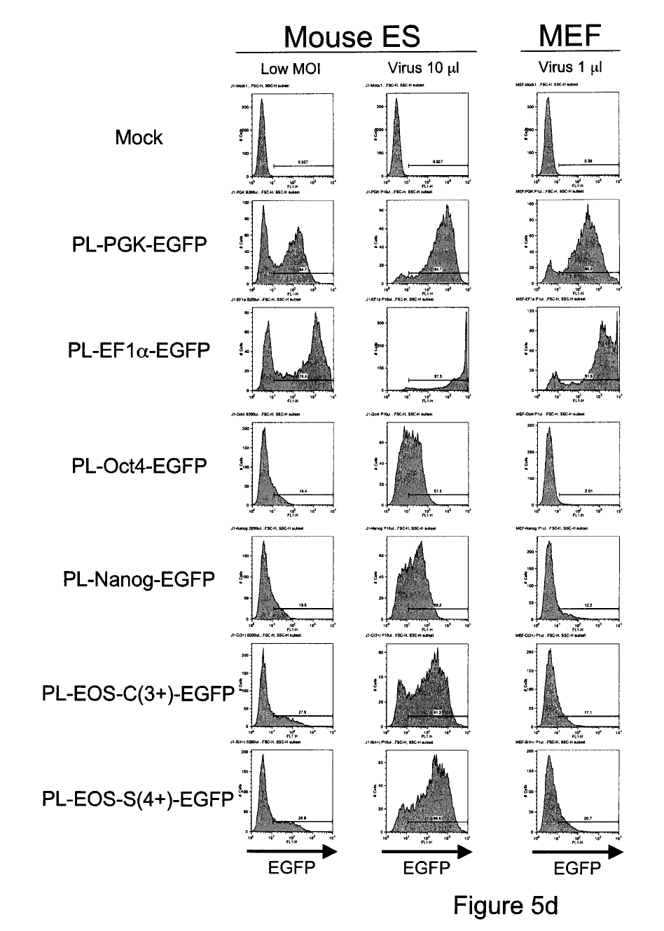

Next, to test the expression pattern of EOS cassette further, EOS constructs EOS-C(3+) and EOS-S(4+) were transferred into a self-inactivating lentiviral vector (FIG. 5a). The lentiviral vector is able to infect non-cycling cells. To show the ES-specific expression simultaneously with murine embryonic fibroblasts (MEF), we mixed mES cells and MEFs into a same well, such as ES culture on feeders. To maximize the infectivity of virus into MEFs, the feeder cells were not treated with mitomycin C, so that they still proliferated. One day after seeding of mES and MEFs, concentrated lentiviral vectors were infected and EGFP expression was analyzed by fluorescence microscopy (FIG. 5b) and flow cytometry (FIG. 5c) two days after infection. As described above, ubiquitous control vectors (EF1a, PGK) expressed EGFP higher in MEFs and to a lower level in mES cells. On the other hand, lentiviral EOS vectors [EOS-C(3+), EOS-S(4+)] have specific EGFP expression in mES cells but not in fibroblasts, and the mean fluorescence intensity (MFI) is higher than that from Oct-4 and Nanog promoters (FIG. 5d).

### Example 6

### EOS “Turn Off” after Mouse ES Cell Differentiation

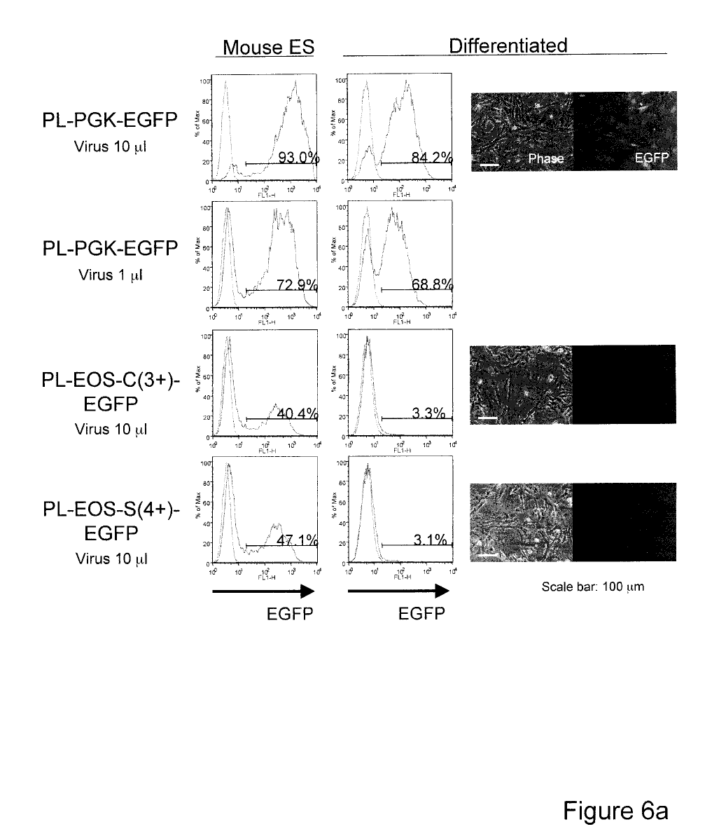

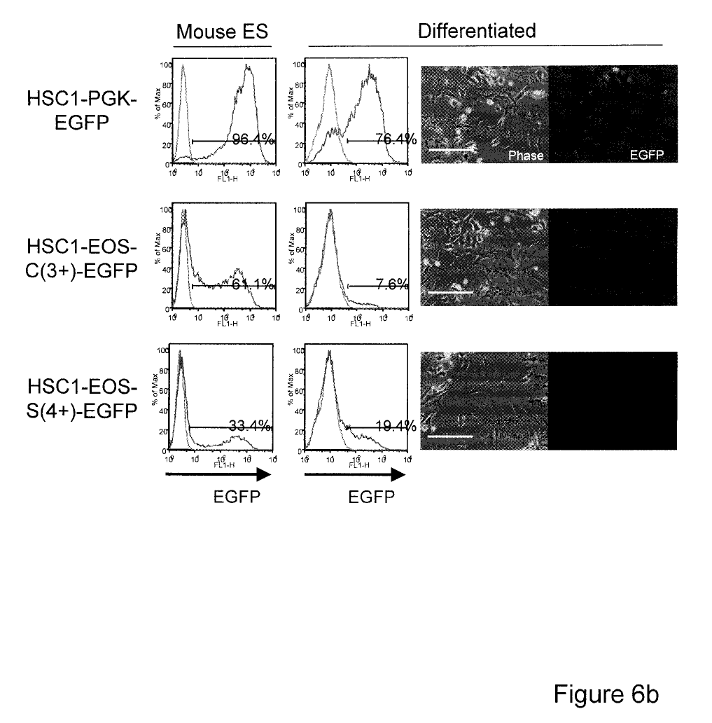

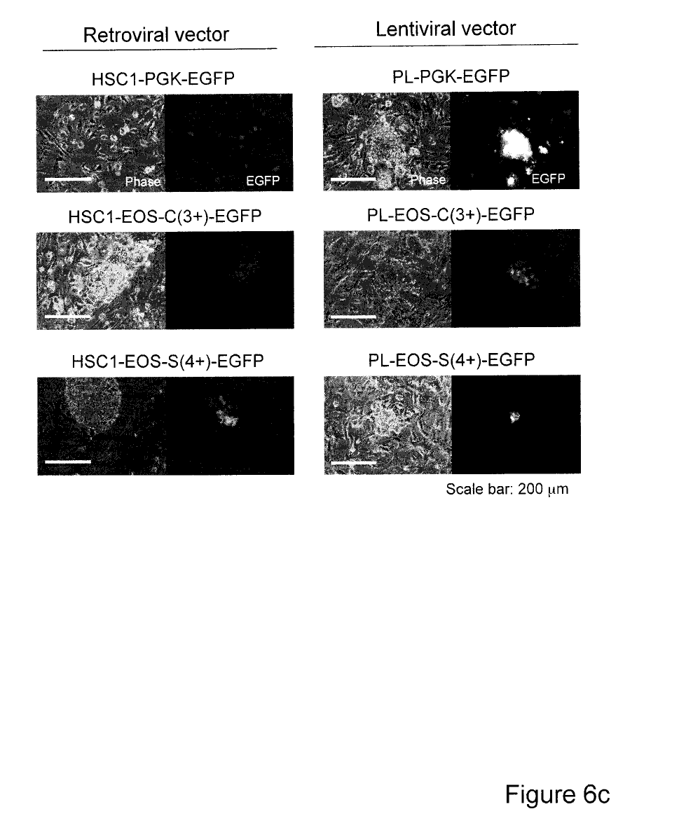

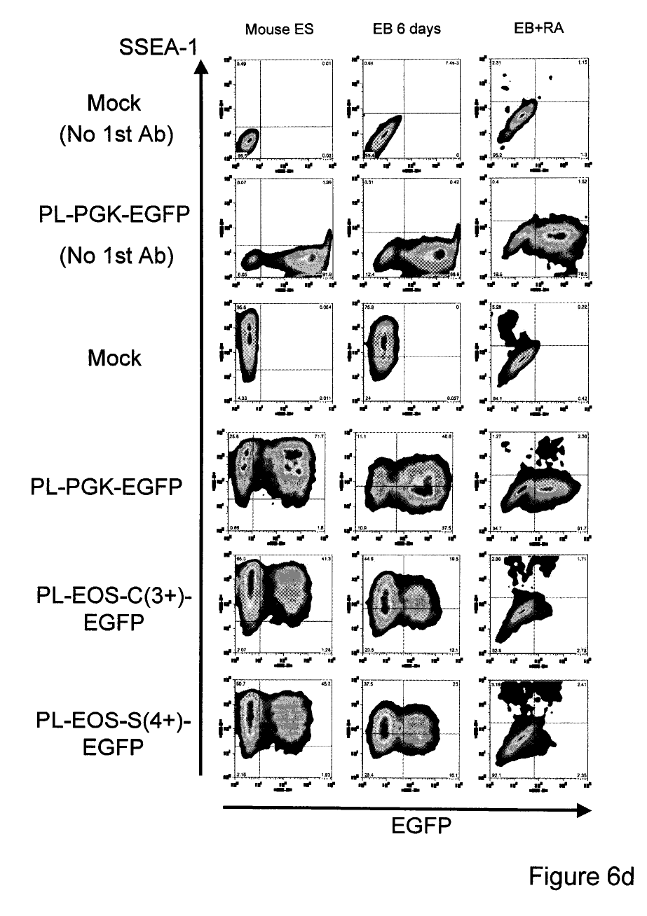

To test the specificity of the expression of the EOS cassette in the pluripotent state, we performed differentiation experiments of mouse ES cells. First, lentiviral vectors were infected into J1 cells (cultured on gelatin) and spread onto duplicate plates. One plate was maintained as an undifferentiated ES culture, and another plate was differentiated as described in the Materials and Methods. As expected, EGFP expression from the EOS cassettes diminished and was almost indistinguishable from mock-infected negative control by flow cytometry (FIG. 6a). Similar results were obtained with retroviral vector (HSC1) infected ES cells, as confirmed by flow cytometry and fluorescence microscopy (FIG. 6b).

Among several differentiation experiments, some residual GFP positive cells were observed in ES-like or EB-like colonies alter differentiation, most likely due to insufficient dissociation of EBs (FIG. 6c). These data demonstrate that EOS cassettes may be used as live-cell markers specific for undifferentiated cells.

Six day differentiated EBs express the pluripotent marker SSEA-1 (stage-specific embryonic antigen-1) in 50-75% of the cells (FIG. 6d), and even 15 day differentiated EBs still have 10-20% S SEA-1 positive cells, suggesting a heterology of EB cultures. To fully differentiate ES cells, we dissociated EBs and plated them onto a tissue culture plate. After full differentiation, the percentage of SSEA-1 positive cells was reduced to 3-5% (FIG. 6d). At the same time, the mean fluorescence of EOS cassette was diminished and almost overlaid the mock-infected negative control (FIG. 6a, d). These data demonstrate that EGFP expression by the EOS cassette is well correlated with the pluripotent cell marker S SEA-1 distribution.

### Example 7

### EOS Lentiviral Vector Expression in Human ES Cells

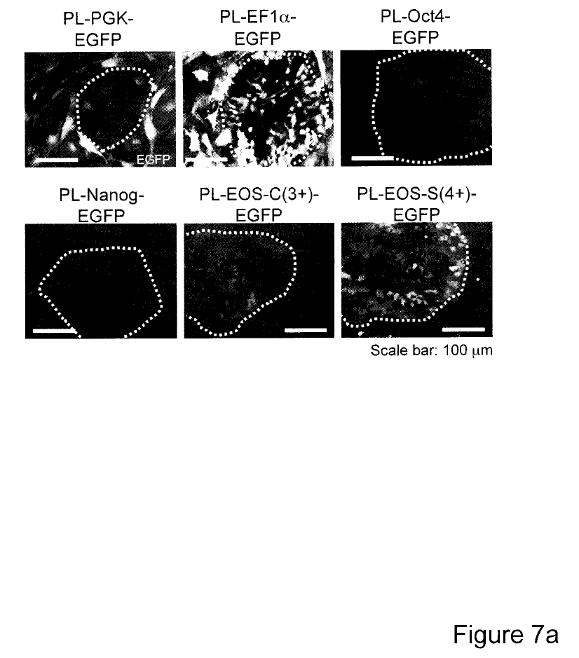

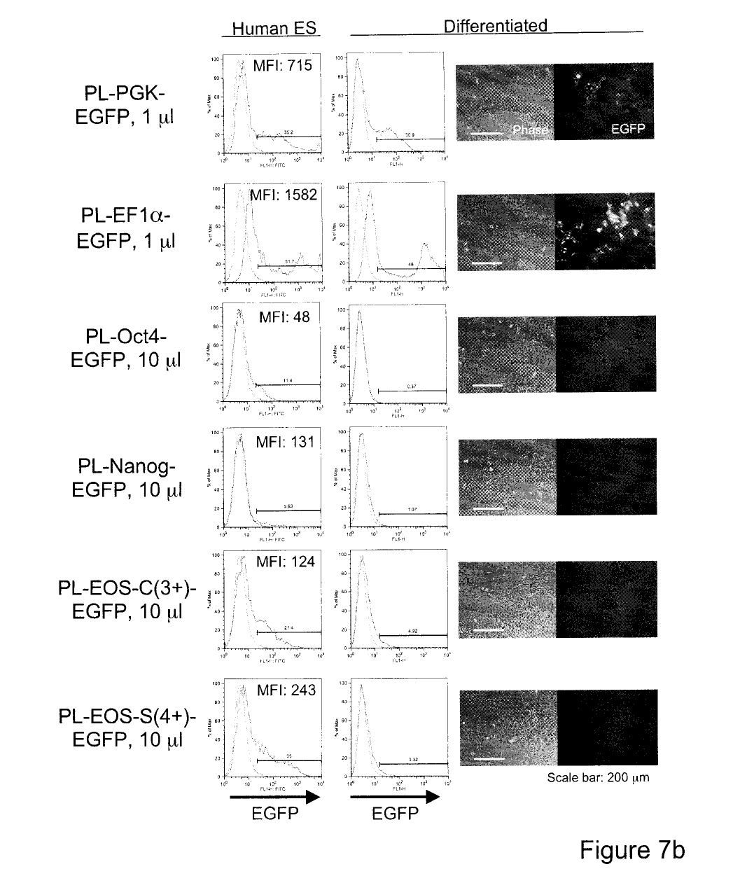

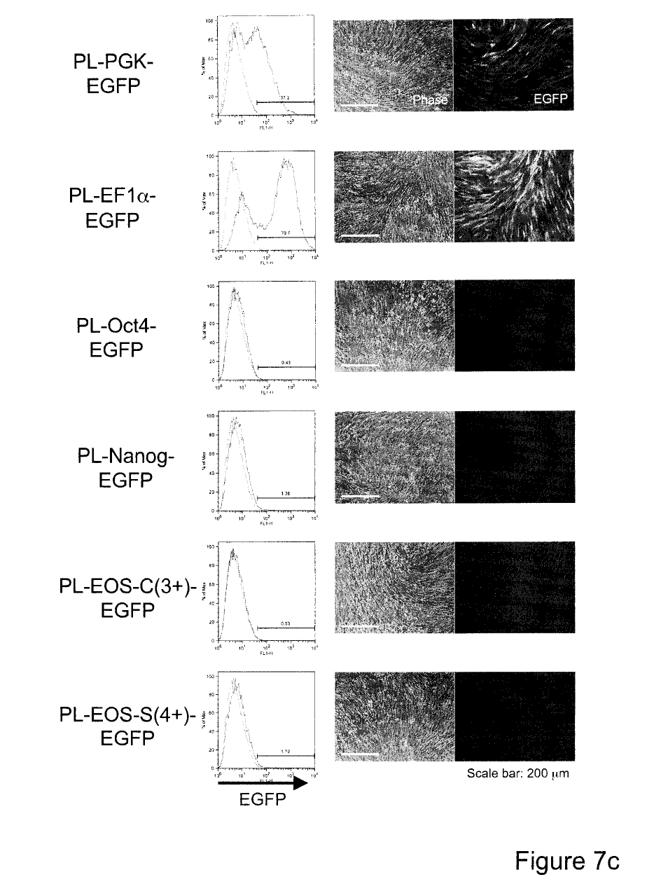

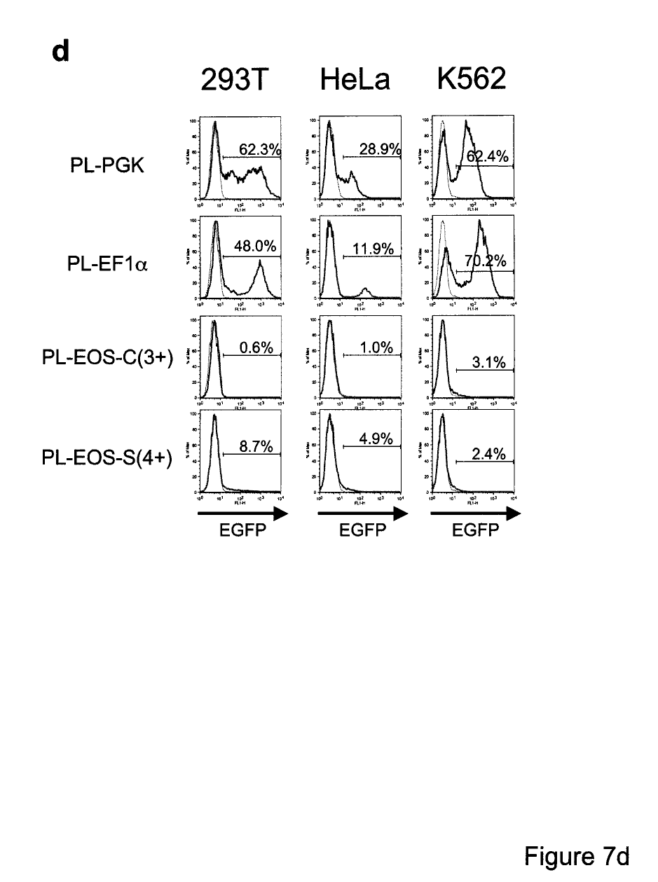

We also examined EOS expression specificity in human CA-1 ES cell lines (Peerani et al., 2007). CA-1 cells on feeders were infected with concentrated lentiviral vectors and EGFP expression from lentiviral vectors were examined 3 days after infection by fluorescence microscopy (FIG. 7a) and flow cytometry (FIG. 7b, “human ES” panels). Similar to mouse ES cell results, ubiquitous PGK and EF1a promoter have high expression in human ES cell colonies and surrounding MEF feeders. Fluorescence microscopy (3 days post infection) demonstrated specific expression of EOS lentiviral vectors in CA-1 human ES cell colonies, but not in surrounding feeder cells, whereas control PGK and EF1 alpha lentiviral vectors express in both cell types (FIG. 7a). Expression from Oct-4 and Nanog promoter vectors was difficult to detect, whereas the EOS cassettes demonstrated robust and specific expression in human ES colonies. Next, infected CA-1 human ES cells were differentiated by treatment with retinoic acid for 9 days. Three days after dissociation with trypsin-EDTA, EGFP expression was examined by fluorescence microscopy and flow cytometry (FIG. 7b). Control PGK and EF1a vectors maintained EGFP expression after differentiation, whereas ES specific promoters, Oct-4, Nanog and EOS vectors turned off after differentiation.

### Example 8

### Lentiviral EOS Vectors do not Express in Primary Human Dermal Fibroblasts

To determine whether EOS lentivirus expression is specific for pluripotent stem cells, primary human dermal fibroblasts were infected. Flow cytometry and fluorescence microscopy demonstrate that the ubiquitous PGK and Ef1α promoter vectors express in the primary fibroblasts whereas the Oct4, Nanog and EOS vectors do not (FIG. 7c). Similar infection of well-established human cell lines (293T kidney cells, HeLa epithelial cells, and K562 erythroid cells) also demonstrates that the ubiquitous PGK and EF1a promoter vectors express whereas the EOS vectors do not (FIG. 7d). These experiments suggest that EOS will be a useful marker for reprogramming primary fibroblasts or any other somatic cell type into pluripotent stem cells.

### Example 9

### Selection and Maintenance of Pluripotent Stem Cells

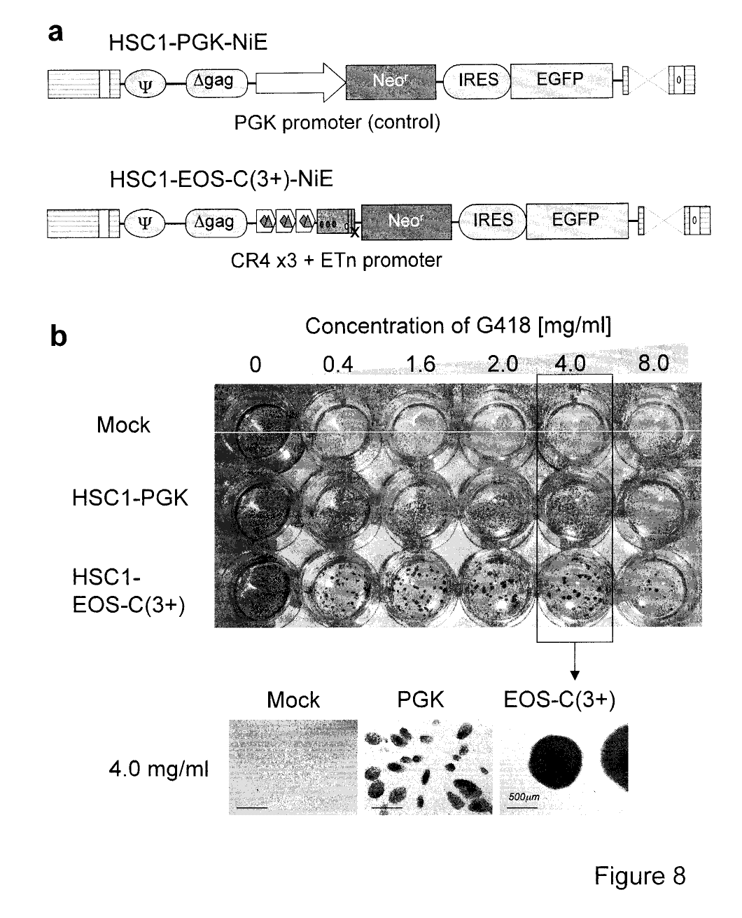

Another application of the EOS vectors is the selective growth of pluripotent stem cells expressing antibiotic resistance. A viral vector which expresses the neomycin (G418) resistance gene under the control of EOS-C(3+) promoter was constructed (FIG. 8a). J1 mouse ES cells and NIH3T3 cells were combined at a 1:5 ratio (“mixed cells”) and infected with the indicated viral vector, and 2×10⁴ mixed cells were plated into a 24-well plate and treated with different concentrations of G418 (0.4-8 mg/ml) to select expressing cells. Media was changed every 2-3 days without passage. Six days after selection, cells were fixed and stained for alkaline phosphatase activity. As shown in FIG. 8b, in the wells with low concentrations of G418 (0-0.4 mg/ml) and the control PGK vector (HPNIE), both ES cells and fibroblasts grow equally. At the same time, the edge of ES cells show weaker staining for alkaline phosphatase, indicating spontaneous differentiation because of overgrowth. On the other hand, there are big ES colonies uniformly stained for alkaline phosphatase in the wells of EOS vector infected cells (FIG. 8b). These results indicate that, under the selective pressure of EOS-C(3+) promoter expression, pluripotent stem cells can be selected from differentiated cells (such as fibroblasts), and be maintained in the pluripotent state.

### Example 10

### EOS Lentivirus Vectors Mark and Select for Reprogrammed Mouse iPS Cells

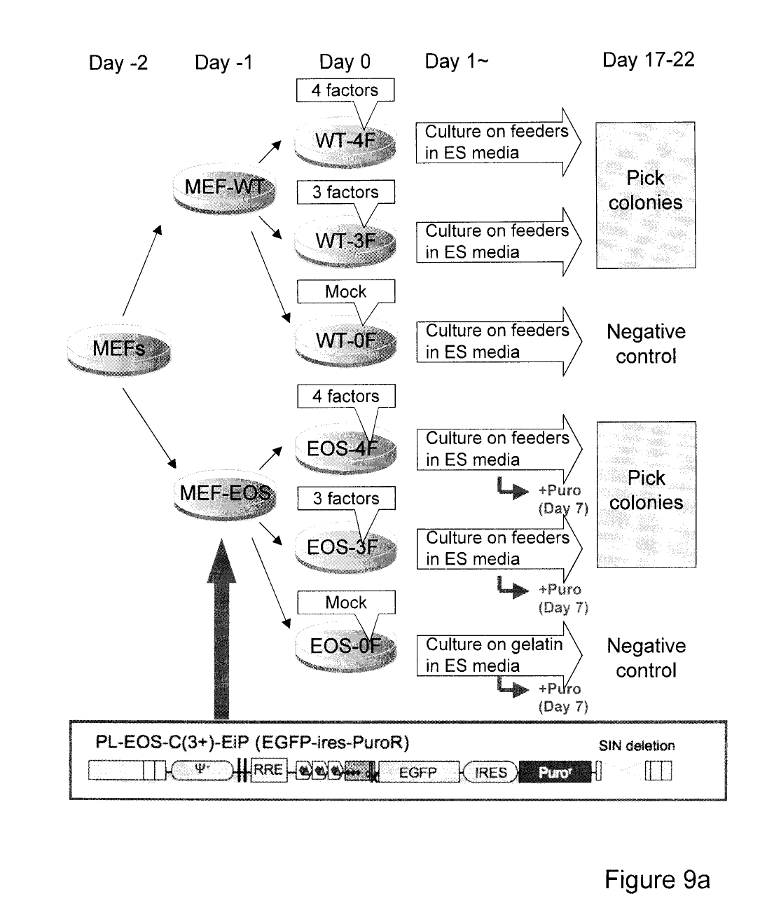

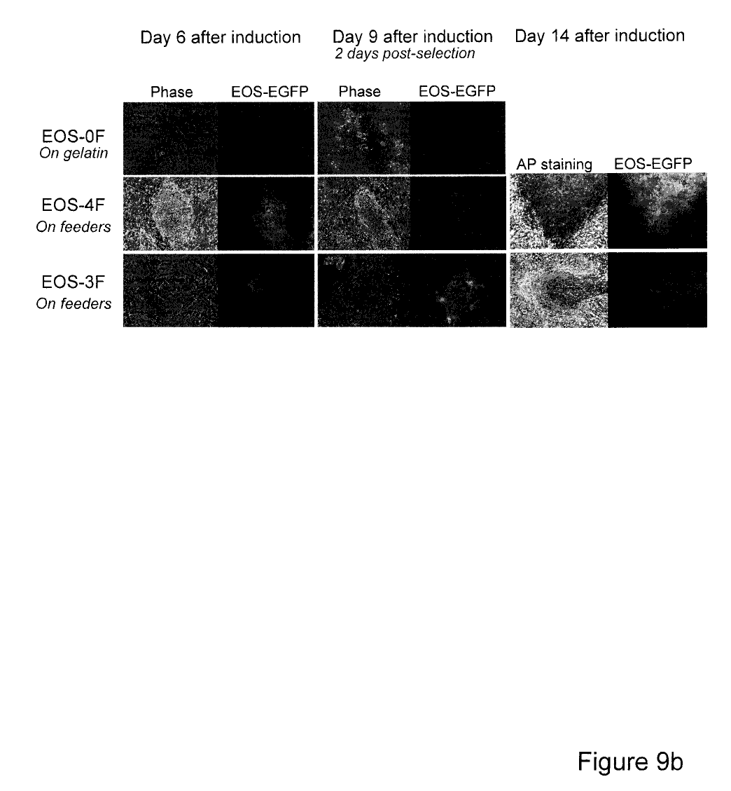

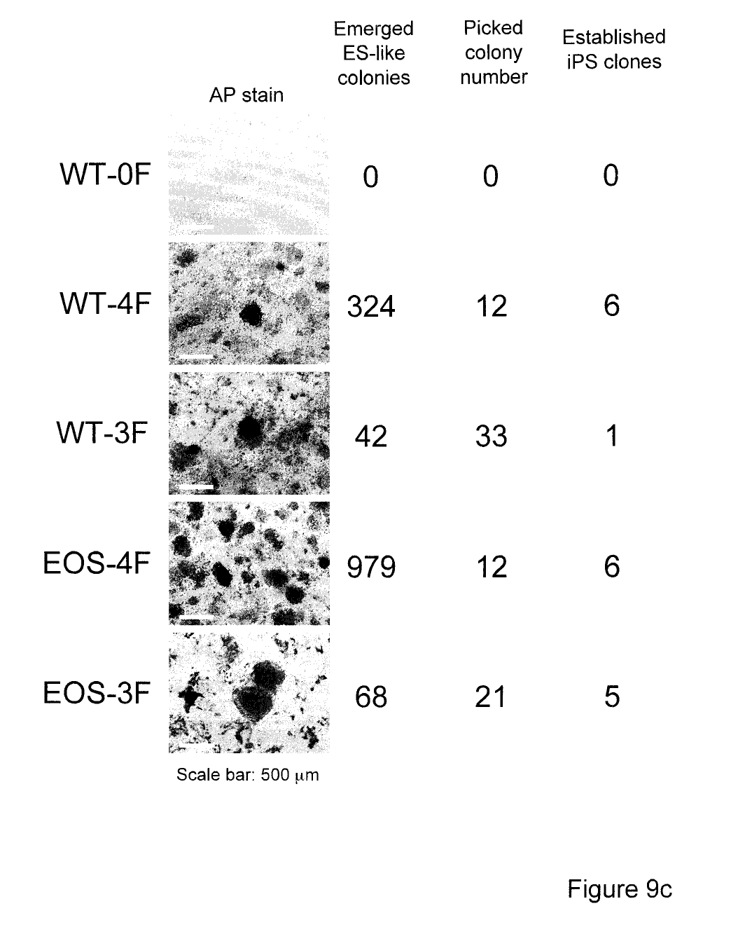

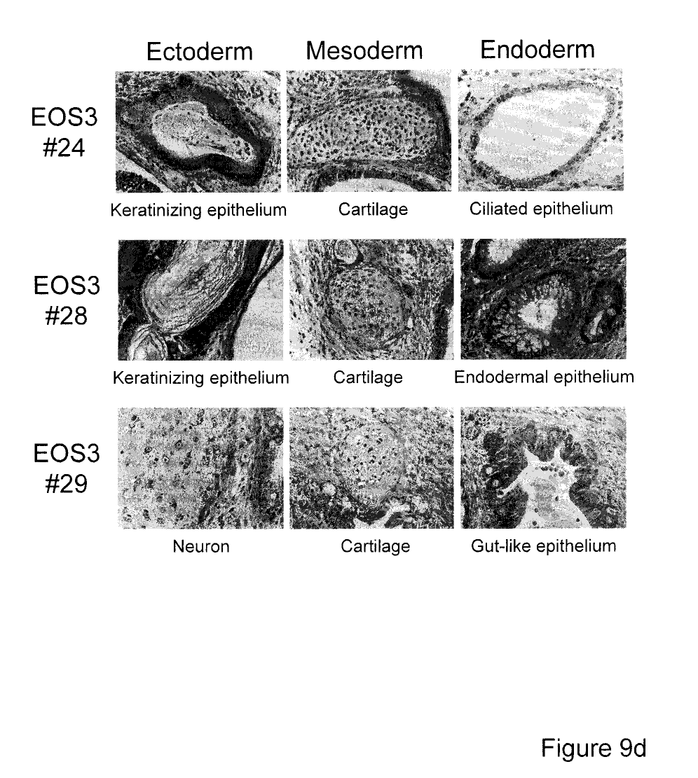

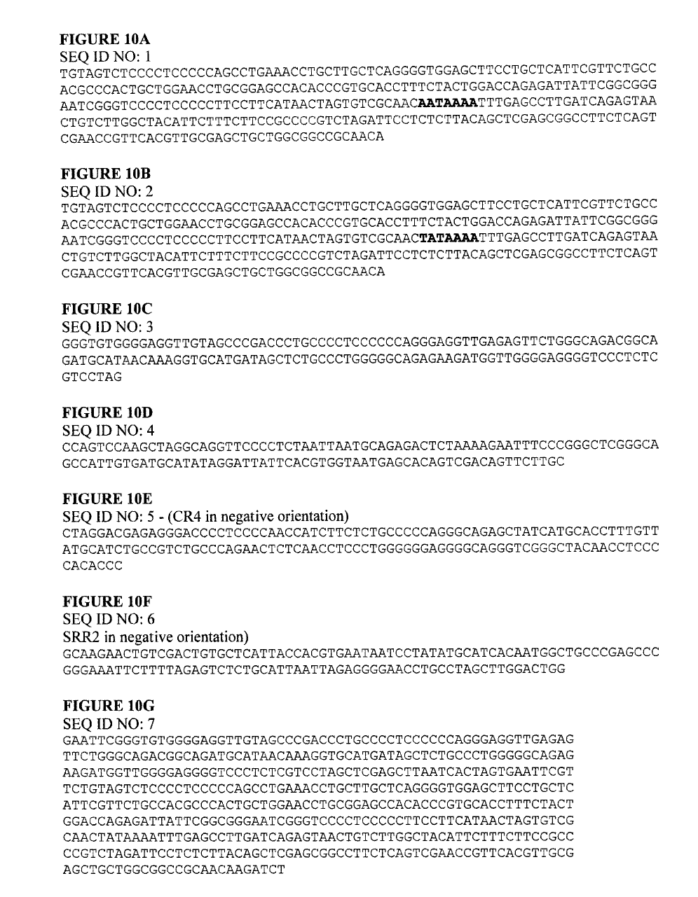

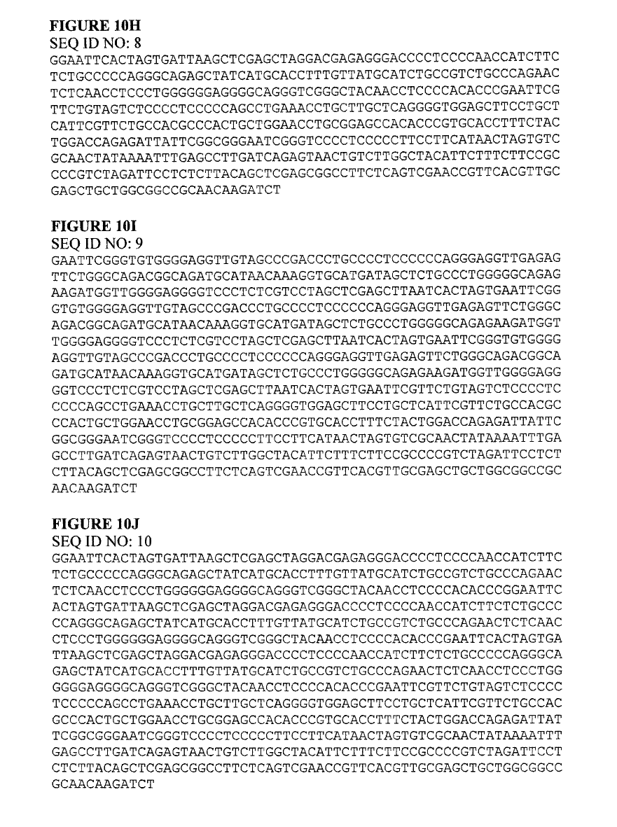

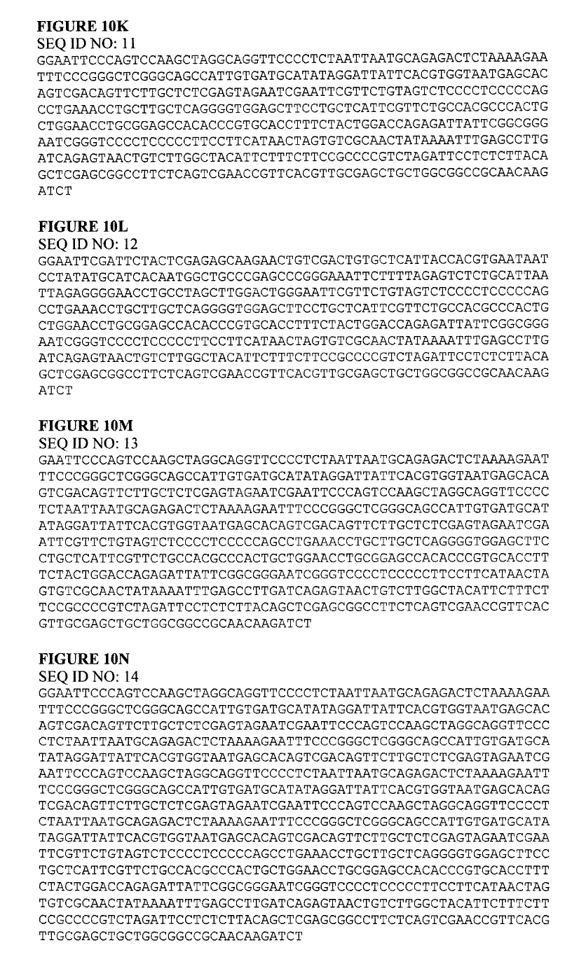

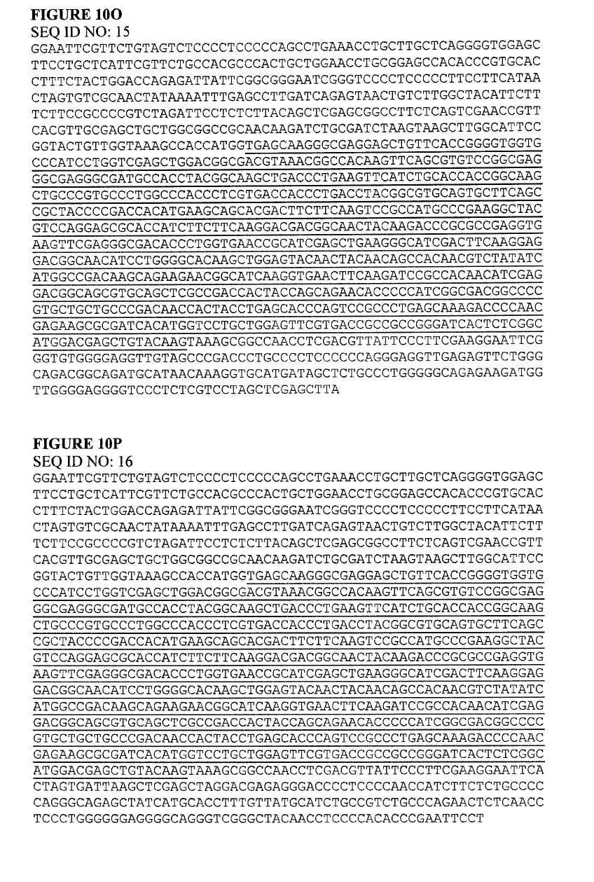

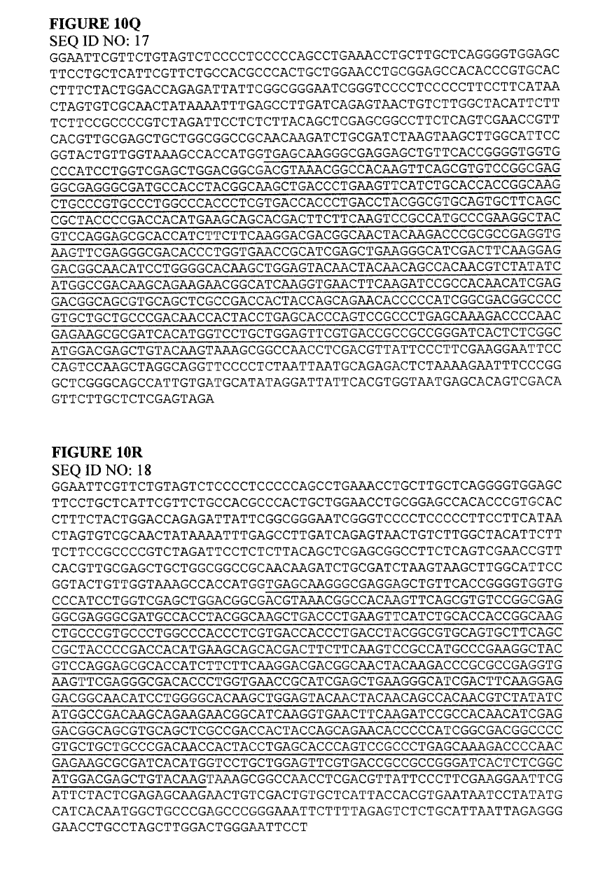

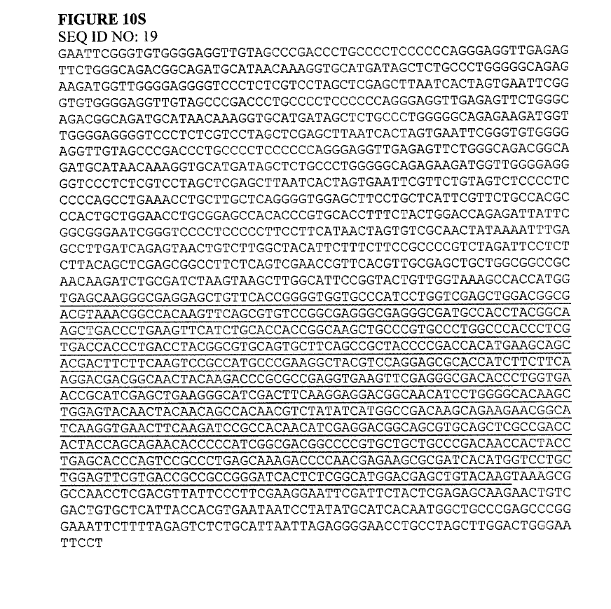

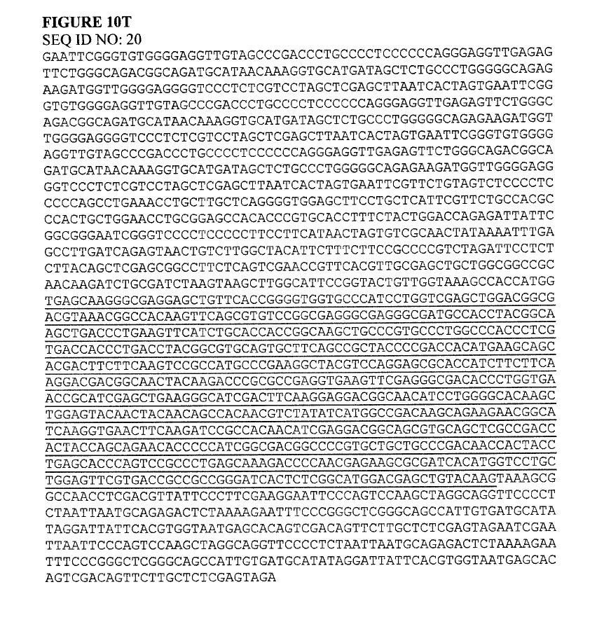

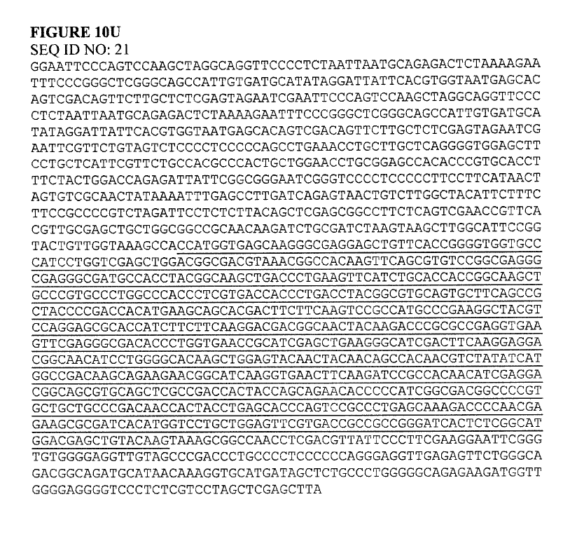

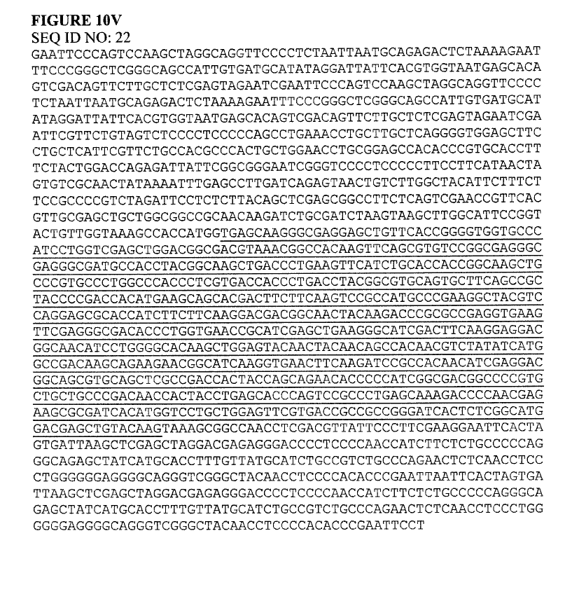

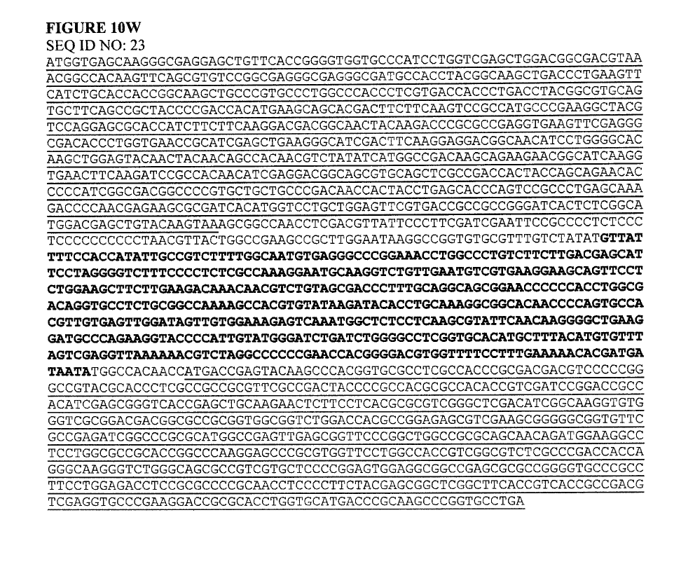

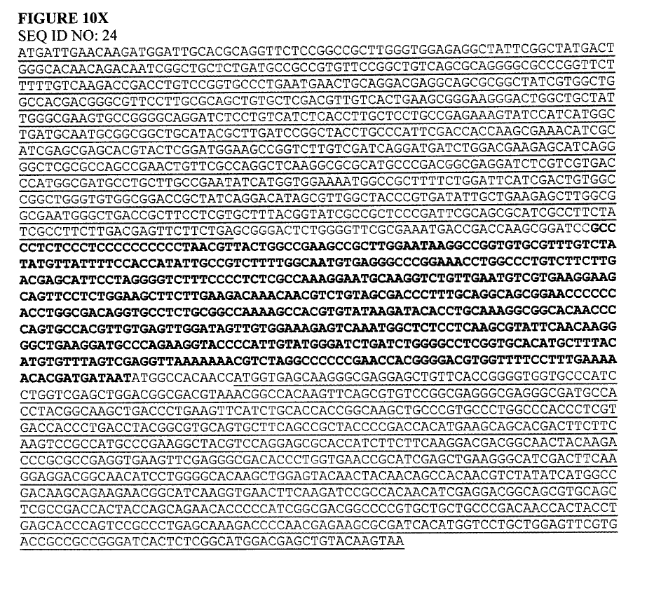

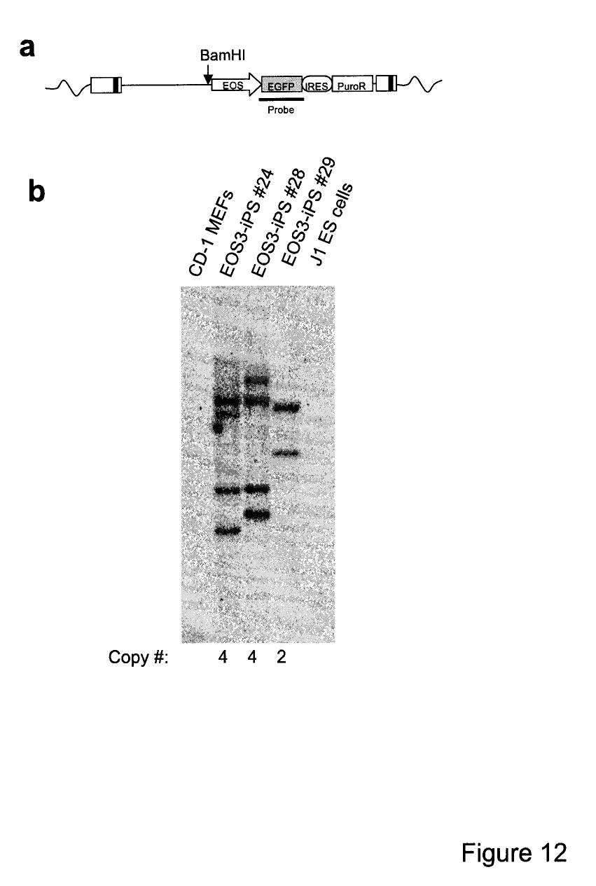

An EOS-C(3+) lentivirus vector containing an EGFP-ires-Puro cassette was constructed to mark iPS cells generated by infection of MEFs using the Yamanaka reprogramming retrovirus vectors (FIG. 9a). In brief, we attempted to reprogram WT or EOS infected MEFs (isolated from genetically unmodified CD-1 mice) by infection with retrovirus vectors encoding 4 pluripotency factors (4F: Oct-4, Sox2, Klf4, c-Myc) or 3 pluripotency factors (3F; Oct-4, Sox2, Klf4; without c-Myc). EOS-EGFP expression was monitored periodically by fluorescence microscopy and was first detected by day 6 (FIG. 9b). At day 7, the EOS vector infected MEFs were subjected to puromycin selection and survived while maintaining EGFP expression coincident with alkaline phosphatase (AP+) staining (FIG. 9b). These putative iPS colonies were picked between 17-21 days, and the remaining colonies were stained revealing an enrichment for AP positive colony numbers in the presence of EOS selection (FIG. 9c). The 4 factor inductions (EOS-4F) assumed an ES cell-like morphology in the first week, while this formation was delayed in the 3 factor inductions (EOS-3F). The 4 factor infections in the presence and absence of EOS selection (EOS-4F, WT-4F in FIG. 9c) were equally efficient at being expanded into established lines. In contrast, 3 factor inductions of WT cells (WT-3F in FIG. 9c) produced only 1 established line out of 33 colonies picked, while EOS 3 factor inductions (EOS-3 in FIG. 9c) generated 5 established lines out of 21 (FIG. 9c). The iPS cell lines show ES-like morphology and EGFP expression is coincident with the endogenous pluripotent markers S SEA-1 and Nanog. Copy number of integrated EOS lentiviral vector ranged from 2 to 4 in the lines examined by southern blot analysis (FIG. 12a, b).

These data further indicate that that EOS is an effective marker of reprogrammed colonies and pluripotency, and enriches for the isolation and maintenance of iPS cell lines.

### Example 11

### Pluripotency of Established Mouse iPS Cells

The pluripotency of established iPS clones was examined by in vitro differentiation.

After EB mediated in vitro differentiation, dissociated cells were stained for the three germ layer markers, beta-III tubulin (ectoderm), alpha-actinin (mesoderm) and alpha-fetoprotein (endoderm). Most of the lines (9 out of 10) differentiated into the three germ layers, indicating those iPS clones are pluripotent.

Three mouse iPS lines —EOS3 #24, #28 and #29—were selected for further study. To assess their in vivo pluripotency, we injected the cells into NOD/SCID mice for teratoma formation. Four to five weeks after injection, injected mice developed teratomas that contain the 3 germ layers (FIG. 9d), confirming their pluripotency in vivo.

### Example 12

### Marking Teratoma-Initiating Undifferentiated Cells after Differentiation

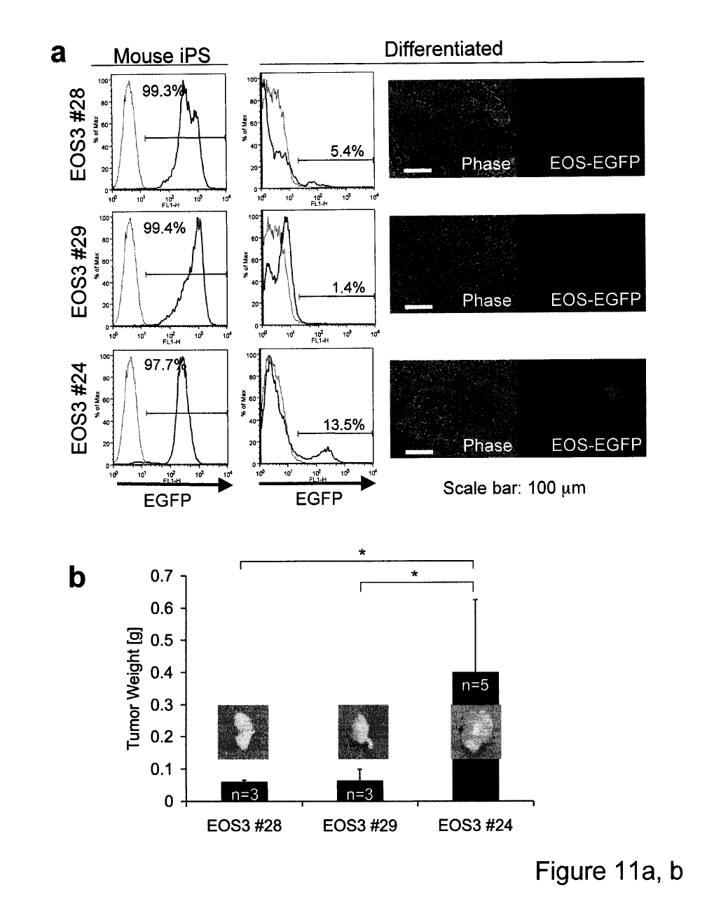

We also investigated EOS expression during differentiation of mouse iPS cell lines using the same protocol used for mouse ES cells. As expected, the EOS-EGFP expression was extinguished upon differentiation (FIG. 11a). Interestingly, one iPS cell line (EOS3#24) failed to readily differentiate but rather retain their ES-like morphology after dissociation of EBs, even after growth in normal fibroblast media without LIF and feeders. Reassuringly, these ES cell-like colonies continued to express EGFP from the EOS cassette (FIG. 11a). These results indicate that the EOS vector can be a useful live cell marker to monitor the differentiation state in vitro.

To test whether residual EOS-EGFP expression marked persisting undifferentiated cells, we injected the differentiated cells into testes of NOD/SCID mice for teratoma formation. After 5 weeks of injection, the differentiated EOS3#24 cells (EOS-GFPpositive) formed significantly larger teratomas with a wide variety of tissue types, whereas the EOS3 #28 and #29 (EOS-GFP-negative) cells had no aggressive tumorigenicity nor obvious teratoma pathology (FIG. 11). We conclude that the EOS vector is an effective live-cell marker to monitor the differentiation state in vivo, and that the EOS vector can be used to purge residual undifferentiated cells after in vitro differentiation to prevent teratoma formation by sorting EGFP negative cells, or by expressing a suicide gene (such as HSV1 thymidine kinase0.

### Example 13

### Endogenous Pluripotent Marker Expression in Human iPS Cell Lines

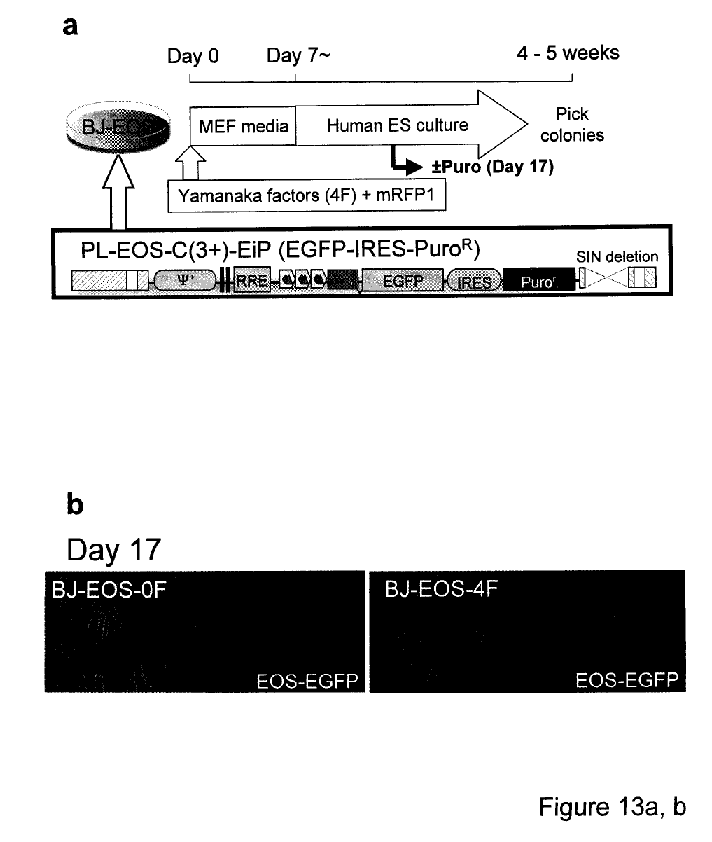

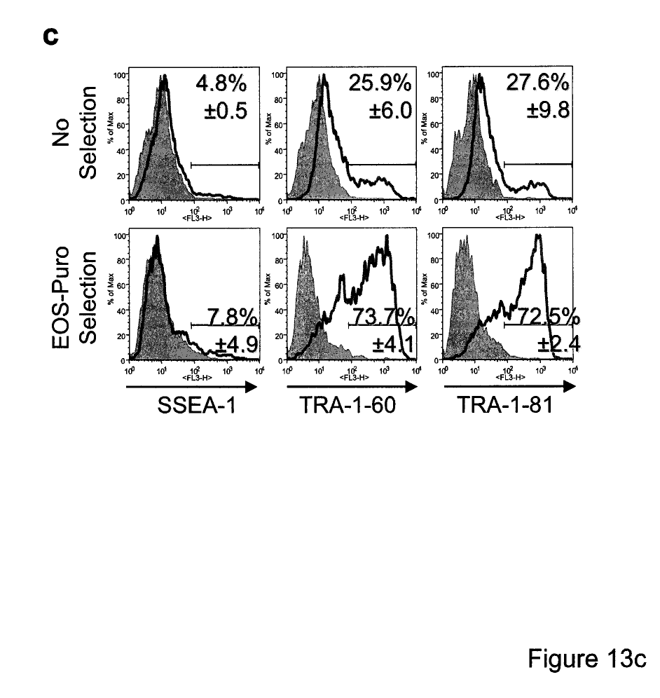

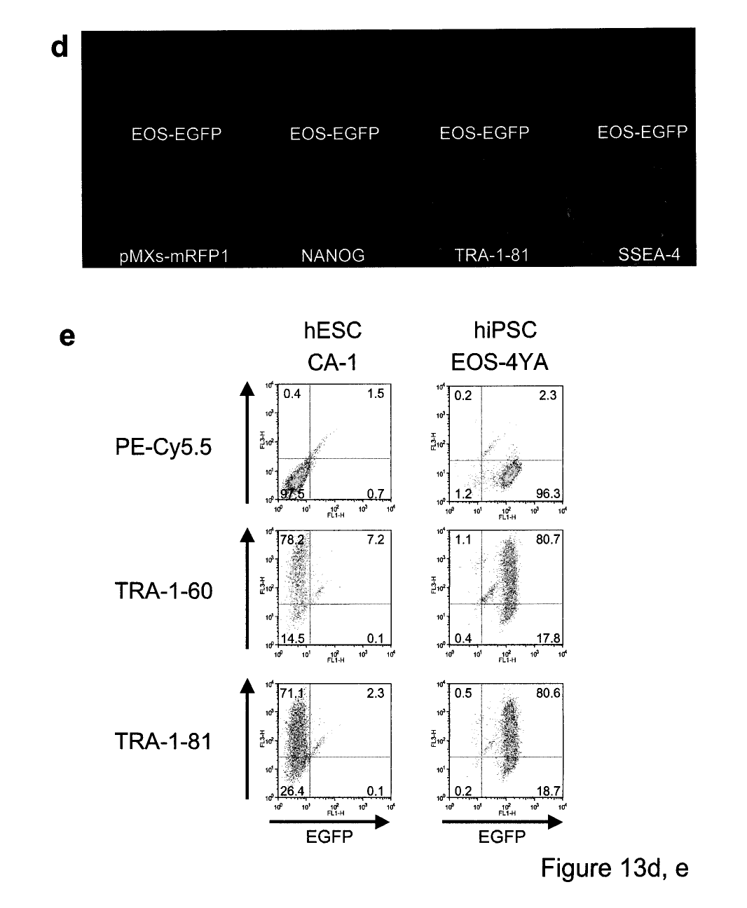

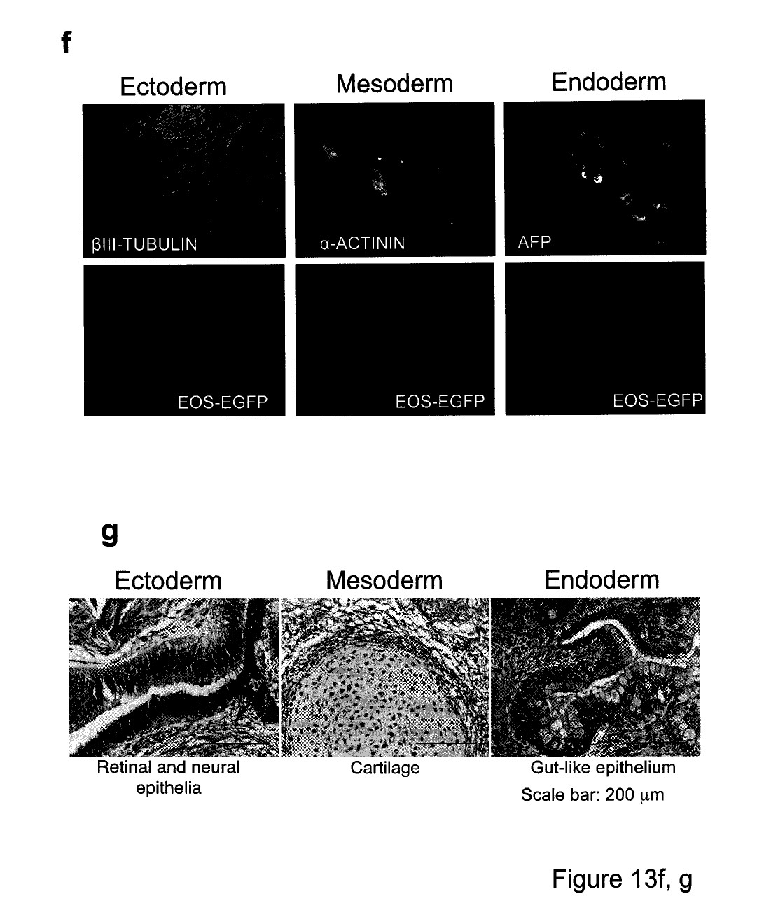

To assess the effect of EOS vector selection on reprogramming of human somatic cells, human fibroblasts expressing the mS1c7a1 (ecotropic gammaretrovirus receptor) were infected with EOS lentiviral vectors encoding EGFP-IRES-Puro, prior to infection with MoMLV-based retroviral vectors (pMXs) encoding the four human Yamanaka factors (FIG. 13a). At the same time, pMXs-mRFP1 (monomeric Red Fluorescence Protein 1) vector was infected along with the 4 factors to assess the gene transfer efficiency and to monitor retroviral vector silencing in the reprogrammed iPS cells as described. EOS-EGFP expression and putative colony formation was detected 2 weeks after induction (FIG. 13b) and puromycin selection applied at day 17. By 4 weeks of induction, colonies with non-hES like morphology continued to express pMXs-mRFP1 with occasional EOS-EGFP and SSEA-3 expressing cells, whereas those with hES like morphology coexpressed EOS-EGFP and SSEA-3 while pMXs-mRFP1 was silent. Puro selection dramatically enriched the frequency of SSEA-3 positive iPS cell colonies with good hES-like morphology from 4.8% to 46% (Table 4). Puro selection also increased the percentage of TRA-1-60 and TRA-1-81 positive human cells by 3-fold to approximately 72% after 4 weeks in 3 independent reprogramming experiments (FIG. 13c).

TABLE 4
Frequency of colonies with good morphology in absence
or presence of puromycin selection.

| | Ratio of SSEA-3 positive ES-like |
|---|---|
| Selection | colonies per total |
| Puro (−) | 4.8% (11/226) |
| Puro (+) | 46% (135/290) |

These selected iPS cell lines were isolated and continued to express EOS-EGFP coincident with the endogenous pluripotent markers NANOG, TRA-1-81, SSEA-4, and TRA-1-60 (FIG. 13e), but have silenced the pMXs-mRFG1 gammaretrovirus vector (FIG. 13d). After EB mediated differentiation, the established human iPS cells extinguished EOS-EGFP expression as expected, and spontaneously formed several different cell types corresponding to the 3 germ layers such as beta-III tubulin (ectoderm), alpha-actinin (mesoderm), and alpha-fetoprotein (endoderm) indicating functional pluripotency (FIG. 130. As the most stringent test of pluripotency for human cells, we injected the cells into NOD/SCID mice for teratoma formation. Injected mice developed mature tumors that contained a variety of typical structures and tissue types from all three germ layers—ectoderm (e.g. retinal and neural epithelia), mesoderm (e.g. cartilage) and endoderm (e.g. gut-like epithelium, ciliated epithelium) (FIG. 13g). These data demonstrate that the EOS lentiviral vector marks human iPS cells despite silencing of the MoMLV based retroviral vector (pMXs-mRFP1). Puro selection enriches for EOS infected iPS cell lines in the undifferentiated state that also co-express pluripotent stem cell markers.

### Example 14

### EOS Selection Establishes Rett Syndrome-Specific Mouse and Human iPS Cell Lines

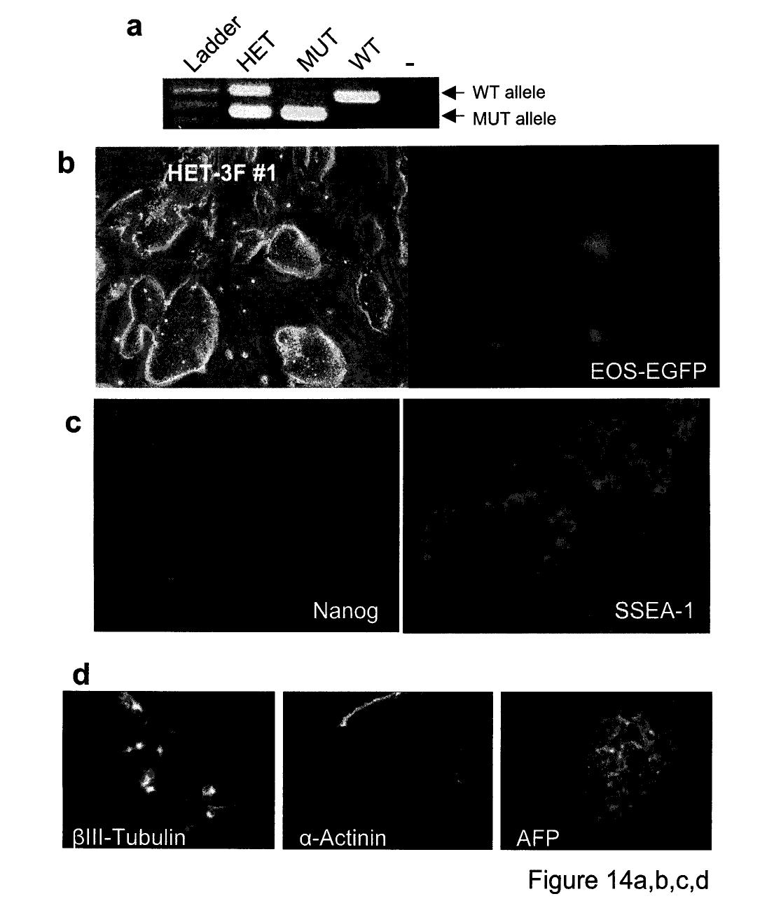

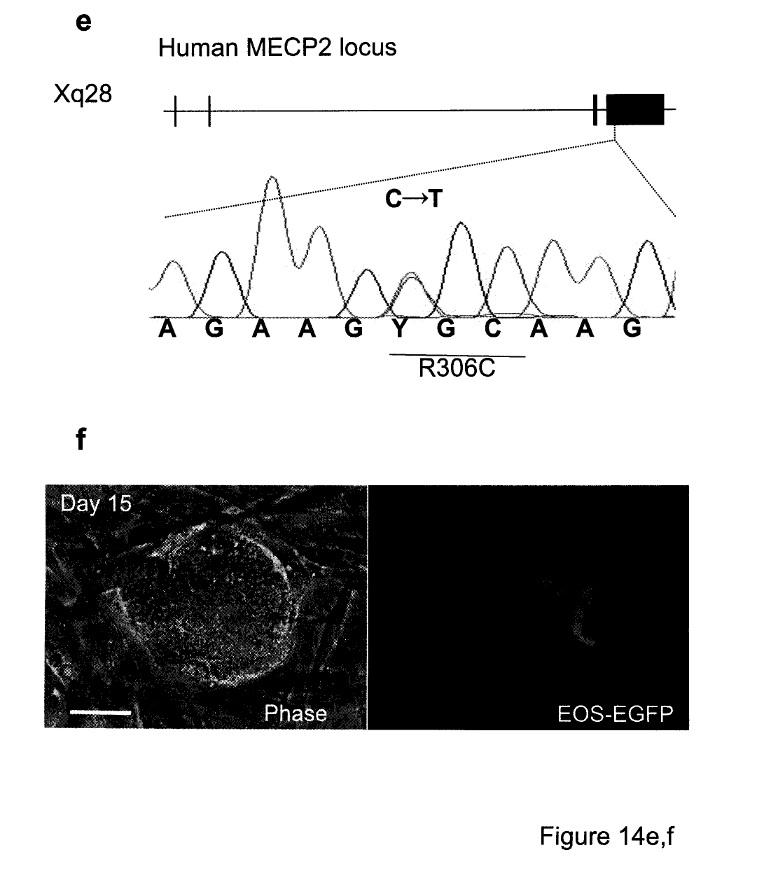

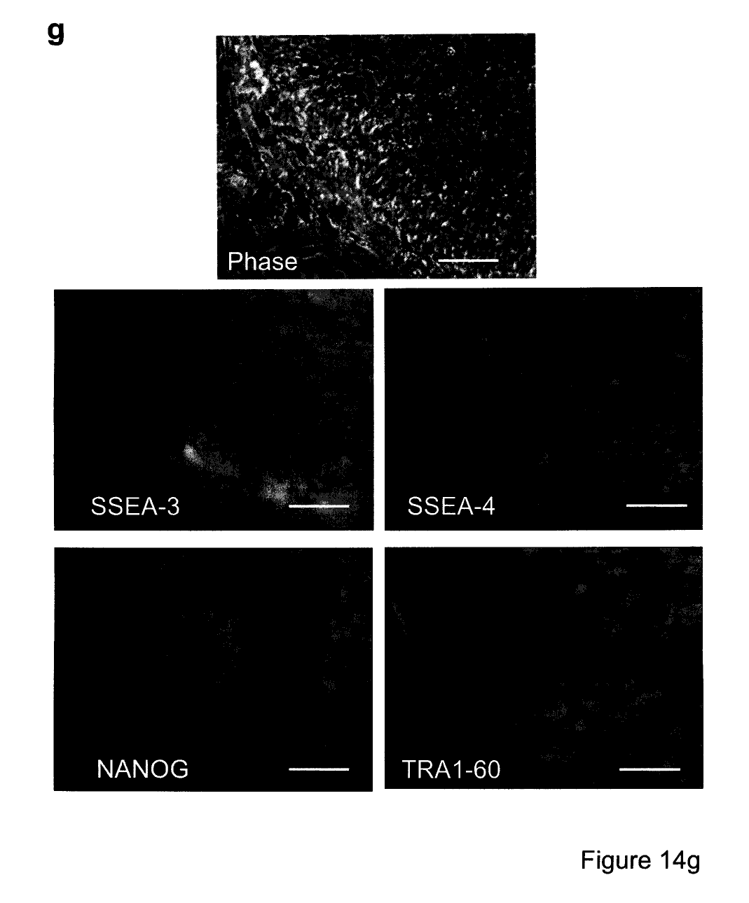

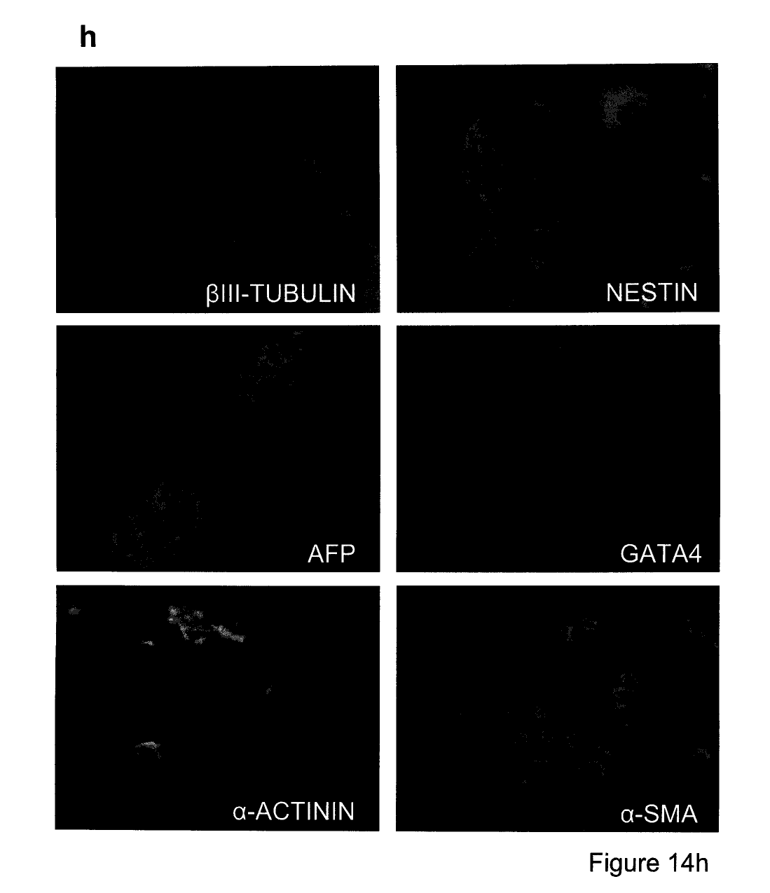

As a proof-of-principle for EOS selection reproducibility in a disease context, we generated Rett Syndrome-specific iPS cell lines. Heterozygous MEFs from Mecp2³⁰⁸ mice (Shahbazian et al., 2002) were isolated and genotyped for reprogramming experiments (FIG. 14a). The cells were reprogrammed by the 3 factor (Oct-4, Sox2, Klf4) retroviral infection, and EOS puromycin selection allowed isolation of EGFP positive colonies that were established into iPS cell lines. Phase-contrast and fluorescence microscopy of Mecp2³⁰⁸ HET iPS cell line (HET-3F #1) showed ES-like colony morphology and activated EOS-EGFP expression (FIG. 14b). EOS-EGFP-expressing HET-3F #1 mouse iPS cell line stained positive for pluripotency markers Nanog and SSEA-1 (FIG. 14c). Functional pluripotency of HET-3F #1 was revealed by positive staining for lineage-committed cell markers, betaIII-tubulin (ectoderm), alpha-actinin (mesoderm), and alpha-fetoprotein (endoderm) following EB-mediated in vitro differentiation. (FIG. 14d). Similarly, human fibroblast cells from a Rett Syndrome patient with the common C916T transition causing an R306C (arginine to cysteine) amino acid change in the transcriptional repression domain of MECP2 (FIG. 14e) were reprogrammed by the 4 Yamanaka factor retroviral infection. Fifteen days post-induction, EOS-EGFP was activated in embryonic stem cell-like colonies during reprogramming (FIG. 14f)), and the puromycin selection identified EGFP positive colonies to produce REtt Syndrome patient-derived R306C iPS cell lines that express pluripotency markers (FIG. 14g) and differentiate in vitro into the several cell types corresponding to the three germ layers including neurons (FIG. 14h). Genotype of the final mouse and human iPS cell lines was confirmed by PCR and sequencing respectively.

These results demonstrate that EOS selection can be used to establish disease-specific iPS cell lines from a subject—such as a patient, or a mouse model of a disease such as a knockout mouse.

These data further illustrate that EOS lentiviral vectors direct pluripotent stem cells specific expression and resist vector silencing while under puromycin selection.

### Example 15

### Purging Residual Undifferentiated Cells Following In Vitro Differentiation

Negative selection may also be used in combination with other selection means to identify selected cells. A nucleic acid comprising a nucleotide sequence encoding a fusion protein of hygromycin phosphotransferase and thymidine kinase (SEQ ID NO: 40, 41) may be transfected into a population of cells that is to be, or has been induced to pluripotency as described. Successful transformants are first selected by growing in hygromycin-containing medium as described. During, or after a subsequent directed differentiation procedure, gancyclovir (or a similar reagent) is added to the growth medium to select against cells expressing thymidine kinase (e.g. those that did not undergo subsequent directed differentiation), providing a doubly-selected population of differentiated cells.

### REFERENCES

Ambrosetti, D. C., Scholer, H. R., Dailey, L. & Basilico, C. Modulation of the activity of multiple transcriptional activation domains by the DNA binding domains mediates the synergistic action of Sox2 and Oct-3 on the fibroblast growth factor-4 enhancer. J Biol Chem 275, 23387-23397 (2000).
Aoi, T., Yae, K., Nakagawa, M., Ichisaka, T., Okita, K., Takahashi, K., Chiba, T., Yamanaka, S. Generation of Pluripotent Stem Cells from Adult Mouse Liver and Stomach Cells Science 321: 699-702; 2008.
Baird S D et al., 2006. Searching for IRES. RNA 12:1755-1785.
Buzina, A, Lo, M Y M, Moffet, A, Hotta, A, Fussner, E, Bharadwaj, R R, Pasceri, P, Garcia, J V, Bazett-Jones, D P, Ellis, J. β-globin LCR and intron elements cooperate and direct spatial reorganization for gene therapy PLoS Genet 4:e1000051; 2008.
Cowan et al., 2005. Science Nuclear reprogramming of somatic cells after fusion with human embryonic stem cells. Science 309, 1369-1373 (2005)
Dimos, J T et al., 2008. Induced Pluripotent Stem Cells Generated from Patients with ALS can be Differentiated into Motor Neurons Science 2008. 321:1218-21.
Hotta, A. et al. Characterization of transient expression system for retroviral vector production. J Biosci Bioeng 101, 361-368 (2006).
Kuroda, T. et al. Octamer and Sox elements are required for transcriptional cis regulation of Nanog gene expression. Mol Cell Biol 25, 2475-2485 (2005).
Lowry, W E., Richter, L., Yachechko, R., Pyle, A D., Tchieu, J., Sridharan, R., Clark, A T., Plath, K. Generation of human induced pluripotent stem cells from dermal fibroblasts Proc Natl Acad Sci USA 105:2883-8; 2008.
Maksakova, I. A. & Mager, D. L. Transcriptional regulation of early transposon elements, an active family of mouse long terminal repeat retrotransposons. J Virol 79, 13865-13874 (2005).
Meissner, A., Wernig, M. & Jaenisch, R. Direct reprogramming of genetically unmodified fibroblasts into pluripotent stem cells. Nat Biotechnol (2007) 25:1177-81.
Morita, S., Kojima, T. & Kitamura, T. Plat-E: an efficient and stable system for transient packaging of retroviruses. Gene Ther 7, 1063-1066 (2000).
Nakagawa, M. & Koyanagi, M. & Tanabe, K. & Takahashi, K. & Ichisaka, T. & Aoi, T. & Okita, K. & Mochiduki, Y. & Takizawa, N. & Yamanaka, S. Generation of induced pluripotent stem cells without Myc from mouse and human fibroblasts Nat Biotechnol 2008; 26:101-6.
Nakatake, Y. et al. Klf4 cooperates with Oct3/4 and Sox2 to activate the Lefty1 core promoter in embryonic stem cells. Mol Cell Biol 26, 7772-7782 (2006).
Nishimoto, M., Fukushima, A., Okuda, A. & Muramatsu, M. The gene for the embryonic stem cell coactivator UTF1 carries a regulatory element which selectively interacts with a complex composed of Oct-3/4 and Sox-2. Mol Cell Biol 19, 5453-5465 (1999).
Okita, K., Ichisaka, T. & Yamanaka, S. Generation of germline-competent induced pluripotent stem cells. Nature 448, 313-317 (2007).
Okumura-Nakanishi, S., Saito, M., Niwa, H. & Ishikawa, F. Oct-3/4 and Sox2 regulate Oct-3/4 gene in embryonic stem cells. J Biol Chem 280, 5307-5317 (2005).
Osborne, C. S. et al. Amelioration of retroviral vector silencing in locus control region beta-globin-transgenic mice and transduced F9 embryonic cells. J Virol 73, 5490-5496 (1999).
Park, I H., Zhao, R., West, J A., Yabuuchi, A., Huo, H., Ince, T A., Lerou, P H., Lensch, M W., Daley, G Q. Reprogramming of human somatic cells to pluripotency with defined factors Nature 2008; 451:141-6.
Palazzoli F, Carnus E, Wells D J, Bigot Y., Sustained transgene expression using non-viral enzymatic systems for stable chromosomal integration. Curr Gene Ther.; 8, 367-390 (2008).
Portsmouth D. et al., Suicide genes for cancer therapy. Mol Aspects Med. 2007; 28:4-41
Peerani, R. et al. Niche-mediated control of human embryonic stem cell self-renewal and differentiation. EMBO J 26, 4744-4755 (2007).
Rodda, D. J. et al. Transcriptional regulation of nanog by OCT4 and SOX2. J Biol Chem 280, 24731-24737 (2005).
Shahbazian, M. et al., Mice with truncated MeCP2 recapitulate many Rett syndrome features and display hyperacetylation of histone H3 Neuron 35:243-254 (2002).
Stadtfeld, M. et al., 2008. Induced Pluripotent Sem Cells Generated without Viral Integration Science 322:945-9.
Szymczak A L et al., 2004 Correction of multi-gene deficiency in vivo using a single self-cleaving 2A peptide-based retroviral vector. Nature Biotechnology 22:589-94
Takahashi, K., Okita, K., Nakagawa, M., Yamanaka, S. Induction of pluripotent stem cells from fibroblast cultures Nat Protoc 2007; 2:3081-9.
Takahashi, K., Tanabe, K., Ohnuki, M., Narita, M., Ichisaka, T., Tomoda, K., Yamanaka, S. Induction of pluripotent stem cells from adult human fibroblasts by defined factors Cell 2007; 131:861-72.
Takahashi, K. & Yamanaka, S. Induction of pluripotent stem cells from mouse embryonic and adult fibroblast cultures by defined factors. Cell 126, 663-676 (2006).
Tokuzawa, Y. et al. Fbx15 is a novel target of Oct3/4 but is dispensable for embryonic stem cell self-renewal and mouse development. Mol Cell Biol 23, 2699-2708 (2003).
Tomioka, M. et al. Identification of Sox-2 regulatory region which is under the control of Oct-3/4-Sox-2 complex. Nucleic Acids Res 30, 3202-3213 (2002).
Wang, Z. X. et al. Oct4 and Sox2 directly regulate expression of another pluripotency transcription factor, Zfp206, in embryonic stem cells. J Biol Chem 282, 12822-12830 (2007).
Wernig, M. et al. In vitro reprogramming of fibroblasts into a pluripotent ES-cell-like state. Nature 448, 318-324 (2007).
Wernig, M. et al. Neurons derived from reprogrammed fibroblasts functionally integrate into the fetal brain and improve symptoms of rats with Parkinson's disease. Proc Natl Acad Sci USA 105, 5856-5861 (2008).
Yang, X. et al., Nuclear reprogramming of cloned embryos and its implications for therapeutic cloning. Nature Genettics 39, 295-302 (2007).
Yao, S. et al. Retrovirus silencing, variegation, extinction, and memory are controlled by a dynamic interplay of multiple epigenetic modifications. Mol Ther 10, 27-36 (2004).
Yeom, Y. I. et al. Germline regulatory element of Oct-4 specific for the totipotent cycle of embryonal cells. Development 122, 881-894 (1996).

All citations are herein incorporated by reference.

One or more currently preferred embodiments have been described by way of example. It will be apparent to persons skilled in the art that a number of variations and modifications can be made without departing from the scope of the invention as defined in the claims.

What is claimed is:

1. A nucleic acid comprising an ETn poly A mutated (pAMu) promoter sequence (SEQ ID NO: 2) operatively linked to a tag sequence and an enhancer unit active in a pluripotent stem cell.

2. The nucleic acid of claim 1, wherein the enhancer unit is selected from the group consisting of one or more than one SRR2 enhancer sequences, one or more than one CR4 enhancer sequences, and a combination thereof, and the enhancer unit is located upstream of the pAMu promoter sequence.

3. The nucleic acid of claim 2 wherein the one or more than one CR4 enhancer sequence is selected from the group consisting of SEQ ID NOS: 3 and 5.

4. The nucleic acid of claim 2 wherein the one or more than one SRR2 enhancer sequence is selected from the group consisting of SEQ ID NOS: 4 and 6.

5. The nucleic acid of claim 1 wherein the tag sequence encodes an amino acid sequence of interest, the amino acid sequence of interest permitting antibiotic selection, color selection, fluorescence selection, negative selection, or cell-surface selection.

6. A cell comprising the nucleic acid of claim 1.

7. A vector comprising the nucleic acid of claim 1.

8. A nucleic acid comprising, an ETn poly A mutated (pAMu) promoter sequence (SEQ ID NO: 2) operatively linked to a tag sequence and one or more than one enhancer sequence, the enhancer sequence selected from the group comprising CR4, SRR2, and a combination of CR4 and SRR2.

9. A method of producing an induced pluripotent stem cell, comprising, either:
Ai) reprogramming a cell to induce pluripotency producing a pluripotent cell;
Aii) transfecting the pluripotent cell with the nucleic acid of claim 1 to produce a transfected pluripotent cell;
or
Bi) transfecting a cell with the nucleic acid of claim 1 to produce a transfected cell;
Bii) reprogramming a cell to induce pluripotency to produce a transfected pluripotent cell;
iii) growing the transfected pluripotent cell; and
iv) selecting for an induced pluripotent stem cell.

10. The method of claim 9 wherein the step of reprogramming comprises, either:
a. transfecting a cell with one or more than one pluripotency factors;
b. adding one or more than one chemical, cytokine, or hormone into culture medium of a cell;
c. transfecting a cell with one or more than one pluripotency factors and adding one or more than one chemical, cytokine, or hormone into culture medium of a cell;
d. nuclear transfer of a cell into a pluripotent stem cell or an oocyte; or
e. cell-cell fusion of a cell with a pluripotent stem cell.

11. A method of identifying a pluripotent stem cell comprising:
a. providing a population of pluripotent stem cells comprising the nucleic acid of claim 1; and
b. selecting for a protein encoded by the tag sequence thereby identifying the pluripotent stem cell, or selecting against a protein encoded by the tag sequence thereby removing or killing the pluripotent stem cell.

12. The method of claim 11, wherein the pluripotent stem cell is an induced pluripotent stem cell or an embryonic stem cell.

13. A stem cell expression cassette, comprising an ETn poly A mutated (pAMu) promoter sequence (SEQ ID NO: 2) operatively linked to a tag sequence and one or more than one enhancer sequences, the enhancer sequence selected from the group comprising CR4, SRR2, and a combination of CR4 and SRR2.

14. A kit for identifying a pluripotent stem cell or an embryonic stem cell, comprising a nucleic acid according to claim 1 and instructions for its use.


SEQUENCE LISTING (42 sequences)

SEQ ID NO: 1 - DNA, 318 bp, Artificial
  WT ETn type II LTR promoter region (ETn)
tgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaacaataaaatttgagccttgatcagagtaactgtcttggctacattctttcttccgccccgtctagattcctctcttacagctcgagcggccttctcagtcgaaccgttcacgttgcgagctgctggcggccgcaaca

SEQ ID NO: 2 - DNA, 318 bp, Artificial
  Poly A mutated ETn type II LTR promoter region (ETn pAMu)
tgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaaca

SEQ ID NO: 3 - DNA, 147 bp, Artificial
  CR4 - Conserved region 4 in positive orientation
gggtgtggggaggttgtagcccgaccctgcccctccccccagggaggttgagagttctgggcagacggcagatgcataacaaaggtgcatgatagctctgccctgggggcagagaagatggttggggaggggtccctctcgtcctag

SEQ ID NO: 4 - DNA, 129 bp, Artificial
  SRR2 - Sox Regulatory Region 2 in positive orientation
ccagtccaagctaggcaggttcccctctaattaatgcagagactctaaaagaatttcccgggctcgggcagccattgtgatgcatataggattattcacgtggtaatgagcacagtcgacagttcttgc

SEQ ID NO: 5 - DNA, 147 bp, Artificial
  CR4 in negative orientation
ctaggacgagagggacccctccccaaccatcttctctgcccccagggcagagctatcatgcacctttgttatgcatctgccgtctgcccagaactctcaacctccctggggggaggggcagggtcgggctacaacctccccacaccc

SEQ ID NO: 6 - DNA, 129 bp, Artificial
  SRR2 in negative orientation
gcaagaactgtcgactgtgctcattaccacgtgaataatcctatatgcatcacaatggctgcccgagcccgggaaattcttttagagtctctgcattaattagaggggaacctgcctagcttggactgg

SEQ ID NO: 7 - DNA, 506 bp, Artificial
  A regulatory sequence of HSC1-CR4(+)-pAMu-EGFP
gaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 8 - DNA, 507 bp, Artificial
  A regulatory sequence of HSC1-C(-)-pAMu-EGFP
ggaattcactagtgattaagctcgagctaggacgagagggacccctccccaaccatcttctctgcccccagggcagagctatcatgcacctttgttatgcatctgccgtctgcccagaactctcaacctccctggggggaggggcagggtcgggctacaacctccccacacccgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 9 - DNA, 850 bp, Artificial
  A regulatory sequence of PL-EOS-C(3+)A-EiP
gaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 10 - DNA, 852 bp, Artificial
  A regulatory sequence of HSC1-C(3-)-pAMu-EGFP
ggaattcactagtgattaagctcgagctaggacgagagggacccctccccaaccatcttctctgcccccagggcagagctatcatgcacctttgttatgcatctgccgtctgcccagaactctcaacctccctggggggaggggcagggtcgggctacaacctccccacacccggaattcactagtgattaagctcgagctaggacgagagggacccctccccaaccatcttctctgcccccagggcagagctatcatgcacctttgttatgcatctgccgtctgcccagaactctcaacctccctggggggaggggcagggtcgggctacaacctccccacacccgaattcactagtgattaagctcgagctaggacgagagggacccctccccaaccatcttctctgcccccagggcagagctatcatgcacctttgttatgcatctgccgtctgcccagaactctcaacctccctggggggaggggcagggtcgggctacaacctccccacacccgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 11 - DNA, 484 bp, Artificial
  A regulatory sequence of HSC1-S(+)-pAMu-EGFP
ggaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 12 - DNA, 484 bp, Artificial
  A regulatory sequence of HSC1-S(-)-pAMu-EGFP
ggaattcgattctactcgagagcaagaactgtcgactgtgctcattaccacgtgaataatcctatatgcatcacaatggctgcccgagcccgggaaattcttttagagtctctgcattaattagaggggaacctgcctagcttggactgggaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 13 - DNA, 632 bp, Artificial
  A regulatory sequence of HSC1-SRR2(2+)-pAMu-EGFP
gaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 14 - DNA, 931 bp, Artificial
  A regulatory sequence of PL-EOS-S(4+)A-EiP
ggaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatct

SEQ ID NO: 15 - DNA, 1296 bp, Artificial
  A regulatory sequence of HSC1-pAMu-EGFP-CR4(+) (pAMu-EGFP-CR4(+)
ggaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagctta

SEQ ID NO: 16 - DNA, 1313 bp, Artificial
  A regulatory sequence of HSC1-pAMu-EGFP-CR4(-) (pAMu-EGFP-CR4(-)
ggaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcactagtgattaagctcgagctaggacgagagggacccctccccaaccatcttctctgcccccagggcagagctatcatgcacctttgttatgcatctgccgtctgcccagaactctcaacctccctggggggaggggcagggtcgggctacaacctccccacacccgaattcct

SEQ ID NO: 17 - DNA, 1279 bp, Artificial
  A regulatory sequence of HSC1-pAMu-EGFP-SRR2(+) (pAMu-EGFP-SRR2(+)
ggaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcccagtccaagctaggcaggttcccctctaattaatgcagagactctaaaagaatttcccgggctcgggcagccattgtgatgcatataggattattcacgtggtaatgagcacagtcgacagttcttgctctcgagtaga

SEQ ID NO: 18 - DNA, 1290 bp, Artificial
  A regulatory sequence of HSC1-pAMu-EGFP-SRR2(-) (pAMu-EGFP-SRR2(-)
ggaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcgattctactcgagagcaagaactgtcgactgtgctcattaccacgtgaataatcctatatgcatcacaatggctgcccgagcccgggaaattcttttagagtctctgcattaattagaggggaacctgcctagcttggactgggaattcct

SEQ ID NO: 19 - DNA, 1805 bp, Artificial
  A regulatory sequence of HSC1-C(3+)-pAMu-EGFP-S(-) (C(3+)-pAMu-EGFP-S(-))
gaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcgattctactcgagagcaagaactgtcgactgtgctcattaccacgtgaataatcctatatgcatcacaatggctgcccgagcccgggaaattcttttagagtctctgcattaattagaggggaacctgcctagcttggactgggaattcct

SEQ ID NO: 20 - DNA, 1947 bp, Artificial
  A regulatory sequence of HSC1-C(3+)-pAMu-EGFP-S(2+) (C(3+)-pAMu-EGFP-S(2+))
gaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagcttaatcactagtgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattaattcccagtccaagctaggcaggttcccctctaattaatgcagagactctaaaagaatttcccgggctcgggcagccattgtgatgcatataggattattcacgtggtaatgagcacagtcgacagttcttgctctcgagtaga

SEQ ID NO: 21 - DNA, 1594 bp, Artificial
  A regulatory sequence of HSC1-S(2+)-pAMu-EGFP-C(+) HSC1-S(2+)-pAMu-EGFP-C(+))
ggaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagctcgagctta

SEQ ID NO: 22 - DNA, 1786 bp, Artificial
  A regulatory sequence of HSC1-S(2+)-pAMu-EGFP-C(2-) (S(2+)-pAMu-EGFP-C(2-))
gaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcccagtccaagctaggcaggttcccctctaattaatgcagagactctaaaagaatttcccgggctcgggcagccattgtgatgcatataggattattcacgtggtaatgagcacagtcgacagttcttgctctcgagtagaatcgaattcgttctgtagtctcccctcccccagcctgaaacctgcttgctcaggggtggagcttcctgctcattcgttctgccacgcccactgctggaacctgcggagccacacccgtgcacctttctactggaccagagattattcggcgggaatcgggtcccctcccccttccttcataactagtgtcgcaactataaaatttgagccttgatcagagtaactgtcttggctacattctttcttccgccccgtctagattcctctcttacagctcgagcggccttctcagtcgaaccgttcacgttgcgagctgctggcggccgcaacaagatctgcgatctaagtaagcttggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgaaggaattcactagtgattaagctcgagctaggacgagagggacccctccccaaccatcttctctgcccccagggcagagctatcatgcacctttgttatgcatctgccgtctgcccagaactctcaacctccctggggggaggggcagggtcgggctacaacctccccacacccgaattaattcactagtgattaagctcgagctaggacgagagggacccctccccaaccatcttctctgcccccagggcagagctatcatgcacctttgttatgcatctgccgtctgcccagaactctcaacctccctggggggaggggcagggtcgggctacaacctccccacacccgaattcct

SEQ ID NO: 23 - DNA, 1946 bp, Artificial
  Tag sequence encoding EGFP operatively linked to an IRES element and a sequence encoding puromycin resistance (“PuroR”).
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgatcgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 24 - DNA, 2145 bp, Artificial
  a tag sequence encoding a neomycin resistance gene product (“NeoR”) operatively linked to an IRES element and a sequence encoding EGFP
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 25 - DNA, 26 bp, Artificial
  ETn-pA-Mu-s primer
tagtgtcgcaactataaaatttgagc

SEQ ID NO: 26 - DNA, 26 bp, Artificial
  ETn-pA-Mu-a primer
gctcaaattttatagttgcgacacta

SEQ ID NO: 27 - DNA, 19 bp, Artificial
  RVP3(Promega) primer
ctagcaaataggctgtccc

SEQ ID NO: 28 - DNA, 22 bp, Artificial
  GLP2(Promega) primer
ctttatgtttttggcgtcttcc

SEQ ID NO: 29 - DNA, 28 bp, Artificial
  Nanog-Nco1 primer
gcccatggtgttagtatagaggaagagg

SEQ ID NO: 30 - DNA, 26 bp, Artificial
  Nanog-BamH1 primer
taggatccaaaagtcagcttgtgtgg

SEQ ID NO: 31 - DNA, 27 bp, Artificial
  mOct4-CR4-s(EcoRI) primer
ggagaattcgggtgtggggaggttgta

SEQ ID NO: 32 - DNA, 29 bp, Artificial
  mOct4-CR4-a(XhoI) primer
aagctcgagctaggacgagagggacccct

SEQ ID NO: 33 - DNA, 29 bp, Artificial
  mSox2-SRR2-s(EcoRI) primer
attgaattcccagtccaagctaggcaggt

SEQ ID NO: 34 - DNA, 31 bp, Artificial
  mSox2-SRR2-a(XhoI) primer
ctactcgagagcaagaactgtcgactgtgct

SEQ ID NO: 35 - DNA, 18 bp, Artificial
  IMR3912 (common forward primer)
aacggggtagaaagcctg

SEQ ID NO: 36 - DNA, 18 bp, Artificial
  IMR3913 (WT allele specific reverse primer)
tgatggggtcctcagagc

SEQ ID NO: 37 - DNA, 18 bp, Artificial
  IMR3914 (MUT allele specific reverse primer)
atgctccagactgccttg

SEQ ID NO: 38 - DNA, 20 bp, Artificial
  RTT-Fwd primer
cgctctgccctatctctgac

SEQ ID NO: 39 - DNA, 20 bp, Artificial
  RTT-Rev primer
agtcctttcccgctcttctc

SEQ ID NO: 40 - DNA, 2199 bp, Artificial
  HygroTK nucleic acid
atgctttacatgtgtttagtcgaggttaaaaaaacgtctaggccccccgaaccacggggacgtggttttcctttgaaaaacacgatgataagcttgccacaacccgtaccaaagatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactga

SEQ ID NO: 41 - protein, 730 aa, Artificial
  HygroTK amino acid
MLYMCLVEVKKTSRPPEPRGRGFPLKNTMISLPQVPKMDRSGKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVASAFDQAARSRGHSNRRTALRPRRQQEATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 42 - DNA, 3443 bp, Artificial
  EGFP-IRES-HygroTK construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacctcgacgttattcccttcgatcgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggaatctgatctggggcctcggtgcacatgctttacatgtgtttagtcgaggttaaaaaaacgtctaggccccccgaaccacggggacgtggttttcctttgaaaaacacgatgataagcttgccacaacccgtaccaaagatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactga